(19) United States
(12) Patent Application Publication
(10) Pub. No.: US 2011/0245235 A1
(43) Pub. Date: Oct. 6, 2011

(54) Systems, Methods, and Devices for Rehabilitation of Auditory System Disorders Using Pharmaceutical Agents and Auditory Devices

(76) Inventors: Peter J. Hanley, New South Wales (AU); Robert H. Frater, New South Wales (AU); Arjun Goyal, New South Wales (AU); Paul B. Davis, Fort Lauderdale, FL (US)

(21) Appl. No.: 13/123,111

(22) PCT Filed: Oct. 9, 2009
(86) PCT No.: PCT/AU09/01337

§ 371 Date: Jun. 22, 2011

(30) Foreign Application Priority Data
Oct. 10, 2008 (US) 61/136874

(51) Int. Cl. A61K 31/55; A61K 31/5517; A61P 27/16; A61K 31/135; A61K 31/5513; A61K 31/197; A61K 31/137; A61K 31/4402; A61K 31/277; A61K 31/53; A61M 21/02

(52) U.S. Cl. 514/217; 514/220; 514/657; 514/221; 514/561; 514/654; 514/357; 514/523; 514/242; 600/28

(57) ABSTRACT

The present inventions provide combination therapies, kits, and methods for providing relief and/or for improving one or more symptoms associated with an auditory system disorder in a subject diagnosed with the auditory system disorder, comprising an effective amount of at least one pharmaceutical agent and at least one auditory system disorder device, as well as methods for using at least one pharmaceutical agent and/or at least one auditory system disorder device in the manufacture of said kits.

### CROSS-REFERENCE TO RELATED APPLICATIONS

This application claims priority to U.S. Provisional Application No. 61/136,874, filed on Oct. 10, 2008. This Application is also related to U.S. Provisional Application No. 61/071,254, filed on Apr. 18, 2008; and U.S. application Ser. No. 11/921,500, filed on Dec. 4, 2007, which is a U.S. National Stage Application of International Application No. PCT/AU2006/000777, filed on Jun. 7, 2006, which claims the benefit of U.S. Provisional Application No. 60/689,088; and U.S. application Ser. No. 10/727,036, filed on Dec. 4, 2003, which is a continuation-in-part of U.S. application Ser. No. 09/936,687, now U.S. Pat. No. 6,682,472, filed on Sep. 17, 2001 which is a U.S. National Stage application of International Application No. PCT/AU00/00207, filed on Mar. 17, 2000, which claims the benefit of Australian Provisional Application No. PP9275, filed on Mar. 17, 1999. Each of these applications, in their entirety, is herein incorporated by reference.

### BACKGROUND

1. Field

The present disclosure relates to certain pharmaceutical agents, systems, methods, devices, and/or apparatuses for rehabilitation of auditory system disorders. The present disclosure also relates to systems, methods, devices, and apparatuses for auditory system rehabilitation by providing a stimulus to the auditory system of an individual experiencing tinnitus or conditions of reduced tolerance of loud sounds and combining that rehabilitation treatment with certain pharmaceutical agents.

2. Description of Related Art

A large percentage of the population experiences some form of an auditory system disorder. For many people, the disorder can be extremely disturbing and can, in some instances, lead to additional disorders. Tinnitus and conditions of reduced sound tolerance, including hyperacusis, are examples of often disturbing types of auditory system disorders. Hyperacusis involves a severe intolerance of moderately loud external noises. Tinnitus is commonly associated with hyperacusis, and people with tinnitus perceive sounds that are not present in the external environment and/or that other people cannot generally hear. These sounds can include, for example, ringing in the ears, beating, pounding, buzzing and humming background sounds, roaring, or whistling noises in the ears.

Tinnitus can have a negative impact on work, family and social life and can lead to an inability to relax and disturbance of concentration and sleep patterns. It is estimated that a large proportion of the population (around 15%) experience some degree of tinnitus. For a small proportion, estimated around 1-2% of the general population, a secondary reaction involving the auditory cortex, the brain stem, the limbic and autonomic systems leads to significant distress and disturbance. This reaction appears to involve neuroplastic change as recently indicated with MRI and PET brain scanning.

There are few effective treatment options available for tinnitus sufferers, with the majority often being advised that “you'll have to learn to live with it”. Most patients find that they can far more readily ignore an external sound than their tinnitus.

The present inventors have observed that it would be desirable to provide more effective rehabilitation techniques for people suffering from auditory system disorders based, in part, on a better understanding of the neural processes underlying the auditory system disorders. The inventors believe that certain combinations of devices and pharmaceutical agents may provide more effective treatments for people suffering from auditory system disorders, including individuals experiencing tinnitus or conditions of reduced tolerance of loud sounds.

### SUMMARY

A portion of the people who suffer from auditory system disorders, such as tinnitus, can be highly disturbed by them. Continuous perception of auditory system disorders such as tinnitus can lead to insomnia, an inability to relax, anxiety, depression, and even suicide in extreme cases. Hyperacusis can generally occur in association with tinnitus, and is thought to share the same underlying causes. Thus, references to tinnitus in this document should be construed as including in many circumstances the phenomena of hyperacusis or other types of reduced tolerance of loud sounds.

Exemplary embodiments disclosed herein may include a kit for improving one or more symptoms associated with an auditory system disorder in a subject diagnosed with the auditory system disorder, comprising: (a) an effective amount of at least one pharmaceutical agent; and (b) at least one auditory system disorder device.

In exemplary embodiments, the pharmaceutical agent may comprise one or more anxiolytic agents, antidepressant agents, anticonvulsants, antiarrthymic agents, antihistamines, muscarinic agents, H₁ antagonists, opioid agents, osmotic regulators, vasodilators, anesthetic agents, N-methyl-D-aspartic acid (NMDA) receptor antagonists, diuretics, or combinations or mixtures thereof. In exemplary embodiments, the pharmaceutical agent may be alprazolam.

In certain embodiments, the at least one auditory system disorder device may be a masking device, a hearing aid, a cochlear implant, a tinnitus retraining therapy device, or combinations thereof. In exemplary embodiments, the at least one auditory system disorder device may be an audio playback device that comprises a receiver configured to receive an audio signal, a processor for spectrally modifying the audio signal in a substantially real time manner to compensate for an auditory system disorder, and an output for outputting the spectrally modified audio signal to the subject.

In exemplary embodiments, the at least one auditory system disorder device may be configured to provide a treatment signal and to modify a treatment signal to include troughs and peaks wherein the modified treatment signal intermittently masks the auditory system disorder of the subject during at least one of the peaks and does not fully mask the auditory system disorder during at least some of the troughs.

In exemplary embodiments, the kit may provide at least about 2%, 5%, 7%, 10%, or 15% greater relief of one or more symptoms of the auditory system disorder, relative to symptom relief in patients having the same auditory system disorder and receiving separate administrations of a pharmaceutical agent and auditory system device in a non-combinative therapy regimen.

In exemplary embodiments, the effective amount of the at least one pharmaceutical agent in the kit may be at least a 5%, 10%, 20%, 25%, 35%, 50%, or 75% lower daily dosage of the pharmaceutical agent, as compared to the daily dosage of the pharmaceutical agent required to achieve comparable symptom relief in a subject having the same auditory system disorder and being administered the at least one pharmaceutical agent alone.

In exemplary embodiments, the kit may allow for the achievement of at least the same relief of one or more symptoms of the auditory system disorder within the subject with at least 5%, 10%, 25%, or 50% less daily administering time of the auditory system disorder device, as compared to the daily administering time required to achieve the symptom relief for a subject having the same auditory system disorder and using the auditory system disorder device alone.

In exemplary embodiments, the kit may allow for the achievement of at least the same relief of one or more symptoms of the auditory system disorder within the subject with at least 5%, 10%, 25%, 35%, or 50% less administering time of the auditory system disorder device, as compared to the administering time required to achieve the symptom relief for a subject having the same auditory system disorder and using the auditory system disorder device alone.

In exemplary embodiments, use of the kit by the subject may achieve the improvements in one or more symptoms with at least 3%, 5%, 7%, 10%, 15%, or 25% less incidence of side effects associated with the at least one pharmaceutical agent, as compared to the normal incidence of side effects within subjects being administered an effective amount of the pharmaceutical agent alone.

In exemplary embodiments, the kit may achieve at least about 5%, 8%, 12%, 16%, 20%, 30%, or 40% greater compliance with proper administering procedures in subjects having at least one auditory system disorder, relative to compliance in subjects having the auditory system disorder and receiving separate administrations of a pharmaceutical agent and auditory system device in a non-combinative therapy regimen.

Exemplary embodiments may provide a combination therapy for improving one or more symptoms associated with an auditory system disorder in a subject diagnosed with the auditory system disorder, comprising (a) an effective amount of at least one pharmaceutical agent and (b) at least one auditory system disorder device.

Exemplary embodiments may provide a method for improving one or more symptoms associated with an auditory system disorder in a subject diagnosed with the auditory system disorder comprising administering to the subject: (a) an effective amount of at least one pharmaceutical agent; and (b) at least one auditory system disorder device.

In exemplary embodiments, the at least one auditory system disorder device may be administered for an effective duration. In exemplary embodiments, at least one pharmaceutical agent and at least one auditory system disorder device may be administered simultaneously. In exemplary embodiments, at least one pharmaceutical agent and at least one auditory system disorder device may be administered sequentially.

Exemplary embodiments may provide a method for lessening perceived loudness of at least one tone associated with an auditory system disorder in a subject diagnosed with the auditory system disorder comprising administering to the subject (a) an effective amount of at least one pharmaceutical agent and (b) at least one auditory system disorder device.

Exemplary embodiments may provide a method for improving one or more symptoms associated with an auditory system disorder in a subject diagnosed with the auditory system disorder, comprising administering to the subject (a) an effective amount of at least one pharmaceutical agent selected from anxiolytic agents, antidepressant agents, and mixtures thereof, and (b) an audio playback device comprising a receiver configured to receive an audio signal, a processor for spectrally modifying the audio signal in a substantially real time manner to compensate for an auditory system disorder, and an output for outputting the spectrally modified audio signal to a person.

Exemplary embodiments may provide a method for using at least one pharmaceutical agent in the manufacture of a kit for use in treating an auditory system disorder, wherein the kit comprises the at least one pharmaceutical agent and at least one auditory system disorder device.

### BRIEF DESCRIPTION OF THE DRAWINGS

In order to facilitate a more detailed understanding of the nature of the inventions disclosed herein, exemplary embodiments of systems, methods and apparatuses for rehabilitation of auditory system disorders will now be described in detail, by way of example only, with reference to the accompanying drawings in which:

FIG. 1 is a graphical representation of the long-term spectra of both a music recording and a typical prior art tinnitus masker;

FIG. 2 is a diagram illustrating the contour changes associated with auditory system disorders in accordance with certain exemplary embodiments disclosed herein;

FIG. 3 is a graphical representation of an exemplary patient's hearing thresholds and their required equalization curve calculated in accordance with certain exemplary embodiments disclosed herein;

FIG. 4 is a schematic diagram graphically illustrating intermittent tinnitus relief with music in accordance with certain exemplary embodiments disclosed herein;

FIG. 5 is a graphical representation of an exemplary patient's hearing thresholds and their required equalization curves calculated in accordance with certain exemplary embodiments disclosed herein;

FIG. 6 is an exemplary illustration of tinnitus pathogenesis and mechanism of action in accordance with certain exemplary embodiments disclosed herein;

FIG. 7 is a schematic diagram of an exemplary tinnitus rehabilitation method in accordance with certain exemplary embodiments disclosed herein;

FIG. 8 is a schematic block diagram of a possible embodiment of an auditory system disorder rehabilitation device in accordance with certain exemplary embodiments disclosed herein;

FIGS. 9A-E and 10A-D are flowcharts illustrating an exemplary method of providing an auditory system disorder rehabilitation sound recording in accordance with certain exemplary embodiments disclosed herein;

FIG. 11 is a schematic diagram of an embodiment of an auditory system disorder rehabilitation device in accordance with certain exemplary embodiments disclosed herein;

FIG. 12 is a schematic diagram of an exemplary tinnitus rehabilitation method in accordance with certain exemplary embodiments disclosed herein;

FIG. 13 is a schematic diagram of an exemplary tinnitus rehabilitation method in accordance with certain exemplary embodiments disclosed herein;

FIG. 14 is a functional schematic of a digital playback device in accordance with certain exemplary embodiments disclosed herein;

FIG. 15 a schematic diagram of an exemplary tinnitus rehabilitation system in accordance with certain exemplary embodiments disclosed herein; and

FIG. 16 is a functional schematic of a digital audio distribution system in accordance with certain exemplary embodiments disclosed herein.

### DETAILED DESCRIPTION

Terms are used herein as generally used in the art, unless otherwise defined in the following:

“Pharmaceutical agent” is used herein to refer to any agent, compound, or drug having a therapeutic effect on a subject when administered to the subject in an effective amount.

“Pharmaceutical composition” is used herein to refer to a composition comprising an effective amount of at least one pharmaceutical agent and at least one pharmaceutically acceptable excipient and/or carrier.

“Effective amount” or “pharmaceutically effective amount” of an agent or compound as provided herein refers to a sufficient amount of the agent or compound to provide the desired therapeutic effect. The exact amount required will vary from subject to subject, depending on age, general condition of the subject, the severity of the condition being treated, and the particular agent or compound administered, and the like. An appropriate “effective amount” in any individual case may be determined by one of ordinary skill in the art and/or using routine experimentation.

“Pharmaceutically acceptable” refers to those compounds, agents, materials, compositions, excipients, and/or dosage forms that are, within the scope of sound medical judgment, suitable for contact with the tissues of human beings and animals without excessive toxicity, irritation, allergic response, or other problems or complications commensurate with a reasonable benefit/risk ratio.

“Improving one or more symptoms” includes, but is not limited to, the prevention, treatment, reversal, part-reversal, reduction, diminution, and/or amelioration of a symptom.

“Subject” is an animal including the human species that is treatable with the compositions, methods and kits disclosed.

“Effective duration” is used herein to refer to a sufficient amount of time to provide the desired therapeutic effect. An appropriate “effective duration” in any individual case may be determined by one of ordinary skill in the art and/or using routine experimentation.

It will be apparent to one skilled in the art, in view of the following detailed description and the claims appended hereto, that various substitutions and/or modifications may be made to the present inventions without departing from the scope of the inventions as claimed.

Auditory system disorder devices may include, for example, masking devices, hearing aids, cochlear implants, sound generators, music players, tinnitus retraining devices, or combinations thereof.

For example, exemplary auditory system disorder devices that can be used in certain embodiments may include, for example, hearing aid-style devices that produce a band of noise in an attempt to partially or totally “mask” or cover up the perception of the tinnitus. Such masking can give a sense of relief and control over the tinnitus in a portion of patients for whom the devices allow them to cover up their tinnitus perception. Other reported devices attempt to achieve masking using devices that deliver vibrations (U.S. Pat. No. 5,692,056), pulsed ultrasonic stimulation (U.S. Pat. No. 6,394,969), or radio frequency waves (e.g., “Theraband™”) to the patient, while other reported devices (e.g., U.S. Pat. No. 5,697,975) seek to achieve stimulation through direct electrical discharge to the brain or provide relief by delivering an acoustic stimulus at a level that is inaudible to the patient (WO 01/70110).

Over the past decade, a further understanding of the neurophysiological processes underlying tinnitus has been published, emphasizing the role of the neural pathways in the emergence of distressing tinnitus and the possibility of taking advantage of neural plasticity to retrain its perception. One such retraining approach has been dubbed “Tinnitus Retraining Therapy” or TRT. In this technique, patients are often given intensive counseling, and sometimes use noise generators at a volume that does not completely mask the tinnitus.

Another auditory system disorder device is the “Silentia Set” developed by Starkey Corp., which is a pair of hearing aid devices which wirelessly receive signals from a stereo system via an induction loop under a pillow at bedtime. Recording of high frequency noise bands (“water sounds”), babble noise, traffic sounds and music have been used to mask tinnitus using this system.

Additional exemplary embodiments of auditory system disorder devices and related methods for rehabilitation of auditory system disorders by providing a stimulus to the auditory system of an individual experiencing an auditory disorder are described herein.

Tinnitus “masking” may be broadly defined as the obscuring, and/or partial obscuring, of tinnitus perception with an external sound. One accepted audiometric measure of the effectiveness of tinnitus maskers is the intensity of sound required to just mask an individual's tinnitus. This measure is known as the Minimum Masking Level (“MML”). One criterion for successful masking is that the acceptability of a masking stimulus be inversely proportional to its MML, and that the stimulus be a sufficiently pleasant substitute for the tinnitus. During clinical practice, the inventors have observed that several tinnitus sufferers have reported attempting to use music to find relief from their tinnitus, but often found that the volume required to mask their tinnitus was unacceptably high. It was also observed that most of these persons tended to have a steeply sloping hearing loss characteristic, and a tinnitus pitch which closely corresponded with the edge of the maximal hearing loss frequencies.

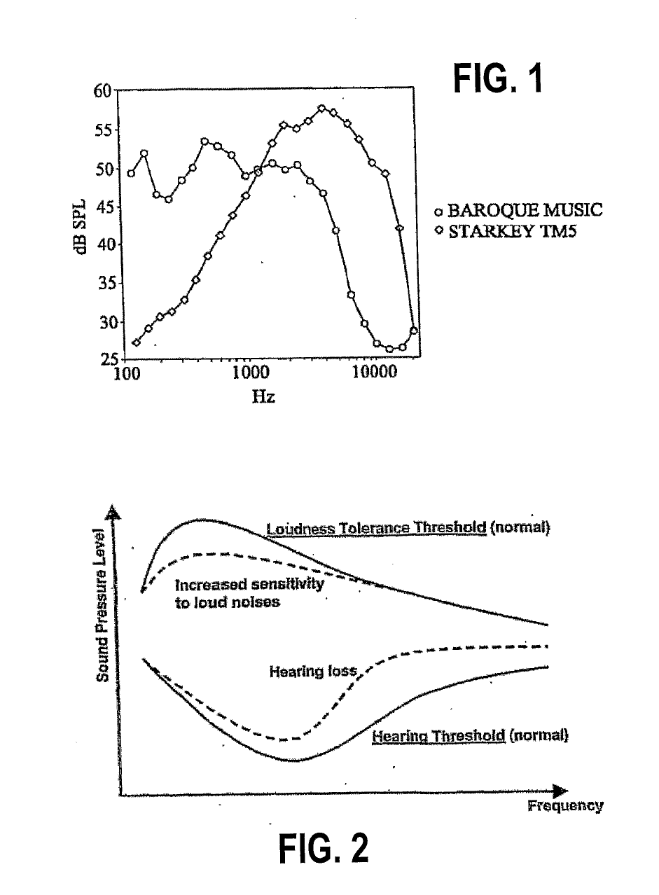

Typically, the presence of a sloping high frequency hearing loss would mean that at a relaxing sound volume level, only the low pitch components of the music are heard, and therefore the perception of full musicality and high frequency energy available for masking is inhibited. The long term spectra of both a music recording and a typical prior art tinnitus masker (e.g., a Starkey TM5) are illustrated in FIG. 1. In FIG. 1, a sound level analyzer was used to average the response of each of the two recordings over a 64 second period. The spectra were then matched at 1 kHz to enable a comparison of the frequency composition of the two spectra, irrespective of overall sound pressure levels. As can be seen from FIG. 1, if the masker is assumed to be the optimal frequency response for hearing impaired listeners, then the unfiltered music has insufficient high frequency energy and excessive low frequency response. After over a decade of research into the effect of various acoustic stimuli on auditory system disorders, an understanding of the brain changes which contribute to auditory system disorders (including but not limited to, for example, tinnitus, hyperacusis, and hearing loss) and recognition that the auditory system disorder may be different for everyone, it has been determined that an important aspect of providing tinnitus relief is providing stimulation to auditory pathways deprived of stimulation as a result of hearing loss. For example, as illustrated in FIG. 2, an individual suffering from hearing loss may have an above normal hearing threshold. In addition, the individual may also have an increased sensitivity to loud noises (e.g., hyperacusis, etc) which results in a lower than normal threshold for louder sounds at certain frequencies. The upper threshold (the loudness tolerance threshold), as seen in FIG. 2, may be most prominent at frequencies that are different from the frequencies where hearing loss occurs. Accordingly, exemplary embodiments disclosed herein are for a tinnitus protocol which modifies the frequency response characteristics of an audio signal with a view to overcoming some of the shortcomings of traditional tinnitus maskers. In certain embodiments, these shortcomings may include, for example, a lack of higher frequency signals, sounds that are not pleasant to listen to, and sounds that need to be listened to at an uncomfortably loud level, insufficient energy at frequencies where the patient has hearing loss, or excessive energy at frequencies where the patient has reduced tolerance of loud sounds.

Although the following description will be made primarily with reference to modifying the frequency response characteristics of music, it is to be understood that a tinnitus protocol in accordance with the disclosed inventions may also be applied to other types of audio signals suitable for relieving tinnitus, or for providing auditory stimulation for tinnitus and hyperacusis therapy. For example, noise with a large bandwidth (e.g., broad band noise, white noise or substantially white noise) or pure tones may be used as well and in certain embodiments, for example, white noise may be combined with, for example, music or other sounds (e.g., speech) to create a combined audio signal.

Additionally, certain embodiments described herein give particular emphasis to the use of conventional, insert or wireless headphone systems or insert type headphones in conjunction with a suitable personal sound reproduction system, such as a high fidelity personal music player (PMP) for audio cassette, CD, MP3, or WMA recordings. The cost of a high fidelity PMP may be around one-tenth the cost of, for example, conventional binaural maskers. However, it is to be understood that the tinnitus protocol disclosed may also be applicable to conventional hearing aid style devices, maskers and/or combinations thereof. The technique can also be applicable to the setting of additional user programs in hearing aids, or the modified signal may be transmitted to the tinnitus sufferer through their hearing aids' telecoil or induction coil facility. Additionally, conventional loudspeakers may be used in certain embodiments. However, in certain embodiments, use of conventional loud speakers may not be desirable to use in certain systems for reasons that will be apparent to persons of ordinary skill in the art based on this disclosure.

In addition, PMPs generally possess small headphones with long-throw transducers that enable far superior fidelity compared to most free field loudspeakers. Headphones are generally more effective than loudspeakers because they circumvent the extensive attenuation of high frequency sounds that occurs through a free field.

While developing an exemplary protocol, the required extended upper frequency stimulus presented challenges for the conversion of audiogram results to the required real ear response, given that there were no internationally agreed upon standards for the conversion between dB HL to dB SPL for 10 and 12 kHz pure tone and narrow band noise stimuli. Although there were no agreed upon standards at the time these conversions were initially required, today, the conversions may be provided for by, for example, the ISO-TR/389-5 standard, and in certain embodiments, this standard may be used for the calibration process.

The manufacturer's calibration specifications for a Madsen OB 822 audiometer were used to extrapolate the required values for use with a telephonics TDH 39 headphones and MX 41/AR cushions. The audiometer was professionally calibrated accordingly. The values for 10 kHz were 50 dB HL=59.5 dB SPL and at 12 kHz, 50 dB HL=61 dB SPL. All ISO hearing level frequencies below 10 kHz were calibrated as per the relevant Australian standards (AS 1591.2-1987). Table 1 lists the transfer/calibration values in inverted format used for converting dB HL to dB SPL.

TABLE 1

| | Frequency | | | | | | | | | | |
|---|---|---|---|---|---|---|---|---|---|---|---|
| kHz | 0.25 | 0.5 | 1 | 1.5 | 2 | 3 | 3 | 6 | 8 | 10 | 12 |
| dB | 25.5 | 11.5 | 7.0 | 6.5 | 9.0 | 10.5 | 10.5 | 16.5 | 12.0 | 9.5 | 11.0 |

A further feature of the this exemplary tinnitus protocol (TP1) developed by the inventors, was an adaptation of the so called “half gain rule”, whereby amplification for hearing loss compensates for only around one half of the hearing deficit. This rule underlies most current hearing aid prescriptive practices. The TP1 attempted to maximize the acoustic energy centered around the pitch of the individual's tinnitus, and to “balance” the headphone output to correct for any asymmetrical hearing loss. A further goal was to enable the balanced perception of the stimulus throughout the person's head, rather than at the ear level like traditional uncorrelated devices.

PMPs generally have a volume control range that exceeds what is available in hearing aids, and so the TP1 did not need to specify absolute gain figures. However, PMPs generally do not possess a left/right balance control, and this was expected to reduce their acceptability in cases of asymmetrical hearing loss and its associated loudness recruitment. As the TP1 formulae aimed to minimize the perceptual loudness of the music or noise or other acoustic stimulus required to provide some amount of relief of an individual's perception of tinnitus, it thus only needed to specify the relative frequency response characteristics for each ear when presented in those reproduction systems that do not provide individual control of each stereo channel. Maintaining discrete signals for each ear, and controlling the temporal correlation between those signals is advantageous because it allows any asymmetry in the auditory system disorders (e.g., levels of hearing loss) exhibited by the two ears to be accounted for. In this way, the degree of customization for the auditory system disorder is enhanced. This allows maximum stimulation in frequencies of hearing loss while at the same time ensuring a pleasantly low listening volume, thereby enhancing the listening experience for the user. By allowing a stereo, and hence a spatially distributed and more engrossing listening experience, user acceptability is further enhanced. A further advantage is that the integrative pathways of the auditory system are stimulated in this way.

The procedure for applying the TP1 was thus as follows:
(i) The individual's pure tone hearing level thresholds at each frequency were converted to dB SPL by the addition of the transfer values in Table 1.
(ii) The tinnitus pitch match frequency in the most severely affected ear was chosen for the maximal point of the base line calculation. The two adjacent best hearing thresholds of the lesser hearing loss ear was always chosen as the minimum point of the calculation. When a reliable pitch match was not found using pure tones, it was substituted with the mean of the two adjacent best hearing frequencies. Thus, the base line constituted a mid line value between the two greatest audiometric extremities.
(iii) The final equalization values were then derived by subtracting the base line from the hearing threshold (expressed in dB SPL) for each frequency and each ear. Thus the algorithm for patients whose tinnitus pitch could not be reliably determined was:

Baseline=0.5(A−B)+B

Required Equalization, REQ=0.5{SPL(0.25,0.5,1,2,3,4,6,8,10,12 kHz)−Baseline}

The algorithm for non-tonal tinnitus was:

Baseline=0.5(C−B)+B

REQ=0.5{SPL(0.25,0.5,1,2,3,4,6,8,10,12 kHz)−Baseline}
Wherein,
A=hearing threshold (dB SPL) at frequency of tinnitus pitch match.
B=mean dB SPL at the 2 adjacent least hearing loss frequencies.
C=mean dB SPL at the 2 adjacent greatest hearing loss frequencies.

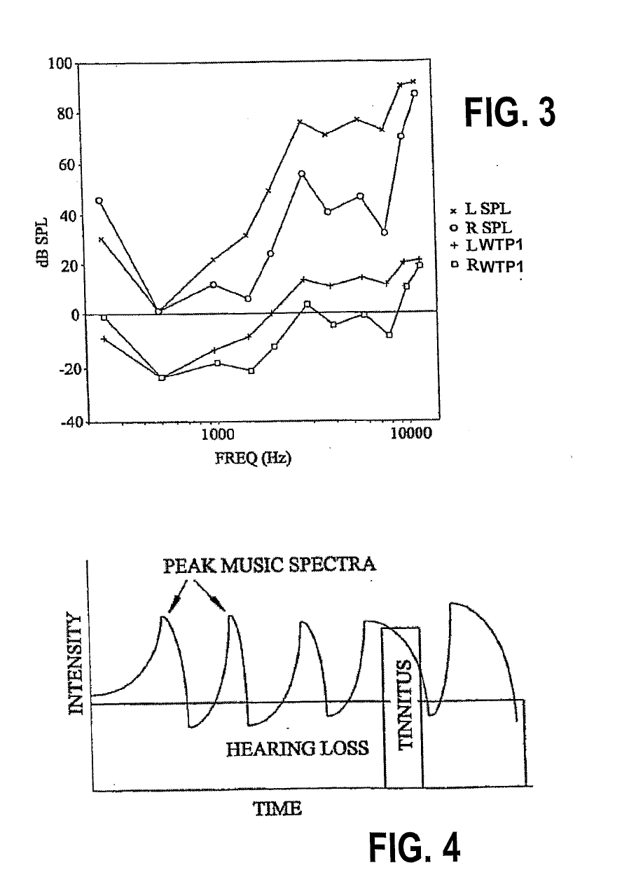

FIG. 3 is a graphical representation of an exemplary relationship between a typical individual's hearing levels, tinnitus and the TP1 “equalization curves”. As seen in FIG. 3, this individual has a steeply sloping high frequency bilateral hearing loss and tinnitus at 10,000 Hz, both greater on the left side. Consequently, the required equalization curves revolve around the equalizer's baseline, achieving a partial correction for hearing loss by boosting the amount of high frequency gain and also correspondingly attenuating the low frequencies. As the hearing loss and tinnitus is worse on the left, that ear receives correspondingly greater amplification. Because of the abnormal growth of loudness perception which usually accompanies sensorineural hearing loss, (recruitment, and/or the presence of hyperacusis), complete correction for hearing levels is not provided, as this may exceed the individual's loudness discomfort levels.

A tinnitus rehabilitation sound recording was then produced on an audio cassette tape for use in the individual's PMP. A stereo frequency equalizer (Genexxa 31-9082) was used in this procedure, which includes ten adjustable frequency bands per channel, with centre frequencies at 0.031, 0.062, 0.125, 0.25, 0.5, 1, 2, 4, 8, 16 kHz. Each control had a range of + or −12 dB SPL. The equalizer featured an “EQ record” facility, so that the audio signal could be passed through the equalizer circuit before being recorded. The equalizer's controls for each of the ten frequency bands was set to the calculated values for the left ear in the left channel of the equalizer, and the right ear values set in the right channel, in accordance with the particular individual's equalization values as calculated by the TP1 algorithm. The stereo output from a broadcast quality cassette recorder (or, in certain embodiments, any type of audio visual storage medium) was connected to the stereographic equalizer, which then had its output routed to another high fidelity cassette deck (or, in certain embodiments, any type of audio visual storage medium) for recording onto high fidelity audio cassette tape. Dual leads and stereo RCA connecters were used to preserve L/R channel separation in light of the advantages of a stereo signal with separately customized signals as described herein.

Modified sound recordings of both music and white noise were made for use in clinical trials with 30 participants. Each participant was counseled as to the rationale behind the therapy and the possible benefits of using the tinnitus rehabilitation sound recording. Each participant was issued a new PMP with standard insert headphones (Sony MDR E552) that fit into the concha and thus do not require a headband. Sound level-peak analysis measures were then performed. With their custom-made tape playing in the PMP, they were asked to slowly turn up the volume until they could just no longer perceive their own tinnitus. This level was marked on the volume control wheel. Each participant was told to notify the audiologist if they subsequently needed to turn up the volume further than the marked position. They were encouraged to experiment downwards with the volume control over the course of each session, as they might find that they require progressively less volume to provide relief if, for example, residual masking occurred.

One group of participants was given a noise tape whereas the other group was given a music tape. While both treatment groups had similar levels of pre-therapy distress associated with their tinnitus, the music group displayed a greater improvement by mid-therapy and these gains were maintained at a two-year post-therapy follow up. The noise group also displayed some improvement, but less dramatic then the music group. 96% of the participants found their music or noise tapes to be an effective device for providing relief.

In some cases, the TP1 appeared to present an unbalanced perception of loudness where the individual possessed a substantial inter-aural asymmetry. The real-ear perception of loudness may have deviated from the prescribed response due to perception of loudness differences at various points across the frequency range. It was also thought that the half gain rule for hearing aids might be best suited for the moderate hearing loss population, and that a mild hearing loss might only require one-third gain. Furthermore, it is possible that the recruitment of loudness phenomena might be greater in tinnitus patients than non-tinnitus patients, particularly given its high co-morbidity with hyperacusis and phonophobia (the fear of external sounds). These factors suggested that the TP1 might be over-compensating for hearing loss, and that further modifications were required to optimize the procedure.

An objective of the TP1 algorithm was to produce an acceptable substitute for the tinnitus at the lowest possible MML and to accommodate for any interaural symmetries. However, it was subsequently realized that an improved algorithm would be more robust if the prescription of the required equalization response was performed on the basis of maximum and minimum hearing levels, and thereby attempt to provide relatively equal sensation levels at all frequencies. Data from the TP1 study indicated that 44.4% of the music group, and 28.6% of the noise group participants preferred to set the volume of their audio tapes at a level which provided relief by only partially masking their tinnitus perception. This occurred despite being instructed that the optimal setting was to totally mask their tinnitus. The differences in masking level preferences between the two types of stimuli also suggests that music was more acceptable than noise when used at volume levels where the tinnitus could still be partly perceived. Whilst certain embodiments may totally mask tinnitus, other embodiments may partially mask. In fact, the present inventors have developed an improved tinnitus protocol based on providing relief via intermittent masking. Since music is a dynamic signal, it appears possible that the intensity of music which partially masks might actually constitute a form of intermittent masking. A schematic representation of intermittent tinnitus masking using a music signal is illustrated in FIG. 4.

It is believed that the providing relief of the perception of tinnitus with a relaxing stimulus (such as music) may be effective, by virtue of the distraction provided, on a psychological, as well as on an acoustic or neural level. It is feasible that providing relief with music might constitute a form of systematic desensitization. Whilst in a relaxed state, the listener might be alternatively perceiving, then not perceiving the tinnitus, according to the fluctuations in the peak levels of the music. The predictability of the music may mean that the tinnitus might not even be consciously perceived during the “troughs” of the music. Additionally, the tinnitus might “reappear” from the music often enough for habituation to occur. But the ongoing dynamic nature of the music signal prevents this limited exposure from being disturbing, and this may reduce any limbic system enhancement and/or conscious attentional focus on the tinnitus perception. Thus, the proposed relief (e.g., resulting from the intermittent-masking-with-relaxation-music technique) may promote a synergistic effect through its additional mechanisms of facilitating a sense of control, a reduction in general anxiety levels, and/or a form of auto-hypnosis leading to a reduction of fear about the tinnitus itself. Therefore, another exemplary algorithm based on a tinnitus retraining protocol (TRP) was developed that was designed to produce intermittent masking of the tinnitus.

In practice, the TP1 algorithm's use of the so-called half gain rule appeared to over-compensate for hearing loss as noted above, sometimes making the recording seem unbalanced or “tinny”. Conversely, there were several factors that suggested that one-third gain might not provide sufficient equalization. The long term music spectrum has considerably less high frequency energy than what is typically available from conventional tinnitus maskers, and yet the greatest hearing loss is typically concentrated in this region (see FIG. 1). Therefore, any substantial reduction of gain could prevent achieving adequate high frequency equalization to overcome the limitations in the music spectra and the effects of hearing loss. Therefore, because the so-called half gain rule was sometimes excessive, but one-third gain may be insufficient in certain situations for the purposes of modifying music for long term tinnitus retraining, a medium was selected by the incorporation of a 0.4 gain multiplier, (M). In certain embodiments the gain multiplier may be between 0.3 to 0.5, 0.0 to 1.0, 0.2 to 0.6, 0.1 to 0.7, 0.2 to 0.8, 0.4 to 0.9, about 0.25, about 0.30, about 0.35, about 0.40, about 0.45, about 0.50, about 0.55, or about 0.60.

To further facilitate the provision of equal sensation levels of music across the full spectral range of the music signal, the TRP algorithm adopted the ISO Equal Loudness Contours (ELC). The ELC transfer values correct for any differences in loudness perception depending on the discreet frequencies (International Standards Association, 1961). The 40 phon contour curve was selected because the earlier study found that the mean participant's customized music recordings, under total masking conditions, displayed a RMS of 45.7 dB SPL. Thus, with 8 dB representing an approximate doubling of perceived loudness, 37.7 dB was extrapolated to be the mid point between the threshold and total masking, and thus representative of the intensity around which intermittent masking would occur with those with a mild to moderate sloping hearing loss. The 40 phon contour was thus utilized because it was the closest to this mid point, and choice of the lower value curve also helped compensate for loudness recruitment.

One standard audiometric procedure is to obtain hearing thresholds using TDH 39 headphones, and the results are expressed in dB HL (Hearing Level). However, one convention for specifying hearing aid characteristics is to utilize dB SPL (Sound Pressure Level) values. Consequently, the hearing thresholds (dB HL) obtained in the 6 cm³ headphones need to be converted into dB SPL by the addition of the transfer values in Table 1.

These transfer values were then summated with the 40 phon contour values. The resulting transfer/calibration values are displayed in Table 2.

TABLE 2

| | Frequency | | | | | | | | | | | |
|---|---|---|---|---|---|---|---|---|---|---|---|---|
| kHz | .25 | .5 | .75 | 1 | 1.5 | 2 | 3 | 4 | 6 | 8 | 10 | 12 |
| Corrections | 23.5 | 7.5 | 5.5 | 7 | 6.5 | 7 | 5.5 | 2.5 | 16.5 | 21 | 16.5 | 13 |

The tinnitus retraining protocol (TRP) algorithm is a modification of the TP1 algorithm given above, and is as follows:

REQ=0.4{ELC+SPL(0.25,0.5,1,2,3,4,6,8,10,12 kHz)−Baseline}
Where:
Baseline=0.5 (A−B)+B
A=mean dB SPL at the two adjacent greatest hearing loss frequencies in the greatest hearing loss ear.
B=Mean dB SPL at the two adjacent least hearing loss frequencies in the least hearing loss ear.
SPL=hearing thresholds (in dB HL), converted to dB SPL.
ELC=Transfer values for 40 phon Equal Loudness Contours.

Alternatively, the patient's hearing thresholds may be obtained using ⅓ octave narrow band noises, and the gain multiplier (M) becomes 0.7 (or between the range of 0.5 to 0.95, 0.3 to 0.5, 0.4 to 1.0, 0.2 to 0.6, 0.4 to 0.7, 0.4 to 0.8, 0.4 to 0.9, about 0.40, about 0.45, about 0.50, about 0.55, about 0.60, about 0.75, about 0.9, or about 1). In certain embodiments the gain multiplier may be between about 0.3 to 0.5, 0.0 to 1.0, 0.2 to 0.6, 0.1 to 0.7, 0.2 to 0.8, 0.4 to 0.9, about 0.25, about 0.30, about 0.35, about 0.40, about 0.45, about 0.50, about 0.55, about 0.60, about 0.7, about 0.8, about 0.9, or about 1.0.

The procedure for applying the TRP was as follows:
(i) The person's audiogram was perused to ascertain the two adjacent greatest hearing loss frequencies in the greatest hearing loss ear (A), and also the two adjacent least hearing loss frequencies in the least hearing loss ear (B).
(ii) These four dB HL values were then converted to dB SPL by the addition of the transfer values in Table 1.
(iii) The dB SPL mean of the two adjacent greatest hearing loss frequencies in the greatest hearing loss ear (A) was then calculated in dB SPL, and the procedure repeated for the two adjacent least hearing loss frequencies in the least hearing loss ear (B).
(iv) A midline value was then calculated by the subtraction of B from A, which value is then halved, and the result added to the B value. This is the TRP baseline.
(v) All of the dB HL thresholds from the audiogram were then added to the values in Table 2 above which is the summation of the ISO 40 phon ELC correction values, and the dB HL to dB SPL transfer functions. This produces a measure of hearing in terms of the relative perceived loudness of stimuli at each of the discrete frequencies. The values were expressed in dB SPL so that the desired equalization frequency response could be determined within the 24 dB SPL range of the graphic equalizer.
(vi) The baseline value was then subtracted from each transformed threshold, and its result then multiplied by a 0.4 gain. This process is repeated for each frequency of each ear.
(vii) These values were then used to manually set the graphic equalizer with the left ear's required equalization response (REQ) used in the left channel, and the right ear's REQ used in the right channel of the equalizer.

The audiogram for the participant chosen to demonstrate how the TP1 accounts for a steeply-sloping asymmetrical hearing loss (see FIG. 3), was also chosen to demonstrate how the TRP algorithm modifies the intensity of the audio signal at selected frequencies to provide relief of the perception of the tinnitus. Tables 3 and 4 below show the calculations at each frequency for the left and right ears, respectively, using the TRP algorithm above. The baseline calculation was made as follows:

$$\begin{matrix}Baseline = 0.5 ( A - B ) + B \\ = [ 0.5 ( L SPL 10 + L SPL 12 ) - 0.5 ( R SPL 0.5 + R SPL 0.75 ] \times \\ 0.5 + 0.5 ( R SPL 0.5 + R SPL 0.75 ) \\ = [ 0.5 ( 89.5 + 91 ) - 0.5 ( 1.5 + 8.5 ) ] \times 0.5 + 0.5 ( 1.5 + 8.5 ) \\ = ( 90.25 - 5 ) \times 0.5 + 5 \\ = 47.625\end{matrix}$$

TABLE 3

| | | | Corrections and Calculations | | | | | |
|---|---|---|---|---|---|---|---|---|
| | | | ELC & | | | | | |
| | | | SPL | | | | | |
| | | | trans- | | | | | |
| L. | P's | | fer | | − | | | |
| Freq | dB | P's | funct- | | Base- | | | |
| (Hz) | HL | SPL | ions | = | line | = | ×0.4 | =REQ |
| 250 | 5 | | 23.5 | 28.5 | 47.625 | −19.15 | ×0.4 | −7.66 |
| 500 | −10 | | 7.5 | −2.5 | 47.625 | −50.15 | ×0.4 | −20.06 |
| 750 | 2.5 | | 5.5 | 8 | 47.625 | −39.65 | ×0.4 | −15.86 |
| 1000 | 15 | | 7 | 22 | 47.625 | −25.65 | ×0.4 | −10.26 |
| 1500 | 25 | | 6.5 | 31.5 | 47.625 | −16.15 | ×0.4 | −6.46 |
| 2000 | 40 | | 7 | 47 | 47.625 | −0.65 | ×0.4 | −0.26 |
| 3000 | 65 | | 5.5 | 70.5 | 47.625 | 22.85 | ×0.4 | 9.14 |
| 4000 | 60 | | 2.5 | 62.5 | 47.625 | 14.85 | ×0.4 | 5.94 |
| 6000 | 60 | | 16.5 | 76.5 | 47.625 | 28.85 | ×0.4 | 11.54 |
| 8000 | 60 | | 21 | 81 | 47.625 | 33.35 | ×0.4 | 13.34 |
| 10000 | 80 | 89.5 | 16.5 | 96.5 | 47.625 | 48.85 | ×0.4 | 19.54 |
| 12000 | 80 | 91 | 13 | 93 | 47.625 | 45.35 | ×0.4 | 18.14 |

TABLE 4

| | | | Corrections and Calculations | | | | | |
|---|---|---|---|---|---|---|---|---|
| | | | ELC | | | | | |
| | | | & | | | | | |
| | | | SPL | | | | | |
| | | | trans- | | | | | |
| L. | P's | | fer | | − | | | |
| Freq | dB | P's | funct- | | Base- | | | |
| (Hz) | HL | SPL | ions | = | line | = | ×0.4 | =REQ |
| 250 | 20 | | 23.5 | 43.5 | 47.625 | −4.15 | ×0.4 | −1.66 |
| 500 | −10 | 1.5 | 7.5 | −2.5 | 47.625 | −50.15 | ×0.4 | −20.66 |
| 750 | 0 | 8.5 | 5.5 | 5.5 | 47.625 | −40.12 | ×0.4 | −16.85 |
| 1000 | 5 | | 7 | 12 | 47.625 | −35.65 | ×0.4 | −14.26 |
| 1500 | 0 | | 6.5 | 6.5 | 47.625 | −41.15 | ×0.4 | −16.46 |
| 2000 | 15 | | 7 | 22 | 47.625 | −25.65 | ×0.4 | −10.26 |
| 3000 | 45 | | 5.5 | 50.5 | 47.625 | 2.85 | ×0.4 | 1.14 |
| 4000 | 30 | | 2.5 | 32.5 | 47.625 | −15.15 | ×0.4 | −6.06 |
| 6000 | 30 | | 16.5 | 46.5 | 47.625 | −1.15 | ×0.4 | −0.46 |
| 8000 | 20 | | 21 | 41 | 47.625 | −6.65 | ×0.4 | −2.66 |
| 10000 | 60 | | 16.5 | 76.5 | 47.625 | 28.85 | ×0.4 | 11.54 |
| 12000 | 75 | | 13 | 88 | 47.625 | 40.35 | ×0.4 | 16.14 |

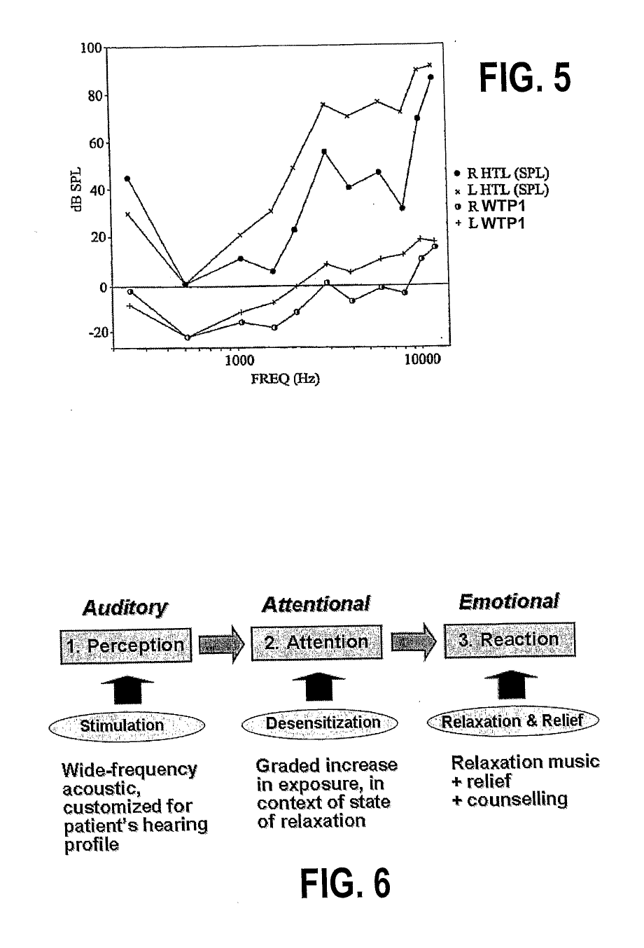

The REQ equalization curves for both ears are illustrated graphically in FIG. 5. A comparison of FIG. 5 with FIG. 3 will confirm that the patient's right and left hearing thresholds [HTL (SPL)] curves are identical.

In view of the results of the first clinical study and the additional understanding of the inventors, a second clinical study was conducted in which 90 people who suffer from tinnitus participated. The participants were allocated into one of four treatment groups: one group to test a second tinnitus protocol (TP2) which was intended to provide substantially complete masking, i.e., a high degree of relief and interaction, one to test the tinnitus retraining algorithm (TRP) which was intended to provide intermittent masking/relief/interaction, one to empirically measure the current TRT approach of using low-level broadband noise stimulation, and a quasi-control group to receive counseling alone. The second study exceeded expectations, with materially improved levels of habituation experienced by more than three-quarters of the participants using spectrally modified music. The adoption of bibliotherapy and TRT-style counseling resulted in significant improvements in clinical outcomes for all treatment groups. However, counseling alone appeared to be insufficient treatment for most participants. An important finding was that the TRP group experienced the greatest mean improvements in tinnitus distress. The TP2 stimulus group initially displayed a more rapid improvement, but the more gradual gains of the TRP group were sustained for longer, and ultimately were superior. There was little difference between the noise and counseling alone groups at post therapy and follow-up, although the mean improvements experienced by the counseling alone group were ultimately not statistically significant. While all treatment groups recorded mean reductions in tinnitus distress over therapy, the two music groups ultimately appeared to be the most effective. Approximately three-quarters of the music group participants experienced significant habituation to their tinnitus (TP2=78.6%, TRP=75%).

There were substantial reductions in ratings of reduced sound tolerance (e.g., hyperacusis) for both music groups, and a slight reduction for the noise group. The group without acoustic stimulation (counseling only) displayed deterioration in reduced sound tolerance ratings over the same period, strongly indicating that the provision of acoustic stimulation was a key ingredient in the reduced sound tolerance improvements. The music group participants often reported that their reduced sound tolerance levels tended to improve faster than their tinnitus perception.

The clinical studies therefore suggest that total masking with music may be more effective to facilitate a rapid improvement in distress and relaxation levels, despite the fact that intermittent masking/interaction with music eventually proved to be more effective on several measures. This indicates that a two-stage approach to acoustic stimulation might be most efficient, whereby patients initially should employ a total masking algorithm to give a stronger sense of relaxation, relief and control, then later switch to intermittent masking/interaction to remove the tinnitus detection. Maximizing the sense of relaxation, relief and control in the early stage of the treatment is desirable for many patients who may be in a severe state of distress prior to commencing treatment for their tinnitus.

Certain pharmaceutical agents may complement acoustic therapy by further aiding relaxation. For example, it is believed that administration of an anxiolytic agent, particularly of the benzodiazepine class, including agents such as alprazolam and diazepam may help the patient to achieve a relaxed state in the initial stages of treatment.

Following a period of listening to the stimulus in a manner which facilitates relaxation, relief and control, with or without concomitant use of pharmaceutical agents which promote relaxation, such patients may then proceed more comfortably into a treatment phase in which the tinnitus perception is exposed more fully in order to promote habituation or desensitization. Although the present disclosure is not to be limited by the following theory, it is believed that such a phased approach may facilitate habituation or desensitization to the tinnitus by providing the patient with a form of systematic desensitization. Within the context of a relaxation stimulus (i.e. relaxing music), the patient is presented in the successive phases as described above with a graded increase in exposure to the tinnitus perception during listening sessions. It is believed that this assists with retraining of the neurological processes relating to attentional focus on the tinnitus perception and the patient's emotional reaction to it.

Although the present disclosure is not to be limited by the following theory, it is believed that there are interrelated processes in the development of disturbing tinnitus (each of which is believed to involve, at some level, neuro-plastic changes in the brain). As illustrated in FIG. 6, these processes may be characterized as involving (1) changes within the auditory system which lead to the initial perception of the tinnitus sound, (2) the attentional filters in the brain which cause the patient to pay attention to the tinnitus perception, and (3) the emotional response and the autonomic nervous system which cause an adverse reaction to the tinnitus.

More specifically, with respect to initial tinnitus perception, it is believed that auditory deprivation causes the auditory system to become more active and more sensitive to sound. Following peripheral hearing damage, for example through noise insult or ototoxic drugs, there are changes in levels and nature of activity in the auditory nerves which appear to be centrally mediated. As a consequence, the auditory cortex receives altered neural input, which it interprets as sound. It is believed that the cortex detects the amplified background neurological activity, and interprets it as the sounds perceived in tinnitus. These changes in the auditory cortex may also involve reorganization of the tonotopic map. With respect to the awareness of tinnitus, it is believed that perceptual filters at work on all of the senses determine which sensory perceptions are brought to our conscious attention and which are not. These filters play an important function as they allow the brain to focus on what is important while preventing us from being overwhelmed by sensory input. These filters recognize specific patterns of neural activity, and are constantly being updated and refined through experience. In the case of tinnitus, an importance “label” is applied to the tinnitus sound, such that it is constantly brought to the patient's conscious attention. With respect to the emotional (limbic) and autonomic nervous system engagement, it is believed that the limbic system of the brain, which controls the patient's emotional state, and the autonomic nervous system, responsible for the so-called fight or flight reflex, become engaged in response to the awareness of tinnitus. This causes a stressful state of high arousal and anxiety in response to the tinnitus awareness, which has a significant impact on the quality of life and general well being. This reaction also reinforces the other two processes referred to above, i.e., it leads to further increases in the sensitivity of the auditory system, and reinforces the attentional filters. This in turn leads to further increase in tinnitus loudness and awareness, which in turn increases the level of stress, and so on, in a self-perpetuating cycle that can make the tinnitus progressively worse over time.

Accordingly, in view of this understanding, certain exemplary embodiments discussed throughout this disclosure address some or all of these understandings. For example, certain embodiments may deliver a broad frequency stimulus into the system to counter the need for increased auditory sensitivity due to auditory deprivation. The broad frequency stimulus may be spectrally modified to account for each patient's hearing loss profile and the modification may be performed separately for each ear and then the resultant stimuli may be combined in a manner that provides a balanced perception across the two ears, and is delivered in stereo to stimulate the integrative pathways of the auditory system as well as enhance the listening experience for the patient. In this way, the treatment stimulates as much of the system as possible, as evenly as possible, and thereby reduces the need for the brain to “turn up” the sensitivity in the auditory system.

Additionally, exemplary embodiments may use pharmaceutical agents which act to reduce neural activity, with the intention of lessening the increase in neural activity which results from the auditory system's response to auditory deprivation. These agents include, for example, anticonvulsants, particularly of the GABA agonist class, including vigabatrin.

Additionally, exemplary embodiments may use music, which aims to address the limbic system/autonomic nervous system involvement that causes the aversive reaction to tinnitus. This aspect draws on the belief that relaxation music is as effective as progressive muscle relaxation in generating a relaxation response, and is further reinforced by the relief and sense of control that comes from being able to shut out the tinnitus sound as well as by improvements in sleep that may result. All of these factors may lead to a reduction in the level of limbic system arousal and the consequential stress response.

Additionally, exemplary embodiments may use pharmaceutical agents which facilitate a relaxation response or a sense of relief or a reduced sense of arousal or anxiety. Other pharmaceutical agents which modulate the emotional response may also be used. These agents include, for example, antidepressants particularly of the SSRI class including fluoxetine, paroxetine and/or sertraline.

Furthermore, certain exemplary embodiments may address the attentional filters using the principles of systemic desensitization. That is, because of the dynamics of the music, once customized for the particular patient, the stimulus provides relief to the patient in the peaks of intensity in the music, while allowing the tinnitus to be momentarily perceived in the intensity troughs. By gradually increasing the degree of exposure to the tinnitus perception over time, the brain may be retrained to perceive the tinnitus sound but not to pay particular attention to it, and therefore, not to trigger a stress response to react to it.

Additionally, exemplary embodiments may use pharmaceutical agents which modulate the patients' ability to focus their attention, such as, for example, pyschostimulants, including dextroamphetamine and methylphenidate.

Since it is believed that processes involving neuroplastic change underlies the therapeutic changes which lead to reduced disturbance associated with auditory system disorders, exemplary embodiments may use pharmaceutical agents which facilitate neuroplastic change, such as, for example, anti psychotics including risperidone and/or olanzapine. Anticonvulsant agents, including carbamezapine and/or amitryptiline, modulate neuroplastic change and may also be used.

Generally, and for reasons detailed throughout this disclosure, exemplary solutions may consist of at least 2 stages: a 1st Stage and a 2nd Stage. In both stages, the acoustic signal is provided with pleasing and relaxing sounds making the treatment easy and pleasant to use. The 1st Stage typically provides an acoustic signal with a high level of interaction with the auditory system disorder to provide relief while using the treatment. The 2nd Stage provides an acoustic signal with a lower level of interaction with the auditory system disorder. In clinical trials, the “intermittent” exposure of the effects of the auditory system disorder during the 2nd Stage creates a desensitization process, and has proven to be efficient and effective at reducing awareness of the auditory system disorder and the associated disturbance. In clinical practice, it has been the inventors' experience that more than two phases of treatment may be advantageous for some patients. For example, in the event of a deterioration in tinnitus as a result of stress, noise exposure or other exacerbating factors, some patients may find it desirable to temporarily transition from an intermittent interaction phase to once again utilize a high interaction phase in order to reemphasize the relief and control aspects of treatment.

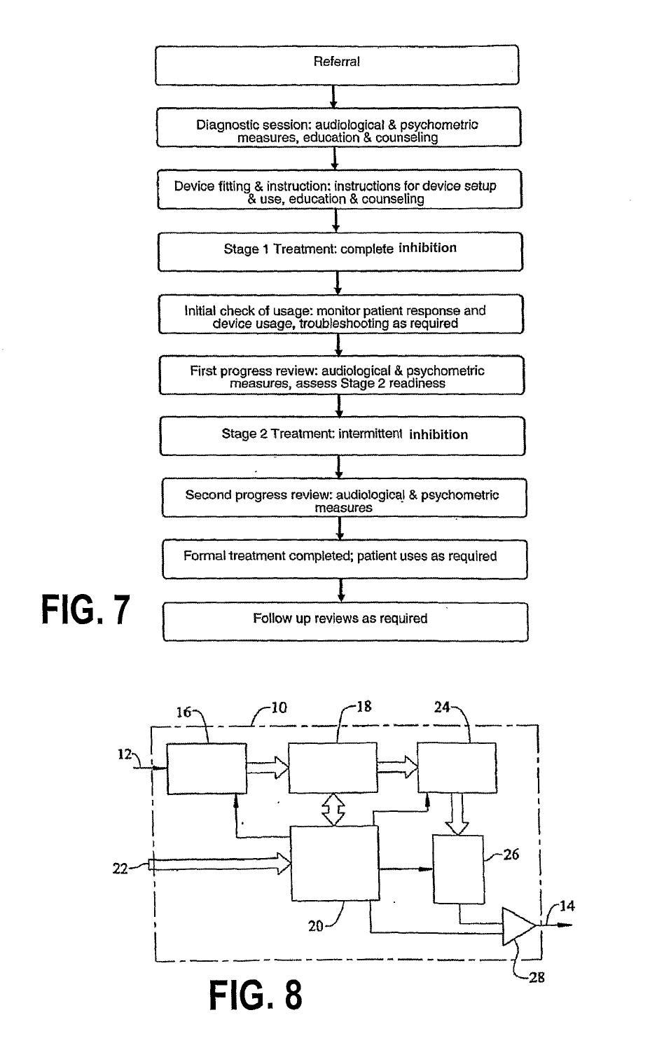

An embodiment of a 2 stage tinnitus rehabilitation method is outlined in FIG. 7. Following referral, for example from an Ear Nose and Throat specialist or other clinician, the process begins with diagnostic tests of the patient's audiological characteristics, as well as education as to the likely pathogenesis of their tinnitus. A tinnitus treatment device is then prescribed with embedded acoustic therapy customized for the patient (e.g., spectrally modified music plus added noise) and instructions provided for its use so as to provide complete masking (Stage 1 of the method). The patient's response is checked a short period (e.g., around two weeks) later and any difficulties that the patient may be encountering in using the device are discussed and resolved. After a further period of, for example, four to ten weeks (e.g., five, six, seven, eight, etc.), subject to patient readiness, the Stage 2 acoustic signal (e.g., spectrally modified music without added noise) is provided, with instructions for its use so as to provide intermittent masking/interaction and so greater exposure of the patient to their tinnitus. Progress review appointments include the measurement of key audiological and psychometric parameters in order to monitor progress and provide positive feedback to the patient. In addition, these appointments include a review of patient compliance, for which patient usage information has been logged and stored within the device for retrieval and review by the clinician. Throughout treatment, patients are instructed to adjust the volume setting on the device at the beginning of each listening session. During Stage 1 of the method, the volume is set so that the combined spectrally modified music/noise signal just masks the tinnitus. During Stage 2, the volume is set so that the tinnitus is masked during the musical peaks, and is momentarily apparent during the troughs; as patients become progressively more habituated to their tinnitus, the perceived level of tinnitus may decline over time, and accordingly, patients may need to set the volume progressively lower from session to session.

In the clinical studies, pre-recorded music was spectrally modified using the predetermined algorithms, and re-recorded on audio cassette tapes for participants' use. In light of copyright considerations, purchase of the rights to re-record music from selected recording companies or the commissioning of special purpose recordings may be required. In certain exemplary embodiments a programmable device for use by private practitioners may be provided. The device thus envisaged can be programmed by a qualified audiologist or health professional to account for each individual's tinnitus and hearing loss characteristics, using the tinnitus algorithms and clinical protocols described herein. In certain embodiments, the device may take the form of a musician's hearing aid type device designed to spectrally modify the audio signal as it enters the wearer's ears. Other exemplary embodiments may provide the device in the form of a device which can be employed in conjunction with a PMP and has an input adapted to connect to the audio output headphone jack on the PMP. The device would have a standard headphone jack to which a conventional PMP headphone can be connected. In an alternative exemplary embodiment, a modified sound recording is automatically generated in the audiologist's clinic, tailored to the patient's audiometric configuration, using software accessed via the World Wide Web.

FIG. 8 illustrates in schematic block diagram form an exemplary embodiment of a tinnitus rehabilitation device. The device 10 has an input 12 adapted to receive a two-channel stereo signal from the headphone output jack of a PMP. The device 10 also has an output 14 which provides a two-channel stereo signal, spectrally modified by a predetermined algorithm programmed into the device 10, which is suitable for listening to through a conventional PMP headphone. In certain embodiments, the device 10 employs digital signal processing, and therefore the left and right input audio analog signal is converted to digital format in an analog to digital converter (ADC) 16. The digital output signal of ADC 16 is then sent to a digital filter 18 which filters the digitized audio signal in accordance with a predetermined algorithm. The digital filter 18 modifies the intensity of the audio signal at selected frequencies in accordance with the algorithm.

The filter characteristic of the digital filter 18 may be programmed manually using thumbwheels or a similar interface or the digital filter 18 may be programmed electronically by means of a microprocessor-based controller 20 having a communications port 22 that may be connected to a desk top computer. Using a custom-designed software program which may accompany the device 10, an audiologist or other hearing aid dispenser can program the device 10 by means of a graphic user interface (GUI), which facilitates the input of the required clinical data into the non-volatile memory of the controller 20. Thus, for example, the clinical audiologist may simply enter the patient's pure tone hearing level thresholds at each of the 10 discrete frequencies from 0.25 to 12 kHz. The audiologist may also be required to enter the two adjacent least hearing loss frequencies (B) the hearing threshold at the frequency of tinnitus pitch match (A) and/or the two adjacent greatest hearing loss frequencies (C). Either the software or the controller 20 will then use these figures to calculate the baseline value, and employ the predetermined algorithm to calculate the required equalization values. These values are employed by the controller 20 to set the filter constants at each frequency in the digital filter 18.

The device 10 may also include an additional signal processing means 24, which is also under control of the controller 20, for providing further spectral modification of the digital audio signal after filtering by the digital filter 18. The spectrally modified audio signal is then converted back to analog format in a digital to analog converter (DAC) 26. An amplifier 28 may be provided to control the amplitude of the analog output signal provided at the output 14 of the device. It will be understood that each of the digital components of the device 10 may be integrated into a single integrated circuit, so that the dimensions of the device 10 can be made quite small and the device therefore remains inconspicuous.

In certain embodiments, the proprietary algorithms or digital processing of the audio signal may be entirely software-based, facilitating the production of a stored music medium (tape or compact disc or alternative medium in an encoded format, or using MP3, WMA or other coded format) for playback by the tinnitus sufferer on a standard personal sound reproduction system, such as a personal music player (PMP), with headphones. In this embodiment, the method of providing a tinnitus rehabilitation sound recording takes full advantage of the speed and economies provided by the Internet for fast digital communications and remote processing power. With no more than a desktop personal computer (PC) with CD-writing capability, the ability to provide a customized tinnitus rehabilitation sound recording can be placed at the fingertips of the audiologist, healthcare professional, or patient. By utilizing the reach of the World Wide Web and developing an application service provider (ASP), (also described as “on-line operating software”), the method can be extended to provide tinnitus relief and treatment to a global market.

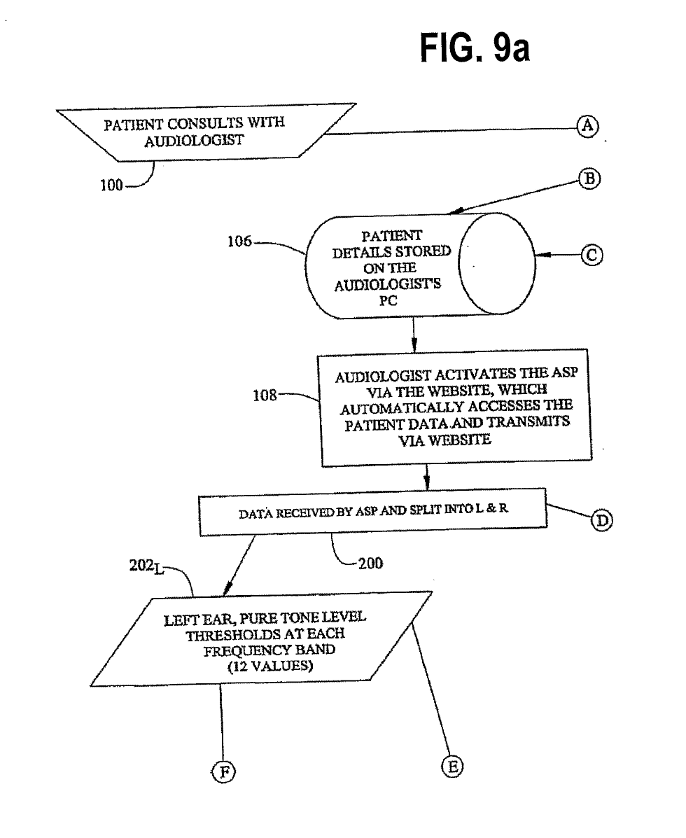

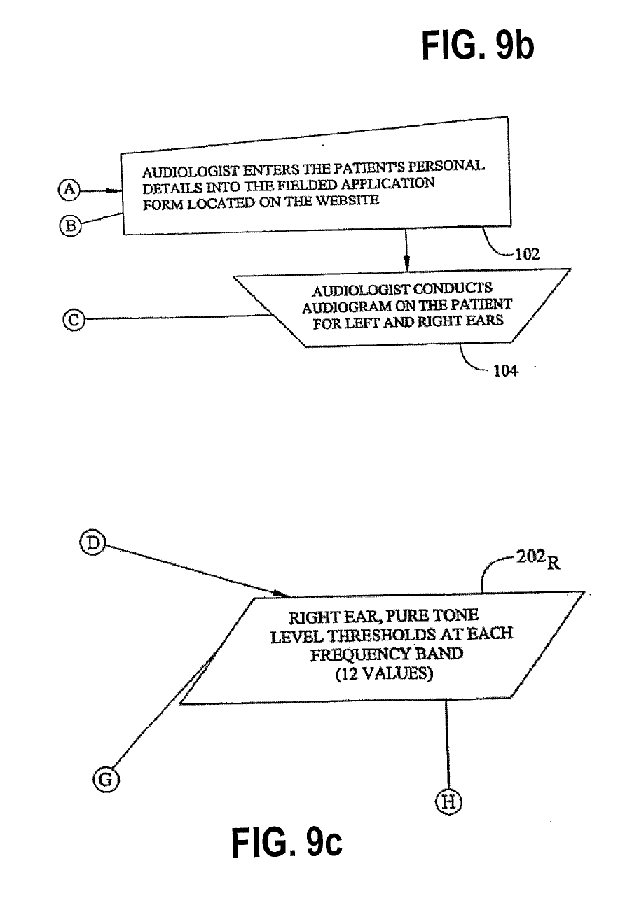

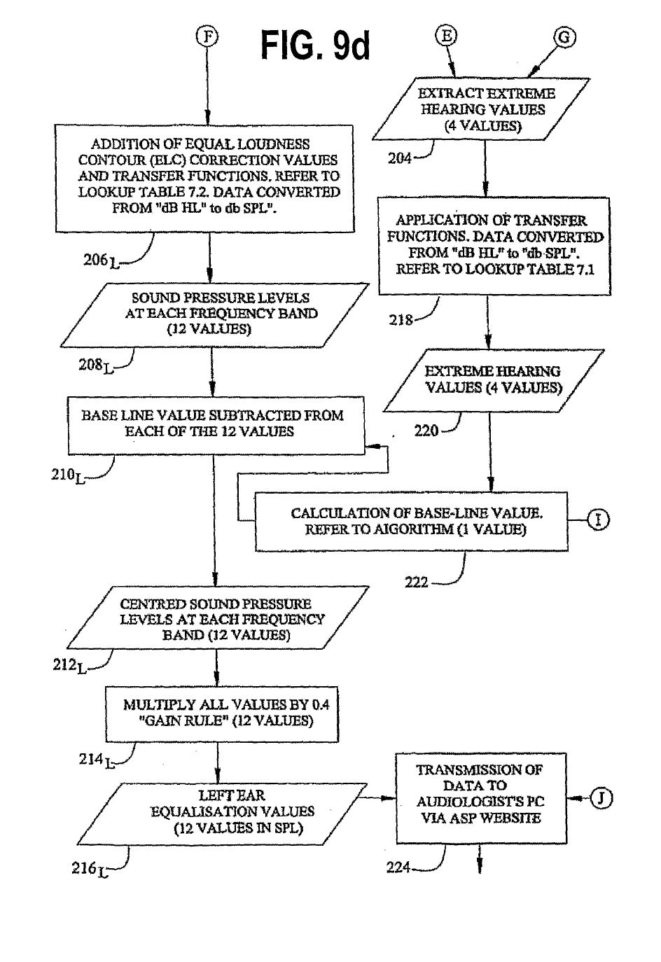

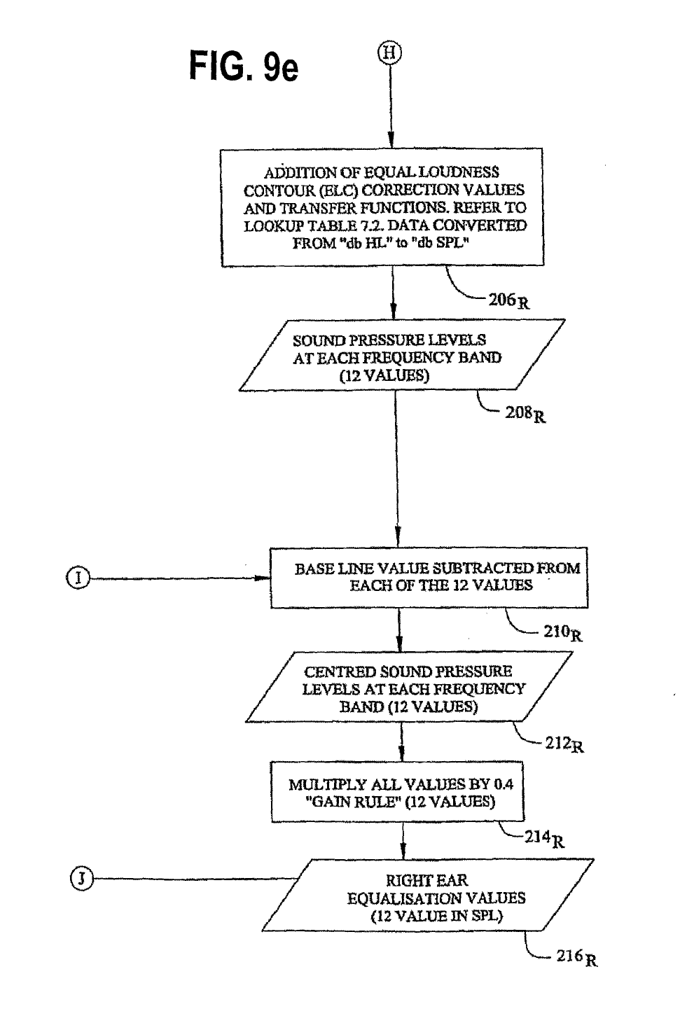

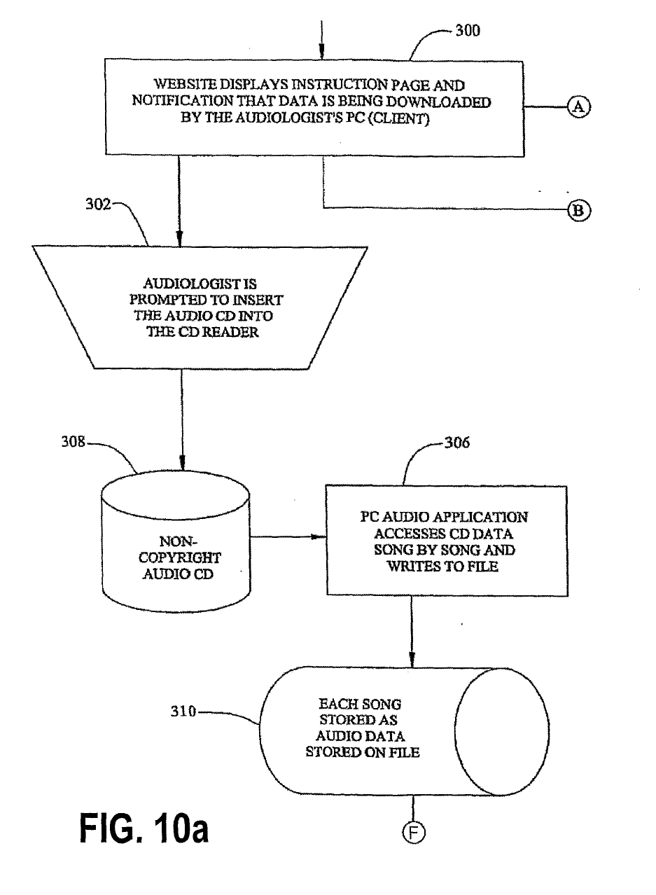

FIGS. 9 and 10 illustrate in flowchart form an exemplary method of providing a tinnitus rehabilitation sound recording utilizing the World Wide Web and the services of an ASP.

In this embodiment, the process commences in the audiologist's clinic (in other embodiments, the healthcare professional or audiologist may not be used) where the patient consults 100 with the audiologist. The audiologist enters 102 the patient's personal details into the appropriate fields in an application form located on a proprietary website. The audiologist then conducts 104 an audiogram on the patient's left and right ears. The audiogram is converted into an appropriate digital format and stored 106 on the audiologist's PC. The audiologist may then activate 108 the application service provider (ASP) via the website, which automatically accesses the patient data, including the digital audiogram, and transmits it via the website to the ASP.

Data is received 200 by the ASP and split into left and right ear processing channels. A central processing server (accessed via the ASP) houses the software containing the predetermined algorithms for converting the patient data to a digital filtering format herein referred to as a predetermined profile. This predetermined profile is then transmitted back to the audiologist's PC. The central processing server uses the digital audiogram to determine 202L, 202R the pure tone level thresholds at each of the predetermined frequencies for the left and right ears. The software ascertains 204 the two adjacent greatest hearing loss frequencies in the greatest hearing loss ear, and also the two adjacent least hearing loss frequencies in the least hearing loss ear. In each of steps 206L, 208L, 210L, 212L, 214L, 216L and 206R, 208R, 210R, 212R, 214R, 216R the tinnitus retraining protocol algorithm is applied to the left ear and right ear levels respectively, as is illustrated graphically in Tables 3 and 4 above.

In steps 218, 220 and 222 the baseline value is calculated, which is subtracted from each of the transformed threshold values for the left and right ears at 210L, 210R. The left and right ear Required Equalization Response (REQ) values are then transmitted 224 to the audiologist's PC via the ASP website. The website, which is visible on the audiologist's PC, notifies 226 the audiologist that the REQ values are being downloaded onto the audiologist's PC, and also prompts 302 the audiologist to insert a music CD (or any suitable media for storing the music or audio file such as (a DVD, memory card, etc.) into a CD player connected to the PC. The audiologist is also prompted 304 to insert a blank CD into the CD writer connected to his PC. It is to be understood that any suitable audio recording may be employed, preferably a music recording, stored on any suitable storage medium, such as a compact disc, audio cassette or MP3 card. Typically, the patient is offered a choice of music CD's, for which the appropriate copyright license fees have been paid, to be used as the base recording. An audio software application on the audiologist's PC accesses 306 the CD recording 308 and stores 310 the audio data to a file in the memory of the PC.

Proprietary software accessed by the ASP online reads the audio files stored in the PC and splits the signal into left and right stereo signals 320. Meanwhile, the REQ data received by the audiologist's PC is allocated 316 a channel reference (i.e. left channel data and right channel data 318). The software then converts 320 this left and right channel data into left and right predetermined profiles 322 respectively including interpolated values, between the twelve band frequencies, across the full frequency range up to, for example, 12 KHz. Software provided on the audiologist's PC accesses 324 and, using a (Fast Fourier Transform) FFT process (e.g., in a manner that would be readily understood by those experienced in signal processing in view of this disclosure), applies the predetermined profile to the right and left signals for the audio files in order to produce the left and right channels of the spectrally modified music signal 328. The modified audio files 328, one corresponding to each of the songs on the original music CD, are then utilized 330 by the CD Writer Software stored in the audiologist's PC, and are written to a blank CD 332.

The advantage of using an ASP and the audiologist's PC is that the amount of data transmitted and the processing power required by the server is in relative terms, very low. It is the processing of the audio signal that requires the bulk of the processing power. Via this model that power is housed in the PC of the audiologist instead of the server. Processing time would be negligible and therefore the entire process could be encompassed in the one patient visit.

Transmission is either via e-mail using a secure line with encryption or via a password-restricted web page; only qualified audiologists having access. Additional security measures such as ‘one-time-only-downloads’ or limiting the time the data is available on the website are also possible.

The consultation can easily be held in conjunction with a therapy session with the audiologist or healthcare professional, or as part of a coordinated therapy regime of on-going treatment. Certain embodiments may include the download of the proprietary software from the website and a royalty on each data download, i.e. for each CD made (not per patient, as each patient may wish to modify more than one CD). The Internet website could also provide a number of other services to assist in the relief of and treatment of tinnitus and hyperacusis. Thus, while music is one preferred embodiment, CDs can also be produced using noise, environmental sounds, pure tones, speech signals, or combinations thereof. It is also possible that parties could enter audiogram details without the help of an audiologist. When the audiologist or hearing aid dispenser does not have a CD burner, facility will be available for the CD to be produced at the ASP or other site, then posted to the clinic. As the data transmission speed of the Internet significantly increases, facility will be available for the processing of the audio signal to be performed within the ASP server if required.

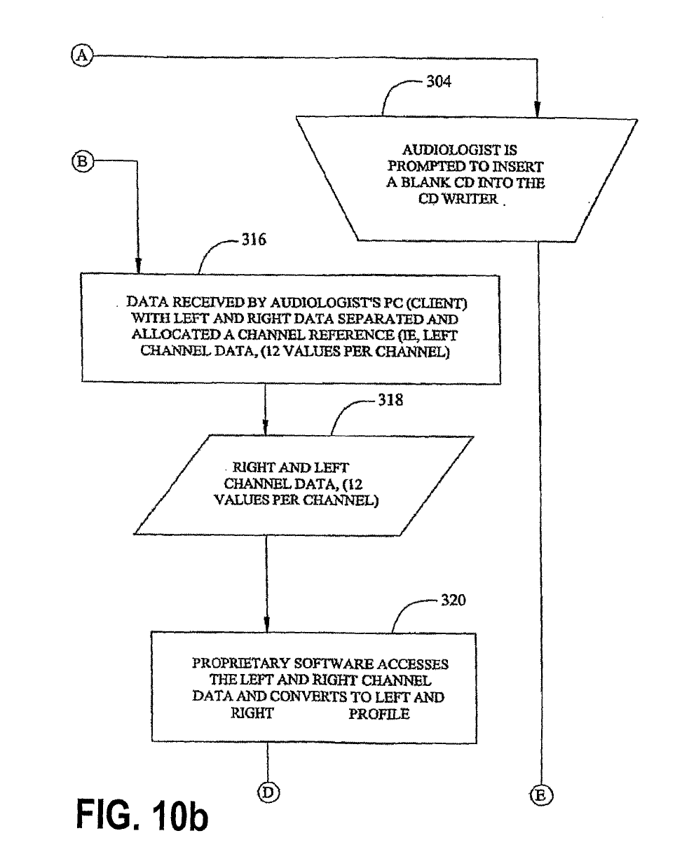

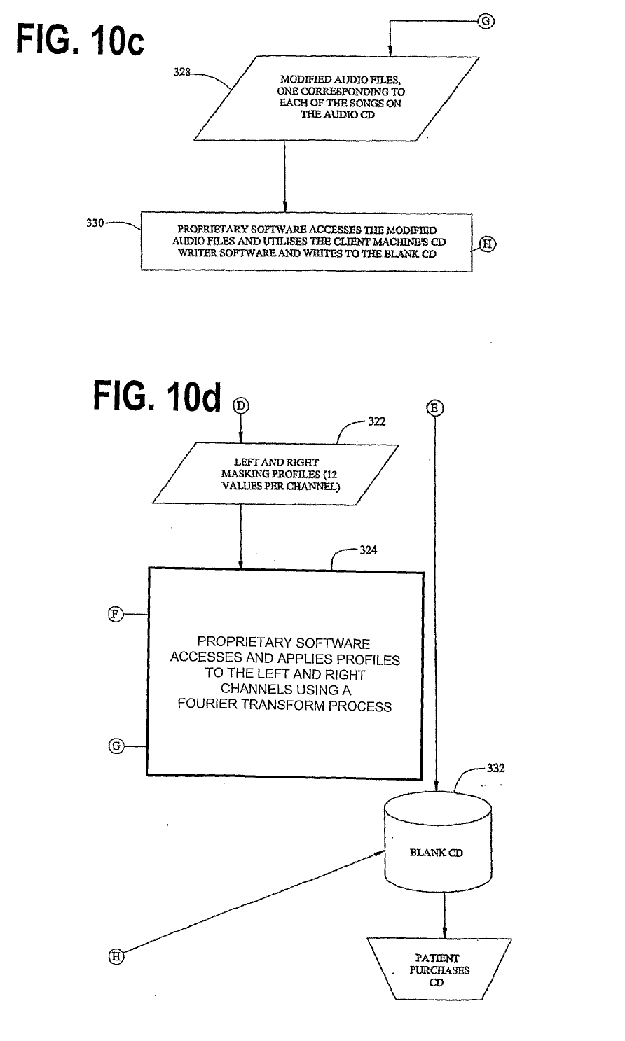

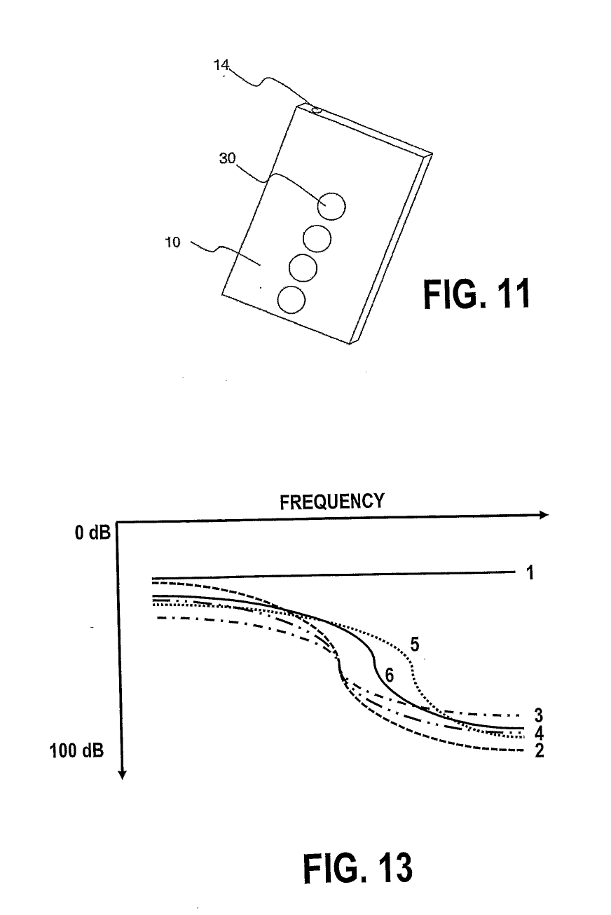

An exemplary embodiment of the tinnitus rehabilitation device 10 is shown in FIG. 11. The device 10 includes a stereo output 14 which may be used to connect the device 10 to headphones or earphones (not shown). Additionally, the device 10 includes a number of functional buttons for performing functions such as playing and stopping the audio signal, adjusting the volume of the audio signal and selecting whether to continue playing the same audio signal (replay function). Additional functions, such as those found on typical audio devices may also be added. As previously described, the device is in some embodiments small enough to be discrete and therefore, may include a battery. In certain embodiments, the battery may have a life that was sufficient to allow extended use (e.g., approximately one-week of use) without recharging. Also, as described above, the device may have a memory (e.g., a RAM card) for storing the audio signal such that an external media may not be required. In certain embodiments, a memory capable of storing sufficient audio signal to provide the patient with a choice of audio signals (e.g., approximately ½, 1, 2, 3, 4, 5, 6, hours or other time periods of listening time) may be used.

Additionally, a volume control may be provided such that the patient can set the volume of the audio signal to an appropriate level. In certain embodiments, the volume function would reset to a minimum value at the beginning of each treatment session. In this manner, the patient may be required to adjust the volume to either, in Stage 1 of the method, fully mask, or, in Stage 2 of the method, intermittently mask their tinnitus, the perceived level of which, as described above, may vary between sessions. This may be advantageous for ensuring that patients set the volume to the level that is most appropriate, substantially appropriate, or more appropriate at each listening session, rather than leaving the level as it was when previously used. However, in certain instances, it may be appropriate to leave the level as it was when previously used. For example, in some embodiments, the volume may be reset to a zero value or to a level that is just audible by the patient. In some embodiments, the processor may be configured to perform the volume resetting function. In some embodiments, the volume may be reset to a value that is determined by a patient's particular profile (e.g., the patient's audiogram).

In an embodiment of the device 10, the audio signal may be outputted from an internal storage medium, onto which the customized signal has been stored. In an alternative embodiment, the filtering means is incorporated within the device such that it acts “on the fly” to modify any input signal to generate a customized output signal. In this embodiment, the device 10 may also include a safety locking mechanism which prevents the outputting of any signal that does not include a specific coding which denotes that it has been appropriately modified so as to be appropriate for use by that patient. In certain embodiments, the filtering means might be incorporated within the device but the device may preprocess the input signal which is stored in the device 10 to create and store the customized output signal or a part of the customized output signal. This configuration may be beneficial in embodiments where the processor within the device is not fast enough to act “on the fly” or where the battery life of the device was limited and processing would diminish the battery life prematurely.

Although many of the exemplary embodiments discussed herein use an audiologist to take a patient's full audiogram and use that patients data to create a profile which is used to produce the spectrally modified music signal, neither the audiogram or audiologist is required in certain embodiments.

In certain embodiments, a self administered audiogram approximation may be provided and the resulting data may be used to create a predetermined profile which is used to produce the spectrally modified music signal.

For example, in certain embodiments, tones or bands of noise or tone combinations may be provided to the patient and the patient may turn up volume of the tone until it is audible. In this embodiment, the patient may be provided with these tones in a clinical setting or in a non-clinical setting. If provided in a non-clinical setting, the tones may be provided to the patient over the internet or at a kiosk in a commercial environment or by the device. The user may interact with an interface provided and based on the user's selected volumes, the system may generate a predetermined profile for the patient and, in certain embodiments, provide the profile to the patient. Alternatively, the tones may be provided with random amplitudes and frequencies and the patient may simply be required to indicate whether the tone is audible or not. In certain embodiments, this type of a process may be more reliable than allowing a patient to select appropriate volumes since it may allow for the creation of a more accurate audiogram approximation. In some embodiments, ranges of tones or multiple tones may be provided simultaneously.

In other embodiments, a graphic equalizer interface may be provided to the patient to allow the patient to select his/her preference. In this embodiment, the equalization interface may be provided on the device itself or alternatively, may be provided over the internet, in a kiosk, or other suitable means.

In certain embodiments, once the audiogram approximation is obtained, the appropriate data may be provided to the patient to load onto the device (e.g., the PMP device) so that the device can create the modified signal “on the fly” or the data may be stored with a provider (e.g., an entity that provides services or devices related to auditory system disorder rehabilitation) so that the audio signals can be modified by the clinician, other healthcare professional, or retailer before providing a modified audio signal to the patient.

In certain embodiments, a set of standard/generic profiles may be provided and a selected profile could be used to create a predetermined profile which could be used to produce the spectrally modified music signal or, in certain embodiments, the selected profile may be used to directly create the modified audio signal. For example, in certain embodiments, the patient may be able to select one profile from a number of predefined profiles. This selection may occur at the device through a user interface or may be provided in a clinical or commercial setting, including over the internet. In certain embodiments it may be desirable to provide a predefined set of profiles that are accessible in a structured fashion. This may be desirable if, for example, there is an unmanageably large number of possible profiles or if it is desirable to select the profile for the patient instead of allowing the patient to arbitrarily select a profile. In this embodiment, the patient may select a profile from a number of profiles based on a defined interface or logical structure (e.g., a hierarchical tree or decision tree) for determining the best profile. For example, the interface might ask the patient to answer a series of questions, or alternatively or in combination, may use one of the other methods described herein (e.g., providing random tones) to select an appropriate profile (e.g., in certain embodiments, the system may use one of a number of predetermined profiles based on the audiogram approximation discussed above).

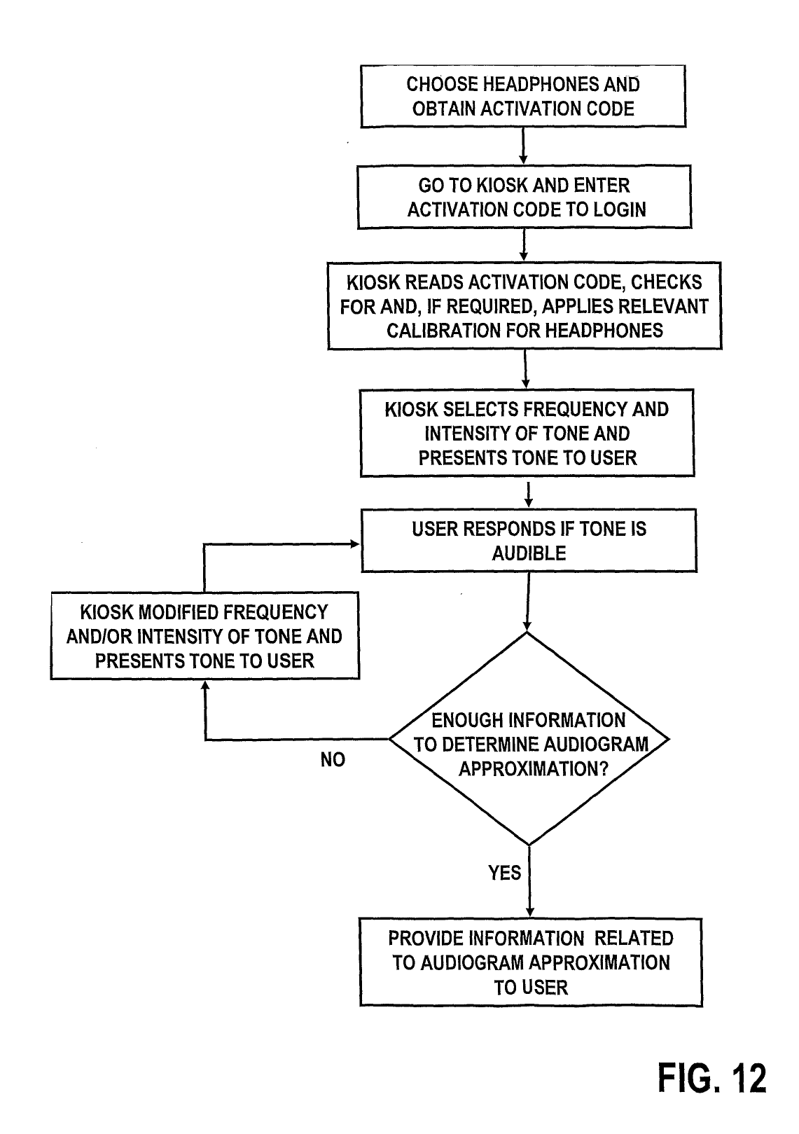

An embodiment for an exemplary method is illustrated in FIG. 12. As shown in FIG. 12, a user may arrive at a pharmacy or other desirable location and select headphones and obtain an activation code from a pharmacist or other third party. The activation code may include calibration information specific to the headphones in use. With the activation code and headphones, the user goes to a kiosk and logs into the kiosk to obtain a profile. The kiosk selects a particular frequency and/or intensity of a tone and presents the user with the tones via the headphones to individual ears and the patient responds when the tone is audible. This process is repeated until the kiosk has obtained enough information to generate a profile or audiogram approximation of the user. In certain embodiments, the kiosk may seek information at frequencies including, but not limited to, 0.25, 0.50, 1, 2, 3, 4, 6, 8, 10, and 12 kHz. The kiosk may select to test these frequencies by providing tones to the user at random or in a systematic manner. Alternatively, the kiosk may be programmed to zero in on the frequency of hearing loss by measuring the difference in thresholds and obtaining more measurements in the determined frequency range. For example, in certain embodiments, the kiosk may determine that there is hearing loss between 8 and 10 kHz and then seek further information at, for example 9 kHz; if there is no hearing loss at 9 kHz, the kiosk may then seek information about the users hearing threshold at 9.5 kHz.

Once the kiosk has the information required, it may provide the user with the necessary information for programming a corresponding device, or alternatively, may provide the information to a third party so that an employee can load the appropriate information onto the device.

In certain embodiments, the system may provide at least one profile that is adjustable by the patient or the clinician. The adjustment may be made using any combination of knobs, sliders, and or buttons on the device to adjust the low end and high end of the amplitude and/or the inflection frequency. The number of adjustments and/or the extent of the adjustment made can vary, and typically will vary, depending on the patient and/or the treatment being provided. In some aspects, the number of adjustments and/or the extent of the adjustments made may be at least 1, at least 2, at least 3, at least 4, at least 5, at least 6, at least 7, at least 8, or more depending on the particular circumstances. More specifically, and with reference to, for example, FIG. 13 (line 6), three adjustments could be made by a patient to adjust the modification provided to the audio signal. The patient may adjust the low end gain (left side of graph in FIG. 13), the high end gain (right side of FIG. 13), and the frequency at which the low end and high end are divided (i.e., the area frequency region in which the slope of hearing loss is steepest (e.g., the inflexion point)). The adjustments may be a continuous adjustment or may be incremental. Additionally, in some embodiments, the adjustment may be possible while the patient is listening to the audio signal. This type of an adjustment may be beneficial to modification prior to use in situations where the original audio signal is diverse (e.g., an audio signal from a television program, as discussed elsewhere herein).

Another embodiment of an exemplary method is described with reference to FIG. 13. In FIG. 13, the user is presented with multiple profiles and asked to select whether the currently presented profile is better or worse than the previously presented profile. In this embodiment, the selected profile may be used to modify the audio signal, that is, it may not be necessary for the system to obtain an audiogram or audiogram approximation. As shown in FIG. 13, the system presents the user with a first profile (1) (e.g., an audio signal modified by the first profile) as a base line and then presents a second profile (2) to the user and asks whether it is better or worse (based on the user's perception of relief (e.g., more or less relief) or some other benefit perceived by the user) than the baseline. Based on that response, the system provides a third profile (3) to the user and asks the same question of the user. By selecting various ratios of loudness between low and high frequencies and various frequencies associated with hearing loss, the system can identify one of a plurality of profiles that satisfies the needs of the user. As seen in FIG. 13, the difference between profile (2) and (3) is the loudness ratio and the same is the case of profile (3) and (4). Once the ratio is identified, profiles (4), (5) and (6) each adjust the frequency associated with the hearing loss.

As discussed, the audiogram, audiogram approximation, and/or predetermined profile could be generated independent of the device (e.g., clinic or at a commercial location) and a predetermined profile could be provided in a format that was appropriate for a number of devices (e.g., an existing PMP device). In certain embodiments, this may be distributed to the user as a software application (possibly over the web). For example, the software could be installed onto a patient's personal computer and used to generate the audiogram approximation or select a predetermined profile. Once this information is obtained, the software may also modify the audio signals to create the modified audio signals based on the predetermined profile so that the modified audio signal could be loaded onto a commercially PMP device.

In certain embodiments, such as the exemplary embodiments where the patient is presented with tones, it may be beneficial for the patient to utilize the same headphones as those that will be used during treatment. This allows the patient to achieve an appropriate adjustment for the headphones (e.g., a type of calibration). This may be beneficial since headphone frequency response can vary and optimal results using one set of headphones may not be optimal for another set of headphones. Therefore, by utilizing the same headphones when obtaining patient data and during treatment helps to ensure that the treatment is as optimal as possible. As discussed elsewhere herein, it may also be beneficial to include some headphone calibration information in the computation of the patients hearing loss (for example, when the user purchases the headphones an activation code and calibration information may be provide to the user).

As described throughout this disclosure, in certain embodiments, the acoustic signal may be a music signal. Music signals generally cover a wider spectrum of frequencies and are often considered enjoyable to listen to, but other types of sounds may be used, as well as combinations of different audio signals. The signal may be spectrally modified in a clinical setting. If the clinic was to use music signals, the clinic may be able to record several hours of spectrally modified signals but it is likely that whatever the amount was the listening experience by the individual would begin to be repetitive. For several reasons, it may not be possible for the clinic to create the necessary diversity that individuals may be seeking.

Generally, in certain embodiments, it may be desirable to have an acoustic signal that has a broad frequency, is relaxing enough but rich and distracting enough to reduce perception, has a dynamic intensity over time, is pleasant enough to listen to, and is customizable. Accordingly, in certain embodiments, acoustic signals from the television, radio, etc. might be used. It is also possible that the addition of video may create additional relaxation and distraction for the patient and may also improve compliance with the treatment procedure in certain embodiments.

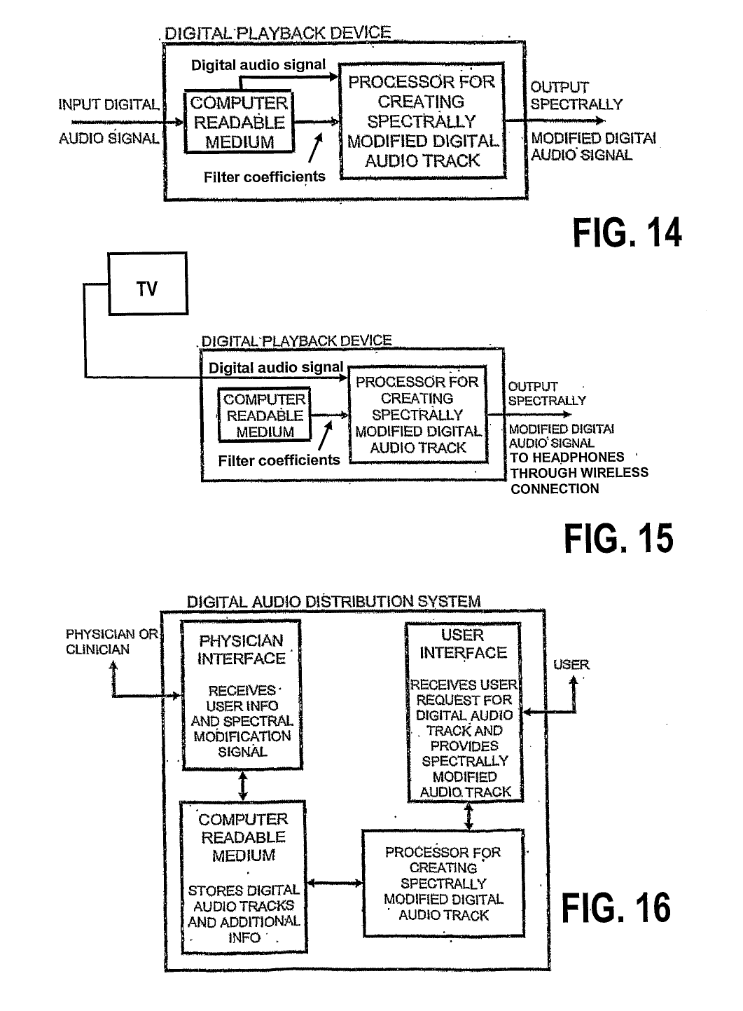

FIG. 14 is a functional schematic of a digital playback device in accordance with certain embodiments. As illustrated in FIG. 14, the digital playback device includes an input, a memory, a processor, and an output. The digital playback device may be a specialized device or in certain embodiments, the digital playback device may be a commercially available device such as an iPod (MP3 player), or some other device, such as, for example, a set top box for a television. Generally, commercially available devices may include players of any format. Some common audio formats, for example, include, but are not limited to, MP3, WMA, WAV, MP2, RA, MPEG, and many other equivalents. It should be readily understood by a person of ordinary skill in the art that the present inventions should not be limited to any specific set of file formats. These commercially available devices have become common because of their versatility, ease of use and inexpensive nature. In certain embodiments, the digital playback device may include an input for inputting an audio signal, a computer readable medium for storing the audio signal, a processor for receiving the audio signal and modifying the filtering coefficients (also referred to as a spectral modification signal and it should be readily understood that an actual signal such as an audio signal may be used or alternatively, an algorithm may be used to modify the digital audio signal and that regardless of whether the signal or the algorithm is used, the digital audio signal can be modified independently for the left and right channels, as discussed previously) used in the digital playback device to produce a spectrally modified audio signal; and an output for outputting the spectrally modified audio signal. In this embodiment, the audio signal is stored in its original format and then modified by the processor before playback (e.g., in real time or substantially real time). In some embodiments, the modified signal may also be stored in the digital playback device. Additionally, the modification of the filtering coefficients can be performed in numerous ways to reduce the effects of an auditory system disorder and/or to make the digital audio signal more enjoyable for an individual with or without an auditory system disorder. Specifically, instructions to modify the signal can be programmed into the processor from the factory, it can be loaded into the processor by a user or a clinic as software or firmware, or it can be implemented in hardware. In some embodiments, it may be adjustable by the user with controls on the device.

For example, and as discussed elsewhere in more detail, if the program is loaded from the factory, there may be different types of devices available. In one embodiment, for example, there may be two “models” for a device, a first model for spectrally modifying the audio signal for an individual with hearing loss at higher frequencies and increased sensitivity of loud noises at lower frequencies (See, e.g., FIG. 2) and a second model for modifying a signal for an individual with hearing loss at lower frequencies and sensitivity at higher frequencies. Of course, this is only an example and it should be readily understood that many different implementations could be effective, such as any of the exemplary embodiments for creating an audiogram, an audiogram approximation, or the use of a predetermined profile. In some embodiments, the digital playback device may also have a decoder for decoding the digital audio signal. For example, many commercial devices are able to play MP3, WMA, or equivalent files. In this case, the files are encoded in a specific manner and a special decoding device is generally utilized. If the decoding processor is provided, it is contemplated that the processor for spectrally modifying the digital audio signal may be incorporated in the decoding process or it may be provided separately. In situations where the processor is separate, the processor may be internal to the digital playback device or, in certain embodiments, may be coupled to the device externally. If the processor is coupled to the digital playback device externally, it may, for example, be coupled to the output of the digital playback device so that the processor can spectrally modify the digital audio signal before the spectrally modified digital audio signal is delivered to the user. In fact, an externally coupled device may allow the processor to be more readily programmed for the individual.

In certain embodiments, the device may include a compliance monitor for allowing a user to monitor how much time the user has used the device since it is generally important to use the device for predetermined amounts of time to be most effective. As with many digital playback devices, a battery for supplying power to the device is generally provided with sufficient battery life to allow extended use of the digital playback device without recharging. In some embodiments, the battery life may be at least 4 hours or as much as one week of regular use.

Additionally, since the digital storage technology improves daily, in certain embodiments, the computer readable medium for the digital playback device may be large enough to provide a diversity of audio signals. In certain embodiments, the computer readable medium storage capacity may be approximately equivalent to about 4 hours of the treatment signal or approximately 250 megabytes of capacity. Of course other capacities may also be desirable, for example, some device may have 20, 30, 40, or 60 gigabytes of capacity, and sometimes even more, while other devices may have as little as 10 megabytes of capacity or even less. The present inventions do not have to be limited to any specific capacity or capacity range.

In addition to playback, in certain embodiments, the digital playback device may include other advanced features. For example, since it may be important in certain embodiments to restrict the spectrally modified music to the individual for which the digital audio signal was modified, the digital playback device may include a user identification code in order to allow correct identification of the individual's own digital playback device in the event that more than one digital playback device gets placed together. Alternatively, so that the device may be shared, an on/off type of a switch may be used so that the digital playback device can be used without spectral modification. In another embodiment, the device may include a more advanced switch capable of spectrally modifying a digital audio signal in several different ways to accommodate several different individuals that may be sharing a device.

In certain embodiments, the digital playback device may also include a data downloading function for downloading logged information from the user device. Some information that may be useful, based on experience, is the times the device was used, the volume level of the device, what audio signals the individual listened to, etc.

The disclosed embodiments may be implemented in several ways. For example, the input on the digital playback device or the data downloading function described herein may be performed by a number of wired interfaces, infrared interfaces, or wireless interfaces.

In certain embodiments, the digital playback device may include a microphone as its input or as an auxiliary input. In this embodiment, addition of a microphone may be especially useful in, for example, a theater or for television, radio or similar setting. The signal input from the microphone may be stored on the computer readable medium or it may be processed and output to a user without storage. In either situation, the spectral modification in “real time” or substantially “real time” situations may add additional benefit to individuals with auditory system disorders that they may not otherwise have. Such a feature may be an addition to a conventional/commercial device such as an MP3 player or it may be part of a purpose built device.

In certain embodiments, the digital playback device with a microphone or other analog or digital input may be used in conjunction with a television or other audio visual source (which may provide a higher compliance rate than other methods). In this embodiment, the device may be similar to the device illustrated in FIG. 2, in that the device may include an input, a processor for creating a spectrally modified audio signal and an output for outputting the spectrally modified audio signal. The input may include any suitable input for receiving an audio signal from a television (e.g., any analog or digital input). Some exemplary inputs may include, for example, a microphone, a stereo wired input (e.g., RCA connectors), or a wireless input, such as a Bluetooth wireless connection or the like. The device may also include a computer readable medium, but such a medium may not be required since, in certain embodiments, it may be desirable to process the incoming audio in a substantially “real time’ basis. The processor could be any suitable processor for spectrally modifying the audio signal such as any of the embodiments discussed throughout this disclosure.

For example, the processor could be implemented in software or in hardware as an infinite impulse response (IIR) filter or a finite impulse response (FIR) filter. The IIR filter may be for example, a 5^{th} or 6^{th} order filter and the FIR filter may perform a convolution covering, for example, between 24 and 256 samples depending on the complexity of the response required (e.g., 24 samples, 48 samples, 50 samples, 55 samples, 64 samples, 100 samples, 128 samples, 256 samples, etc). Additionally, there may be one or more filters per channel and the accuracy of the filter may be between ±2-±20 dB (e.g., ±3 dB, ±6 dB, ±10 db, ±15 dB, ±18 dB, etc.). The filter parameters (or audiogram for calculating the filter parameters) may be, for example, loaded onto the device in a RAM or ROM type memory which may be integral with the device or may be removable (e.g., a card or similar device). In embodiments where music is loaded into the device with a removable memory card, it may be beneficial to load the filter parameters on the same card. The filter parameters may be automatically downloaded from the card to the processor to facilitate use of the processor with music or other stimulus from other sources. Additionally, in embodiments where a removable card is used, it may be possible to use the card as a user profile such that it could be inserted into a number of devices (e.g., a PMP and a set top box).

In some embodiments, the television audio may be combined with noise (e.g., broad band noise, white noise or substantially white noise) to further modify the input signal.

Since the television audio signal may be less predictable than, for example, music (e.g., music distributed by a clinician), in certain embodiments, it may be beneficial to provide additional modification to the television audio (or, in certain embodiments, other audio such as patient selected music). Examples of other types of modification may include, for example, compression of taller intensity peaks and/or, partial modification if part of the audio signal is not conducive to appropriate modification.

In certain embodiments, the device may be a personal and portable device as discussed throughout this disclosure. In certain embodiments, the device may be a set top box that is coupled to the television, receiver, or radio. The set top box, may include a wired or wireless output for connecting headphones (as described above) to the set top box (alternatively, the set top box may be an intermediate component that is coupled between a series of devices and the connection to speakers and/or headphones may be with the television or other device). Additionally, in certain embodiments, the device may be integral with the television or associated component or may be implemented in software or firmware and loaded onto the television or associated component. In addition to other advantages described herein, embodiments in which the device is coupled to an audio or audio visual device, such as a television or radio, provide an additional benefit in that usage of the device for relief from the auditory system disorder can be more easily integrated into the user's daily activities (which may ordinarily include periods of listening to, for example, radio or television). In this way, compliance to treatment is facilitated, and hence potential for benefit from treatment further enhanced. In certain embodiments, the device may apply somewhat different factors and/or algorithms when modifying audio signals which are derived from different sources in order to account for the different properties of those signals. For example, it may be desirable to apply a different ‘M’ factor or different levels of attention (e.g. by compression) of intensity peaks when processing an audio signal from a TV, radio or other ‘non-controlled’ source in order to reduce the risk that the patient is exposed to uncomfortably loud intensity peaks or other unpleasant audio transients.

An embodiment of an exemplary system is illustrated in FIG. 15. As seen in FIG. 15, the device is connected to the television via an appropriate wired connection and then a wireless connection is established between the device and a set of headphones. Of course, as would be readily understood, other connection types (such as those discussed throughout this disclosure) may be utilized. In this embodiment, it may be preferable for the device to be wirelessly connected to the headphones so that the device does not have to be located with the user. Additionally, as illustrated, the device includes an input for accepting a television audio signal, a computer readable medium (e.g., a memory card for storing the predetermined profile, filter coefficients and/or any noise that may be added to the audio signal), a processor and an output, each of which may be similar to those described with respect to FIG. 14.

In some embodiments, it may be important to distinguish between approved and non-approved signals. Specifically, depending on an individual's auditory system disorder, certain digital audio signals may not be amenable to spectral modification or may not be amenable to spectral modification beyond a certain extent. In these situations, the processor may be configured to distinguish between the two signals and therefore spectrally modify the signals to different extents. In some embodiments, the processor may not even spectrally modify a non-approved signal. For the processor to make such a determination, the processor may be configured to read the entire signal or may read a particular code recorded on the signal at some time prior to being used by the individual.

FIG. 16 is a functional schematic of a digital audio distribution system in accordance with certain embodiments. This embodiment focuses on the method for delivering digital audio signals to an individual or an individual's digital playback device. The digital playback device discussed with reference to these embodiments may be the same as the digital playback devices discussed herein or may be commercial off the shelf playback devices.

Generally, the system, in accordance with certain embodiments, utilizes a collection of digital audio tracks. One popular example of such a database is www.iTunes.com. This internet interface allows individuals to purchase digital audio tracks (or videos) individually and download them directly (or indirectly) to their digital playback device. Because this collection, as well as others, is stationary, it is possible for these databases to contain millions of audio tracks for an individual to select from.

In certain embodiments, a similar collection may be utilized or a new collection can be established. In either case, the individual may be able to access the system, and select at least one audio track. After selecting the audio track, the system can modify the digital audio track to create a spectrally modified digital audio track. The spectrally modified digital audio track is then provided to the user. In this manner, it may not be necessary for the digital playback device to include a processor for modifying the digital audio signal since it is modified before it is downloaded by the individual (although, as discussed above, it may be modified by the user as well). Additionally, although certain embodiments describe that the digital audio track is spectrally modified after it is selected, it also contemplates a collection of spectrally modified digital audio tracks even though such a collection may be less desirable in certain situations given the versatility and storage space that would be required to store all of the spectrally modified digital audio tracks.

In certain embodiments, the individual may interact with the system over the internet or in some other acceptable means such as by visiting a store front or ordering over the phone.

As discussed, there are several ways that exemplary systems can determine how or with which spectral modification signal to modify the digital audio signal. In certain embodiments, the system may request user information to determine which of the predetermined number of spectral modification signals to use to create the spectrally modified digital audio signal. Specifically, as discussed above in more detail, the system may have a number of “generic” spectral modification signals and may pick one depending on certain criteria and information obtained from the individual.

In certain embodiments (also detailed herein), the system may allow the user to provide to the system a spectral modification signal to modify the at least one digital audio track. In this embodiment, the individual may obtain the spectral modification signal from a clinic or similar entity.

In certain embodiments, the user may select one of a predetermined number of spectral modification signals to modify the digital audio track and, in some instances, the individual may obtain information from a clinic or similar entity or from a self administered test to determine which spectral modification signal to choose or the clinic or similar entity may indicate to the individual which signal to select. In a related embodiment, the clinic or similar entity may prescribe a certain spectral modification to an individual much like a drug prescription. Once the individual has the prescription, they will be able to provide the necessary information to the system such that the correct spectral modification signal is selected.

In yet another embodiment, the system may be able to gather enough information from an individual to create a customized or partially customized spectral modification signal. In this situation, the system may request information from the individual and may also administer certain tests to the individual, such as an auditory test or the like, and process the necessary data from the tests. In this manner, the individual may, in some embodiments, be able to obtain a customized spectral modification signal without visiting a specialized clinic, as described above with reference to exemplary embodiments.

As illustrated in the embodiment of FIG. 16, a physician interface is added so that the physician/clinician or entity creating the signals can directly interact with the system as well; in these embodiments, the physician/clinician or entity creating the signals may be able to provide spectral modifications to the system since the physician/clinician may be in the best position to determine how to produce generic signals. Alternatively, the physician/clinician or entity creating the signals could simply load the individual's specific spectral modification signal to the system and designate it as such. The user could then access the system and retrieve spectrally modified digital audio tracks based on their customized spectral modification signal. Additionally, by involving the physician/clinician or entity creating the signals, the system may also provide samples that could be provided to the individual.

Now that several embodiments of auditory system disorder devices and related methods have been described in detail, it will be apparent that the described devices for providing relief for persons suffering from an auditory system disorder, such as tinnitus and/or reduced sound tolerance, administered in combination with certain pharmaceutical agents, have a number of significant advantages over prior art techniques, including, but not limited to, the following:
i. by facilitating the use of a personal music player with relaxing music, it may be much more acceptable to patients than conventional devices, thereby promoting a pleasant listening experience, as well as compliance to treatment;
ii. by facilitating the use of a personal music player with relaxing music, it may facilitate relaxation, relief and a sense of control and hence ameliorates the limbic system reaction to tinnitus perception;
iii. by allowing the user to at least partially cover up their tinnitus perception, it may facilitate a sense of relief and control and hence ameliorates the limbic system reaction to tinnitus perception;
iv. by using a pharmaceutical agent which reduces neural activity, it may ameliorate the auditory system response to auditory deprivation or disruption involving increased or altered neural activity that may underlie tinnitus perception;
v. by using a pharmaceutical agent which modulates attentional focus, it may ameliorate the attentional focus on the tinnitus perception;
vi. by using a pharmaceutical agent which may facilitate a sense of relaxation or reduced state of arousal or anxiety or modulates the emotional response, it may ameliorate the limbic system reaction to tinnitus perception;
vii. by using a pharmaceutical agent which may facilitate neuroplastic change, it may enhance the effectiveness and/or efficiency of the use of acoustic stimulation to foster neuroplastic change within processes underlying an auditory system disorder;
viii. the algorithms developed to spectrally modify the audio stimuli correct for each individual's particular hearing loss configuration, as well as accounting for the effects of loudness recruitment, thus enabling effective stimulation at a relaxing intensity level, irrespective of each patient's audiometric profile;
ix. it compensates for high frequency hearing loss which accompanies tinnitus in approximately 80% of cases, thus providing the broadest spectrum of acoustic stimulation;
x. it compensates for each ear separately and delivers signals to each ear with control of the correlation between the two signals, thereby accounting for any asymmetry between the ears in order to facilitate stimulation at a comfortable listening level, as well as ensuring stimulation of the integrative pathways of the auditory system, and further enhancing the listening experience through provision of a stereo effect;
xi. intermittent tinnitus masking with music can provide a form of systematic desensitization to the disturbing effects of tinnitus; and,
xii. spectrally modified sound recordings produced using the algorithms reduced tinnitus distress to the point where it was no longer significantly interfering with quality of life in more than 75% of trial participants. Significant reductions in MMLs were measured, and levels of reduced sound tolerance had significantly improved.

It will also be apparent to persons skilled in the audiological and electronics arts that numerous variations and modifications may be made to the described auditory system disorder devices and related methods, in addition to those already described, without departing from the basic inventive concepts. For example, an algorithm may be employed to set the frequency response of existing tinnitus devices (e.g., maskers) which use bands of noise, rather than music, to achieve similar results. Various types of noise, pure tones and speech could also be used in addition to music. The same algorithms may also be employed in existing wireless receiver devices, such as the Starkey Silentia Set, or through hearing aid induction coil systems. Furthermore, the mathematical algorithms used for calculating the individual prescription of the audio signal may differ from the above-described algorithms, and extra sounds may also need to be inserted. However, other embodiments would be consistent with the clinical technique that is intended to provide a modification of the intensity of audio signals to account for hearing levels, specifically for the relief and/or treatment of auditory system disorders such as tinnitus and conditions of reduced sound tolerance (e.g. hyperacusis). All such variations and modifications are to be considered within the scope of the present inventions, the nature of which is to be determined from the foregoing description and the appended claims.

The pharmaceutical agents used in this disclosure may include one or more agents that lessen and/or alleviate primary and/or secondary sequelae or symptoms associated with an auditory system disorder (such as tinnitus), such as psychological, perceptual, attentional, and/or reactionary sequelae associated with the auditory system disorder. The pharmaceutical agent, in this regard, can be an agent that directly or indirectly lessens and/or alleviates symptoms associated with neuroplastic changes associated with, causing, and/or related to an auditory system disorder (such as tinnitus), such as neuroplastic changes in the auditory system that leads to an initial perception of a sound associated with an auditory system disorder by a auditory system disorder sufferer or subject, in attentional filters in the brain that cause the subject to pay attention to the auditory system disorder perception, and/or in regions of the limbic, emotional, and/or autonomic nervous system that cause an aversive reaction to auditory system disorder symptoms.

Suitable pharmaceutical agents may include, for example, anxiolytic agents, antidepressant agents, anticonvulsants, antiarrthymic agents, antihistamines, muscarinic agents, H₁ antagonists, opioid agents, osmotic regulators, vasodilators, anesthetic agents, NMDA (N-methyl-D-aspartic acid) receptor antagonists, diuretics, and/or other pharmaceutical agents or minerals that lessen and/or alleviate the primary and/or secondary symptoms associated with an auditory system disorder (such as tinnitus) or combinations thereof. In this regard, the pharmaceutical agents defined to be within any of these classes of pharmaceutical agents in Goodman & Gilman's The Pharmacological Basis of Therapeutics, 11^{th} Ed. (McGraw-Hill, 2007) can be utilized in the context of the present disclosure, in appropriate doses and dosage forms discussed therein.

Suitable anxiolytic agents may include, for example, benzodiazepines, azaspirones, beta-blockers, tricyclic amines (TMAs), monoamine oxidase inhibitors (MAOIs), serotonin reuptake inhibitors (SRIs), serotonin receptor agonists, and/or GABA receptor (e.g., GABA_{A} and GABA_{B} receptor) agonists and/or modulators (such as agents that modulate voltage-gated N-type calcium ion channels activity in GABA receptors). In certain embodiments, for example, the anxiolytic agent is a benzodiazepine, a non-benzodiazepine, and/or an agent having any antianxiety, sedative, and/or tranquilizing properties (such as a barbiturate or pharmacologically similar nonbarbiturate). In certain embodiments, the anxiolytic agent comprises one or more benzodiazepines, such as alprazolam (Xanax®) (8-chloro-1-methyl-6-phenyl-4H-s-triazolo[4,3-a][1,4]benzodiazepine), diazepam (Valium®), lorazepam (Ativan®), bromazepam, chlordiazepoxide, oxazepam, clorazepate, prazepam, midazolam, halazepam, estezolam, flurazepam, nitrazepam, quazepam, temazepam, triazolam, and/or clonazepam (Klonopin®) (which is also a potent anticonvulsant). In certain embodiments, the anxiolytic agent comprises one or more GABA receptor agonists and/or agents that promote GABA production, GABA release, and/or agents that lessen or slow GABA degradation, such as, for example, baclofen and/or gabapentin. In certain embodiments, the anxiolytic agent may comprise an azaspirone or azaspirodecanedione agent, such as buspirone, gepirone, ipsapirone, tandospirone, tiaspirone, or pharmaceutically acceptable salts thereof, or combinations or mixtures thereof. In certain embodiments, the anxiolytic agent may comprise gabapentin, sodium valproate, clozapine, a transtympanic steroid, an aminoglycosides, propanediol carbamate (such as meprobamate or tybamate), lidocaine, nortriptyline, a thyrotropin-releasing hormone, or pharmaceutically acceptable salts, esters, and/or derivatives thereof, or combinations or mixtures thereof. In certain embodiments, the anxiolytic agent comprises one or more non-benzodiazepine agents, such as imidazopyridines (such as zolpidem, alpiden, saridem, necopidem), pyrazolopyrimidines (such as zaleplon, indiplon, or ocinaplon), cyclopyrrolones (such as eszopiclone, zopiclone, pagoclone, suriclone, pazinaclone, or suproclone), or pharmaceutically acceptable salts, esters, and/or derivatives thereof, or combinations or mixtures thereof. In certain embodiments, the anxiolytic agent comprises a beta-receptor blocker, such as propranolol and/or oxprenolol, such as to combat the somatic symptoms of anxiety. In certain embodiments, the anxiolytic agent comprises an herbal extract having anxiolytic properties, such as, for example, ginkgo, valerian, kava, chamomile, kratom, blue lotus extracts, sceletium tortuosum, bacopa monniera, or pharmaceutically acceptable salts, esters, and/or derivatives thereof, or combinations or mixtures thereof.

Suitable antidepressant agents may include, for example, serotonin (5-HT) agonists (such as 5-HT_{IA}-receptor agonists), selective serotonin reuptake inhibitors (“SSRIs”), non-SRI agents, tricyclic antidepressant agents, norepinephrine-reuptake inhibitors (“NRIs”), monoamine-oxidase inhibitors (MAOIs), serotonin-and-noradrenaline-reuptake inhibitors (“SNFIs), corticotropin-releasing factor (CRF) antagonists, α-adrenoreceptor antagonists, NK1-receptor antagonists, and/or other antidepressants, or pharmaceutically acceptable salts, esters, and/or derivatives thereof, or combinations or mixtures thereof. Suitable antidepressant agents also include any suitable serotonin agonist, such as, for example, 2-methyl serotonin, buspirone, ipsaperone, tiaspirone, gepirone, ergot alkaloids, 8-hydroxy-(2-N,N-dipropyl-amino)-tetraline, 1-(4-bromo-2,5-dimethoxyphenyl)-2-aminopropane, cisapride, sumatriptan, m-chlorophenylpiperazine, trazodone, zacopride, mezacopride, or pharmaceutically acceptable salts, esters, and/or derivatives thereof, or a combinations or mixtures thereof. Suitable serotonin antagonists include, for example, ondansetron, granisetron, metoclopramide, tropisetron, dolasetron, palonosetron, trimethobenzamide, methysergide, risperidone, ketanserin, ritanserin, clozapine, amitriptyline, MDL 100,907 (R(+)-α-(2,3-dimethoxyphenyl)-1-[2-(4-fluorophenyl)ethyl]-4-piperidine-methanol) (Marion Merrell Dow), azatadine, cyproheptadine, fenclonine, chlorpromazine, mianserin, or pharmaceutically acceptable salts, esters, and/or derivatives thereof, or a combinations or mixtures thereof. Suitable antidepressant agents also include any suitable serotonin reuptake inhibitor, such as, for example, binedaline, m-chloropiperzine, citalopram, duloxetine (Cymbalta®), etoperidone, femoxetine, fluoxetine, fluvoxamine, indalpine, indeloxazine, milnacipran, nefazodone, oxaflazone, paroxetine, prolintane, ritanserin, sertraline (Zoloft®), tandospirone, venlafaxine (Effexor®), zimeldine, or a pharmaceutically acceptable salt, ester, or derivative thereof, or a combination or mixture thereof. Suitable antidepressant agents also include, for example, any suitable norepinephrine-reuptake inhibitor, such as, for example, amitriptyline, desmethylamitriptyline, clomipramine, doxepin, imipramine, imipramine-oxide, trimipramine; adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, or pharmaceutically acceptable salts, esters, and derivatives thereof, or combinations or mixtures thereof. Suitable antidepressant agents also include any suitable tricyclic antidepressant, such as any antidepressant agent having a tricyclic core, e.g., a iminodibenzyl, dibenzyocycloheptadiene, and/or dibenzyloxepinylidene core. Suitable tricyclic and tetracyclic antidepressants include, for example, amitryptiline, amoxapine, butriptyline, clomipramine, demexiptiline, desipramine, dibenzepin, dimetacrine, dothiepin, doxepin, imipramine, iprindole, lofepramine, maprotiline, melitracen, metapramine, mianserin, mirtazapine, nortryptiline, propizepine, protriptyline, quinupramine, setiptiline, tianeptine, trimipramine, or pharmaceutically acceptable salts, esters, and/or derivatives thereof, or a combinations or mixtures thereof. In some embodiments, the antidepressant agent comprises clomipramine (3-chloro-10,11-dihydro-N,N-dimethyl-5H-dibenz[b,f]azepine-5-propanamine), or a pharmaceutically acceptable salt, ester, or derivative thereof, such as a pharmacologically acceptable acid addition salts thereof, e.g., clomipramine hydrochloride. Suitable antidepressant agents also include any suitable monoamine oxidase inhibitor, such as, for example, amiflamine, brofaromine, clorgyline, α-ethyltryptamine, iproclozide, iproniazid, isocarboxazid, mebanazine, moclobemide, nialamide, pargyline, phenelzine, pheniprazine, pirlindole, safrazine, selegiline, toloxatone, tranylcypromine, or pharmaceutically acceptable salts, esters, and/or derivatives thereof, or a combinations or mixtures thereof. Suitable antidepressant agents also include any suitable azaspirone agent, such as, for example, buspirone, gepirone, ipsapirone, tandospirone, tiaspirone, or pharmaceutically acceptable salts, esters, or derivatives thereof, or combinations or mixtures thereof. Suitable antidepressant agents also include any suitable non-SRI antidepressant agent, such as, for example, amesergide, amineptine, benactyzine, bupropion, fezolamine, levoprotiline, medifoxamine, mianserin, minaprine, oxaflozane, oxitriptan, rolipram, teniloxazine, tofenacin, trazadone, tryptophan, viloxazine, or a pharmaceutically acceptable salt, ester, or derivative thereof, or a combination or mixture thereof.

Suitable anticonvulsant agents may include, for example, aldehydes, barbiturates, benzodiazepines, bromides, carboxamides, fructose derivatives, GABA receptor (e.g., GABA_{A} and/or GABA_{B} receptor) agonists and/or modulators (such as agents that modulate voltage gated N type calcium ion channels activity in GABA receptors), hydantoins, oxazolidinediones, proprionates, succinimide, triazines, caroverine, or pharmaceutically acceptable salts, esters, and/or derivatives thereof, or a combinations or mixtures thereof. In some embodiments, the anticonvulsant agent comprises of an aldehyde, such as paraldehyde (Paral®). In some embodiments, the anticonvulsant agent comprises one or more barbiturates such as phenobarbital (Luminal®), methylphenobarbital, barbexoclone. secobarbital, amobarbital, butalbital, cyclobarbital, pentobarbital, vinylbital, or pharmaceutically acceptable salts, esters, and/or derivatives thereof, or a combinations or mixtures thereof. In some embodiments, the anticonvulsant agent comprises one or more benzodiazepines, such as alprazolam (Xanax®) (8-chloro-1-methyl-6-phenyl-4H-s-triazolo[4,3-a][1,4]benzodiazepine), diazepam (Valium®), lorazepam (Ativan®), bromazepam, chlordiazepoxide, oxazepam, clorazepate, prazepam, midazolam, halazepam, estezolam, flurazepam, nitrazepam, quazepam, temazepam, triazolam, clonazepam (Klonopin®), or pharmaceutically acceptable salts, esters, and/or derivatives thereof, or a combinations or mixtures thereof. In some embodiments, the anticonvulsant agent comprises clonazepam. In some embodiments, the anticonvulsant agent comprises one or more bromides such as potassium bromide. In some embodiments, the anticonvulsant agent comprises one or more carboxamides, such as carbamazepine (Tegretol®), oxcarbazepine, rufinamide, or pharmaceutically acceptable salts, esters, and/or derivatives thereof, or a combinations or mixtures thereof. In some embodiments, the anticonvulsant agent comprises one or more fructose derivatives such as topiramate. In some embodiments, the anticonvulsant agent comprises one or more (i) GABA receptor agonists, (ii) agents that promote GABA production and/or GABA release, and/or (iii) agents that inhibit, decrease, lessen, and/or slow GABA degradation, such as, for example, pregabalin, gabapentin, or pharmaceutically acceptable salts, esters, and/or derivatives thereof, or a combinations or mixtures thereof. In some embodiments, the anticonvulsant agent may comprise gabapentin, sodium valproate, vigabatrin, progabide, tiagabine, or a pharmaceutically acceptable salt, ester, or derivative thereof, or a combination or mixture thereof. In some embodiments, the anticonvulsant agent comprises one or more hydantoins, such as ethotoin, fosphenytoin, mephenyloin, phenyloin, or pharmaceutically acceptable salts, esters, and/or derivatives thereof, or a combinations or mixtures thereof. In some embodiments, anticonvulsant agent comprises one or more oxazolidinediones such as ethadione, paramethadione, trimethadione, or a pharmaceutically acceptable salt, ester, or derivative thereof, or a combination or mixture thereof. In some embodiments, the anticonvulsant agent comprises one or more proprionates, such as beclamide. In some embodiments, the anticonvulsant agent comprises one or more succinimides, such as ethosuximide, mesuximide, phensuximide, or a pharmaceutically acceptable salt, ester, or derivative thereof, or a combination or mixture thereof. In some embodiments, the anticonvulsant agent comprises one or more triazines, such as lamotrigene.

Suitable antiarrhythmic agents may include, for example, beta blockers, sodium channel blockers, potassium channel blockers, calcium channel blockers, or combinations or mixtures thereof. In some embodiments, the anti arrhythmic agent comprises one or more beta blockers such as propranolol, sotalol, metoprolol, atenolol, or a pharmaceutically acceptable salt, ester, or derivative thereof, or a combination or mixture thereof. In some embodiments, the antiarrhythmic agent comprises one or more sodium channel blockers such as lidocaine, disopyramide, flecainide, procainamide, phenyloin, or a pharmaceutically acceptable salt, ester, or derivative thereof, or a combination or mixture thereof. In some embodiments, the antiarrhythmic agent comprises one or more calcium channel blockers such as verapamil, diltiazem, or a pharmaceutically acceptable salt, ester, or derivative thereof, or a combination or mixture thereof. In some embodiments, the anti arrhythmic agent comprises one or more potassium channel blockers, such as amiodarone, sotalol, or a pharmaceutically acceptable salt, ester, or derivative thereof, or a combination or mixture thereof.

Suitable muscarinic agents may include, for example, muscarinic receptor agonists, partial muscarinic receptor agonists, mixed muscarinic receptor agonists/antagonists, acetylcholinesterase inhibitors, or a combination or mixture thereof. In some embodiments, the muscarinic agent comprises pilocarpine, muscarine, donepezil, tacrine (THA), pyrodostigmine, physostigmine, huperzine, carbamates, thiaphysovenine, phenserine, endrophonium, demarcarium, ambenonium, scopolamine, or a pharmaceutically acceptable salt, ester, or derivative thereof, or a combination or mixture thereof. In some embodiments, the muscarinic agent comprises scopolamine.

Suitable H₁ antagonists may include, for example, ethylenediamines, ethanolamines, alkylamines, piperazines, trycyclics and tetracyclics, or a combination or mixture thereof. In some embodiments, the H₁ antagonist comprises one or more ethylenediamines such as mepyramine, antazoline, acrivisitine, or a pharmaceutically acceptable salt, ester, or derivative thereof, or a combination or mixture thereof. In some embodiments, the H₁ antagonist comprises an ethanolamine diphenhydramine, carbinoxamine, doxylamine, clemastine, dimenhydrinate, or a pharmaceutically acceptable salt, ester, or derivative thereof, or a combination or mixture thereof. In some embodiments, the H₁ antagonist comprises one or more alkylamines such as chlorphenamine, dexchlorpheniramine, brompheniramine, or a pharmaceutically acceptable salt, ester, or derivative thereof, or a combination or mixture thereof. In some embodiments, the H₁ antagonist comprises one or more piperazines such as promethazine, cinnarazine, or a pharmaceutically acceptable salt, ester, or derivative thereof, or a combination or mixture thereof.

Suitable opioids may include therapeutically useful and/or pharmacologically active opioids, opioid metabolites, and/or any enantiomers and/or diastereiomers thereof, including, but not limited to, anilopam, fentanyl, pentazocine, dihydroetorphine, phenazocine, sufentanil, codeine, alfentanil, meperidine, morphine, propoxyphene, morphine sulfate ester, tramadol, hydromorphone, buprenorphine, oxymorphone, levohanol, methadone, L-acetylmethadol, oxycodone, etorphine, hydrocodone, normethadone, remifentanil, noroxycodone, dihydrocodeine, norlevorphanol, ethylmorphine, nalbuphine, hydromorphine, or pharmaceutically acceptable salts, esters, or derivatives thereof, or combinations or mixtures thereof. Suitable anesthetic agents include, for example, mexiletine and tocainide.

Suitable NMDA receptor agents may include, for example, NMDA receptor agonists, partial agonists, and/or mixed agonists/antagonists, as well as NMDA receptor antagonists. Suitable such NMDA receptor agents include, for example, memantine and carverine, and combinations and mixtures thereof.

An effective amount of the pharmaceutical agent administered to a particular subject will be dependent on the particular pharmaceutical agent, the auditory system condition or disorder being treated (such as the particular type of tinnitus being treated), the severity of the auditory system condition or disorder, the subject's weight, the mode of administration, and/or other pertinent factors known to the prescribing physician and/or provider of therapy. Example dosages of sample pharmaceutical agents are set forth in Table 1.

TABLE 1
Doses

| Nonproprietary Name | Example Trade Name | Daily Dose, mg | |
|---|---|---|---|
| Alprazolam | Xanax ® | 0.5-4 | |
| Chlordiazepoxide | Librium ® | 10-100 (oral dosage form) | |
| | | 25-300 (parenteral) | |
| Clonazepam | Klonopin ® | 0.5-20 | |
| Clorazepate | Tranxene ® | 7.5-90 | |
| Diazepam | Valium ® | 2-40 | |
| Halazepam | Paxipam ® | 20-160 | |
| Lorazepam | Ativan ® | 1-10 | |
| Oxazepam | Serax ®; Zaxopam ® | 30-120 | |
| Prazepam | Centrax ® | 10-60 | |
| Buspirone | Buspar ® | 15-60 | |
| Baclofen | Lioresal ® | 15-80 | |
| Gabapentin | Neurontin ® | 300-3600, e.g., 600-2400 | |
| Sodium Valproate | Epilim ® | 100, 200, 500 | |
| Zolpidem | Stilnox ® | 5-10 | mg |
| Zaleplon | Sonata ® | 5-20 | mg |
| Eszopiclone | Lunesta ® | 1-3 | mg |
| Sumitriptan | Imigran ® | 50-100 | mg |
| Trazolan | Trazodone ® | 50-100 | mg |
| Ondansetron | Zofran ® | 4-8 | mg |
| Risperidone | Risperdal ® | 1-4 | |
| Amitriptyline | Endep ® | 10-100 | mg |
| Chlorpromazine | Largactil ® | 10-100 | mg |
| Paraldehyde | Paral ® | 5-30 | ml |
| Phenobarbital | Luminal ® | 30-320 | |
| Carbamezapine | Tegretol ® | 100-1600 | |
| Oxcarbazepine | Trileptal ® | 150-1200 | |
| Topiramate | Topamax ® | 25-1000 | |
| Pregabalin | Lyrica ® | 25-600 | |
| Vigabatrin | Sabril ® | 500-4000 | |
| Phenytoin | Dilantin ® | 30-300 | |
| Ethosuximide | Zarontin ® | 250-1600 | |
| Amineptine | Ebixa ® | 5-20 | mg |
| Clozapine | Clopine ® | 12.5-900 | mg |
| Gentamycin | Gentamicin ® | 3-5 | mg/kg |
| Meprobamate | Meprobamate ® | 1200-2400 | mg |
| Lidocaine | Lidocaine ® | 120 to 300 | mg |
| Nortriptyline | Aventyl Hydrochloride Pulvules ® | 10 to 150 | mg |
| Propranolol | Propranolol ® | 80-640 | mg |
| Duloxetine | Cymbalta ® | 20-60 | mg |
| Fluoxetine hydrochloride | Prozac ® | 20-80 | mg |
| Fluvoxamine Maleate | Fluvoxamine Maleate ® | 100-300 | mg |
| Sertraline | Zoloft ® | 100-200 | mg |
| Venlafaxine | Effixor ® | 75-375 | mg |
| Clomipramine | Clomipramine ® | 25-250 | mg |
| imipramine | Imipramine ® | | |
| mirtazapine | Mitrazipine ® | 15-45 | mg |
| Bupropion | Bupropion SR ® | 150-450 | mg |
| Phenelzine | Nardil ® | 15-60 | mg |
| Moclobemide | Amira ® | 150-600 | mg |
| Doxepin | Deptran ® | 150-450 | mg |
| Atenolol | Tenormin ® | 50-200 | |
| Metoprolol | Betaloc ® | 50-400 | |
| Sotalol | Sotalol ® | 80-640 | |
| Disopyramide | Rythmodan ® | 300-800 | |
| Verapamil | Isoptin ® | 80-320 | |
| Codeine | Paverel ® | 1-400 | |
| Donepezil | Aricept ® | 5-10 | |
| Hyoscine hydrobromide | Scopolamine ® | 0.01-0.1 | |
| Lamotrigene | Lamicitil ®, Elmendos ® | 2-200 | mg |
| Diphenhydramine | Benadryl ® | 25-50 | mg |
| | | 40 | ml |
| Theophylline | Theo24 ® | 190-1200 | mg |
| Ethylenediamine | | | |
| Carbinoxamine Maleate | Histex CT ® (Tablet)/Histex IE ® | 12-64 | mg |
| | (Capsule)/Histex PD ® (liquid) | | |
| Acrivastine | Semprex D ® | 32-48 | mg |
| Antazoline sulfate | Antistine-Privine ® Eye Drops | 7.5-20 | mg |
| Doxylamine | UniSom ® | 25-50 | mg |
| Clemastine | Clemastine fumarate ® | 1.34-8.04 | mg/day |
| Dimenhydrinate | Calm-X ® | 50-400 | mg/day |
| Chlorpheniramine | Aller-Chlor ® | 8-32 | mg/day |
| Dexchlorpheniramine | Dexchlorpheniramine Maleate ® | 0.5-8 | mg/day |
| Brompheniramine | Bidhist ® | 12-48 | mg |
| Promethazine | Promethazine VC ® w/Codeine | 6.25-75 | mg |
| Piperazine Estrone | Ogen ® | 0.625 to 5 | mg |
| Sulfate | | | |
| mexiletine | Mexiletine hydrochloride | 600-1200 | mg |
| tocainide | Tonocard ® | 1200-2400 | mg |
| memantine | Namenda ® | 5-20 | mg |
| dexamethasone | Dexamethasone ® | 0.75 mg to 30 mg | |
| prednisone | Prednisone ® | 5-60 | mg |
| frusemide | Frusemide ® | 20-600 | mg |
| misoprostol | Misoprostol ® | 200-800 | mcg |
| Caroverine | | Single infusions up to | |
| | | 160 mg | |
| Mannitol | | 1-30%, such as 10- | |
| | | 25%, such as 18% iv | |
| Glycerol | | 1-20%, such as 5-15%, | |
| | | such as 10% iv | |
| Midazolam | Midazolam Hydrchloride ® | 5 mg/5 ml-10 mg/50 ml | |
| Acamprosate | Campral ® | 1332-1998 | mg |
| Rivastigmine | Exelon ® | 1.5-6.0 | mg |
| Galantamine | Razadyne ® | 8-24 | mg |
| Dextroamphetamine | Dexedrine ® | 2.5-60 | mg |
| Methylphenidate | Ritalin ® | 5-60. | mg |

The dosage to be administered of the pharmaceutical agents or combinations or mixtures thereof disclosed can be calculated using a number of suitable manners, such as on the basis of the weight of agent per kg by weight of subject per day. Other means of calculating acceptable dosages are readily available to those skilled in the art.

An effective amount of an anxiolytic agent may vary among anxiolytic agents. For example, an effective amount of alprazolam may be about 0.25-4 mg/day, 0.25-3 mg/day, 0.5-1.5 mg/day, 0.5-1.25 mg/day, 0.75-1.25 mg/day, or 0.5-1.0 mg/day. In certain embodiments, the amount is about 1 mg/day. In certain embodiments, the amount is about 0.25-0.5 mg/day. The dose may be increased to achieve a maximum therapeutic effect, at an interval of two weeks, to a maximum daily dose of 1 mg, given in divided doses. In certain embodiments, 0.5 mg of alprazolam is administered to the subject three times daily.

An effective amount of lorazepam may be about 1-10 mg/day, 1-2.5 mg/day, or 2.5-3 mg/day. In certain embodiments, the amount is about 1 mg/day. In certain embodiments, the amount is about 2-3 mg/day. The dose may be increased to achieve a maximum therapeutic effect, at an interval of two weeks, to a maximum daily dose of 10 mg, given in divided doses. In certain embodiments, 1 mg of lorazepam is administered to the subject three times daily.

An effective amount of an antidepressant agent may vary among antidepressant agents. For example, an effective amount of buspirone may be about 0.25-50 mg/day, 0.5-40 mg/day, 0.75-30 mg/day, or 1.0-20 mg/day.

An effective amount per day of trazadone may vary, for example it may be about 25-600 mg/day, 50-500 mg/day, 100-450 mg/day, 150-350 mg/day, 200-400 mg/day, or 250-300 mg/day. In certain embodiments, the dose is about 150 mg/day. The dose may be increased to achieve a maximum therapeutic effect, at an interval of two weeks, to a maximum daily dose of 600 mg, given in divided doses.

An effective amount per day of ondansetron may vary, for example, it may be about 1-40 mg/day, 2-30 mg/day, 4-24 mg/day, or 8-16 mg/day. In certain embodiments, the amount is about 16 mg/day. The dose may be increased to achieve a maximum therapeutic effect to a maximum daily dose of 24 mg, given in divided doses.

An effective amount per day of risperidone may vary, for example, it may be about 0.25-10 mg, 0.5-8.0 mg, or 1.0-4.0 mg. In certain embodiments, the amount is about 2.0-3.0 mg/day. The dosage to be administered of risperidone can be calculated using a number of suitable manners, such as on the basis of from about 0.01 to 0.4 mg/kg by weight of subject per day.

An effective amount per day of amitriptyline, may vary. For example, it may be about 50-300 mg/day, 75-250 mg/day, or 100-200 mg/day. In certain embodiments, the amount is about 150-300 mg/day.

An effective amount per day of chlorpromazine, may vary. For example, it may be about 50-300 mg/day, 75-250 mg/day, or 100-200 mg/day. In certain embodiments, the amount is about 150-300 mg/day. The dose may be increased to achieve a maximum therapeutic effect to a maximum daily dose of 300 mg, given in divided doses.

An effective amount per day of citalopram may vary, for example, it may be about 1-100 mg/day, 10-80 mg/day, 20-60 mg/day, 20-40 mg/day, or 40-60 mg/day. In certain embodiments, the amount is about 20 to 60 mg. The dose may be increased to achieve a maximum therapeutic effect, to a maximum daily dose of 60 mg, given in divided doses.

An effective amount per day of duloxetine may vary. For example, it may be about 1-100 mg/day, 10-80 mg/day, 20-60 mg/day, 20-40 mg/day, or 40-60 mg/day. The dose may be increased to achieve a maximum therapeutic effect, to a maximum daily dose of 60 mg.

An effective amount per day of fluoxetine may vary, for example, it may be about 10-100 mg/day, 15-90 mg/day, 20-80 mg/day, 30-70 mg/day, or 40-60 mg/day. In certain embodiments, the amount of about 50-80 mg is administered to the subject daily.

An effective amount per day of fluvoxamine may vary, for example, it may be about 50-300 mg/day, 75-275 mg/day, 150-250 mg/day, or 200-300 mg/day. In certain embodiments, the amount is about 30-100 mg/day. The dose may be increased to achieve a maximum therapeutic effect, to a maximum daily dose of 300 mg, given in divided doses. In certain embodiments, 100 to 300 mg of fluvoxamine is administered to the subject three times daily.

An effective amount per day of sertraline may vary. For example, it may be about 50-200 mg/day, 75-175 mg/day, 100-150 mg/day, or 150-200 mg/day. In certain embodiments, the amount is about 100-200 mg/day. The dose may be increased to achieve a maximum therapeutic effect, to a maximum daily dose of 200 mg/day, given in divided doses. In certain embodiments, 100 to 200 mg of sertraline is administered to the subject three times daily.

An effective amount per day of venlafaxine may vary, for example, it may be about 75-375 mg/day, 100-350 mg/day, 150-300 mg/day, or 200-250 mg/day. In certain embodiments, the amount is about 150-225 mg/day. In certain embodiments, the amount is about 350 to 375 mg/day in divided doses. The dose may be increased to achieve a maximum therapeutic effect, to a maximum daily dose of 375 mg, given in divided doses.

An effective amount per day of clomipramine may vary, for example, it may be about 25-250 mg/day, 50-200 mg/day, or 100-175 mg/day. In certain embodiments, the amount is about 150-250 mg/day. The dose may be increased to achieve a maximum therapeutic effect, to a maximum daily dose of 250 mg/day, given in divided doses.

An effective amount per day of mirtazapine may vary, for example, it may be about 7.5-45 mg/day, 15-37.5 mg/day, 22.5-30 mg/day, or 15-37.5 mg/day. In certain embodiments, the amount is about 15-30 mg/day. The dose may be increased to achieve a maximum therapeutic effect, to a maximum daily dose of 45 mg

An effective amount per day of amineptine may vary, for example, it may be about 5-20 mg/day, 10-15 mg/day, or 15-20 mg/day. In certain embodiments, the amount is about 5-10 mg/day in 3 divided doses. The dose may be increased to achieve a maximum therapeutic effect to a maximum daily dose of 20 mg, given in divided doses.

An effective amount per day of bupropion may vary, for example, it may be about 100-500 mg/day, 150-450 mg/day, or 225-400 mg/day. In certain embodiments, the amount is about 150-300 mg/day. The dose may be increased to achieve a maximum therapeutic effect, to a maximum daily dose of 450 mg.

An effective amount per day of phenelzine may vary. For example, it may be about 45-90 mg/day, or 45-60 mg/day. In certain embodiments, the amount is about 15-45 mg/day. The dose may be increased to achieve a maximum therapeutic effect, to a maximum daily dose of 90 mg.

An effective amount per day of moclobemide may vary. For example, it may about 150-600 mg/day, 225-525 mg/day, 300-450 mg/day, or 375-600 mg/day. In certain embodiments, the amount is about 150-600 mg/day. The dose may be increased to achieve a maximum therapeutic effect, to a maximum daily dose of 600 mg.

An effective amount per day of doxepin may vary, for example, it may be about 30-450 mg/day, 45-425, or 60-400 mg/day. The dose may be increased to achieve a maximum therapeutic effect, to a maximum daily dose of 450 mg/day in three divided doses.

An effective amount per day of sumatriptan may vary. For example, it may be about 25-200 mg/day, 50-175 mg/day, 75-150 mg/day, or 100-125 mg/day. In certain embodiments, the amount is about 100 mg/day. The dose may be increased to achieve a maximum therapeutic effect, at an interval of two weeks, to a maximum daily dose of 200 mg, given in divided doses.

An effective amount per day of baclofen may vary, for example, it may be about 0.25-100 mg/day, 0.5-80 mg/day, 1-60 mg/day, 2-50 mg/day, or 4-40 mg/day. In certain embodiments, the amount is about 5 mg/day. In certain embodiments, the amount is about 5-15 mg/day. The dose may be increased to achieve a maximum therapeutic effect, at an interval of two weeks, to a maximum daily dose of 80 mg/day, given in divided doses. In certain embodiments, 15 mg of baclofen is administered to the subject three times daily.

An effective amount per day of gabapentin may vary, for example, it may be about 300-3600 mg/day, 600-2400 mg/day, or 900-1800 mg/day. In certain embodiments, the amount is about 1200 mg/day and in others the amount is about 1200-2400 mg/day. The dose may be increased to achieve a maximum therapeutic effect, at an interval of two weeks, to a maximum daily dose of 2400 mg, given in divided doses. In certain embodiments, 600 mg of gabapentin is administered to the subject three times daily.

An effective amount per day of sodium valproate may vary, for example, it may be about 400-2000 mg/day, 500-1500 mg/day, or 800-1000 mg/day. In certain embodiments, the amount is about 1000 mg/day. The dose may be increased to achieve a maximum therapeutic effect, at an interval of two weeks, to a maximum daily dose of 2000 mg, given in divided doses.

An effective amount per day of zolpidem may vary, for example, it may be about 2.5-10 mg/day, 2.5-7.5 mg/day, 5.0-10 mg/day, or 7.5-10 mg/day. In certain embodiments, the amount is about 10 mg/day.

An effective amount per day of zaleplon may vary, for example, it may be about 2.5-20 mg/day, 5-17.5 mg/day, 7.5-15 mg/day, or 10-12.5 mg/day. In certain embodiments, the amount is about 10 mg/day.

An effective amount per day of eszopiclone may vary, for example, it may be about 0.5-3 mg/day, 1-2.5 mg/day, 1.5-2 mg, or 1.0-2.5 mg/day. In certain embodiments, the amount is about 2 mg/day.

An effective amount per day of paraldehyde may vary, for example, it may be about 5-30 ml/day, 10-25 ml/day, or 15-20 ml/day. In certain embodiments, the amount is about 15 ml/day. The dose may be increased to achieve a maximum therapeutic effect to a maximum daily dose of 30 ml, given in divided doses.

An effective amount per day of phenobarbital may vary, for example, it may be about 30-320 mg/day, 60-250 mg/day, or 90-120 mg/day. In certain embodiments, the amount is 10-15 mg/kg. The dose may be increased to achieve a maximum therapeutic effect to a maximum daily dose of 320 mg, given in divided doses.

An effective amount of an anticonvulsant agent may vary, for example, with clonazepam it may be about 0.25-2 mg/day, 0.5-1.5 mg/day, 0.5-1.25 mg/day, or 0.75-1.25 mg/day. In certain embodiments, the amount is about 1 mg/day. In certain embodiments, the amount is about 0.25-0.5 mg/day. The dose may be increased to achieve a maximum therapeutic effect, at an interval of two weeks, to a maximum daily dose of 1 mg, given in divided doses. In certain embodiments, 0.5 mg of clonazepam is administered to the subject three times daily.

An effective amount per day of oxcarbazepine may vary, for example, it may be about 150-1200 mg/day, 300-1050 mg/day, 450-900 mg/day, or 600-750 mg/day. In certain embodiments, the amount is about 300-900 mg/day. In certain embodiments, the amount is 600 mg/day. The dose may be increased to achieve a maximum therapeutic effect to a maximum oxcarbazepine daily dose of 1200 mg, given in divided doses.

An effective amount per day of carbamezapine may vary, for example, it may be about 100-1600 mg/day, 200-1500 mg/day, 300-1400 mg/day, 400-1000 mg/day, or 500-700 mg/day. In certain embodiments, the amount is about 200-800 mg/day. In certain embodiments, the amount is 400 mg/day. The dose may be increased to achieve a maximum therapeutic effect to a maximum daily dose of 1600 mg, given in divided doses.

An effective amount per day of topiramate may vary, for example, it may be about 25-1000 mg/day. In certain embodiments, the amount is about 50-500 mg/day. In certain embodiments, the amount is 200 mg/day. The dose may be increased to achieve a maximum therapeutic effect to a maximum daily dose of 1000 mg, given in divided doses.

An effective amount per day of pregabalin may vary, for example, it may be about 25-600 mg/day, 50-300 mg/day, or 75-100 mg/day. In certain embodiments, the amount is about 200 mg/day. The dose may be increased to achieve a maximum therapeutic effect, at an interval of two weeks, to a maximum daily dose of 600 mg, given in divided doses. In certain embodiments, 200 mg of pregabalin is administered to the subject three times daily.

An effective amount per day of vigabatrin may vary, for example, it may be about 500-4000 mg/day, 1000-3000 mg/day, or 2000 mg/day. The dose may be increased to achieve a maximum therapeutic effect, at an interval of two weeks, to a maximum daily dose of 4000 mg, given in divided doses.

An effective amount per day of phenyloin may vary, for example, it may be about 30-300 mg/day, 60-200 mg/day, 90-290 mg/day, 100-260 mg/day, or 120-180 mg/day. The dose may be increased to achieve a maximum therapeutic effect, at an interval of two weeks, to a maximum daily dose of 300 mg, given in divided doses.

An effective amount per day of ethosuximide may vary, for example, it may be about 250-1500 mg/day, 500-1250 mg/day, 750-1000 mg/day, or 125-875 mg/day. The dose may be increased to achieve a maximum therapeutic effect, at an interval of two weeks, to a maximum daily dose of 30 mg/kg, given in divided doses.

An effective amount per day of lamotrigine may vary, for example, it may be about 5-200 mg/day, 25-100 mg/day, 50-150 mg/day, or 75-125 mg/day. The dose may be increased to achieve a maximum therapeutic effect, at an interval of two weeks, to a maximum daily dose of 5 mg/kg, given in divided doses.

An effective amount of an antiarrhythmic agent may vary. For example, an effective amount per day of propranolol may vary. In certain embodiments, an effective amount of propranolol may be about 80-640 mg/day, 120-160 mg/day, 160-240 mg/day, or 320 mg/day. In certain embodiments, the amount is about 80-320 mg/day. In certain embodiments, the amount is about 120-320 mg/day in divided doses. The dose may be increased to achieve a maximum therapeutic effect, to a maximum daily dose of 640 mg, given in divided doses. In certain embodiments, 80-320 mg of propranolol is administered to the subject three times daily. An effective amount of metoprolol may be about 50-400 mg/day, 100-2000 mg/day, 150-250 mg/day, or 200 mg/day. The dose may be increased to achieve a maximum therapeutic effect, to a maximum daily dose of 400 mg, given in divided doses. An effective amount of atenolol may be about 50-200 mg/day, or 100-150 mg/day. In certain embodiments, the amount is about 100 mg/day. The dose may be increased to achieve a maximum therapeutic effect, to a maximum daily dose of 200 mg, given in divided doses.

An effective amount per day of sotalol may vary, for example, it may be about 80-640 mg/day, 120-160 mg/day, 160-240 mg/day, or 320 mg/day. In certain embodiments, the amount is about 80-320 mg/day. In certain embodiments, the amount is about 120-360 mg/day in divided doses. The dose may be increased to achieve a maximum therapeutic effect, to a maximum daily dose of 640 mg, given in divided doses.

An effective amount per day of lidocaine may vary, for example, it may be about 120-300 mg/day, 140-280 mg, 180-200 mg/day, or 200-300 mg/day. In certain embodiments, the amount is below 4.5 mg/kg (2 mg/lb) body weight. The dose may be increased to achieve a maximum effect, to a maximum daily dose of 300 mg.

An effective amount per day of disopyramide may vary, for example, it may be about 300-800 mg/day, 400-700 mg/day, 450-650 mg/day, or 500-600 mg/day. The dose may be increased to achieve a maximum effect, to a maximum daily dose of 800 mg.

An effective amount per day of verapamil may vary, for example, it may be about 80-320 mg/day, 120-240 mg/day, or 160-200 mg/day. In certain embodiments, the amount is about 160-240 mg/day in divided doses. The dose may be increased to achieve a maximum therapeutic effect, to a maximum daily dose of 320 mg, given in divided doses.

An effective amount of an opioid agent may vary among each opioid agent. For example, an effective amount per day of pharmaceutical formulations comprising codeine may vary. In certain embodiments, an effective amount of codeine may be about 1-400 mg/day, 5-360 mg/day, or 10-100 mg/day. In certain embodiments, the amount is about 120 mg/day. In certain embodiments, 30 mg of codeine is administered to the subject three times daily.

An effective amount of tramadol may be about 1-400 mg/day, 5-360 mg/day, or 10-100 mg/day. In certain embodiments, the amount is about 100 mg/day. In certain embodiments, 50 mg of codeine is administered to the subject four times daily.

An effective amount of a muscarinic agent may vary among each muscarinic agent. For example, an effective amount per day of donepezil may vary. In certain embodiments, an effective amount of donepezil may be about 5-10 mg/day. In certain embodiments, the amount is about 5 mg/day. In certain embodiments, 10 mg of donepezil is administered to the subject once daily.

An effective amount of scopolamine may be about 1-100 mg/day, 5-90 mg/day, or 10-80 mg/day (such as, 9.9-79.2 mg/day). In certain embodiments, the amount is about 20-60 mg/day (such as about 19.8-58.4 mg/day). In certain embodiments, 19.8 mg of scopolamine is administered to the subject four times daily.

An effective amount per day of ethylenediamine may vary, for example, it may be about 190-1200 mg/day, 240-1000 mg/day, 300-900 mg/day, or 400-800 mg/day. In certain embodiments, the amount is below 0.1-3.1 mg/kg body weight/hr. The dose may be increased to achieve a maximum effect, to a maximum daily dose of 1200 mg.

An effective amount per day of ethanolamine may vary, for example, it may be about 12-64 mg/day, 14-60 mg/day, 18-56 mg/day, or 22-52 mg/day. In certain embodiments, the amount is about 30-50 mg/day. In certain embodiments, the amount is about 40-60 mg/day in divided doses. The dose may be increased to achieve a maximum therapeutic effect, to a maximum daily dose of 64 mg, given in divided doses.

An effective amount per day of acrivastine may vary, for example, it may be about 10-400 mg/day, 24 mg-322 mg/day, 32-300 mg/day, 40-280 mg, 48-240 mg/day, or 56-220 mg/day. In certain embodiments, the amount is about 32 to 48 mg. The dose may be increased to achieve a maximum therapeutic effect, to a maximum daily dose of 322 mg, given in divided doses.

An effective amount per day of antazoline may vary, for example, it may be about 7.5-20 mg/day, 9.5-18.5 mg/day, 10-18 mg/day, or 10.5-17.5 mg/day. In certain embodiments, the amount is about 12-16 mg/day. In certain embodiments, the amount is about 7.5-20 mg/day in divided doses. The dose may be increased to achieve a maximum therapeutic effect, to a maximum daily dose of 40 mg, given in divided doses.

An effective amount per day of diphenhydramine may vary, for example, it may be about 10-400 mg/day, 50-350 mg, 75-325 mg/day, or 125-275 mg/day. In certain embodiments, the amount is about 150-250 mg/day. The dose may be increased to achieve a maximum therapeutic effect, to a maximum daily dose of 400 mg, given in divided doses.

An effective amount per day of doxylamine may vary, for example, it may be about 25-100 mg/day, 37.5-75 mg/day, or 50-62.5 mg/day. The dose may be increased to achieve a maximum therapeutic effect, to a maximum daily dose of 100 mg, given in divided doses.

An effective amount per day of clemastine may vary, for example, it may be about 1.34 mg-8.04 mg/day, 2.68-6.70 mg, or about 5.36-6.34 mg/day. In certain embodiments, the amount is about 2.68 to 8.04 mg/day. The dose may be increased to achieve a maximum therapeutic effect, to a maximum daily dose of 8.04 mg, given in divided doses.

An effective amount per day of dimenhydrinate may vary, for example, it may be about 50-400 mg/day, 100-350 mg/day, 150-300 mg/day, or 200-250 mg/day. In certain embodiments, the amount is about 200-300 mg/day. The dose may be increased to achieve a maximum therapeutic effect, to a maximum daily dose of 400 mg, given in divided doses.

An effective amount per day of chlorpheniramine may vary, for example, it may be about 8-32 mg/day, 12-20 mg/day, or 16-20 mg/day. In certain embodiments, the amount is about 16-24 mg/day in divided doses. The dose may be increased to achieve a maximum therapeutic effect, to a maximum daily dose of 32 mg, given in divided doses.

An effective amount per day of dexchlorpheniramine may vary, for example, it may be about 0.5 mg-8 mg/day, 1.0-7.5 mg/day, or 1.5-7.0 mg/day. In certain embodiments, the amount is about 2.0-6.5 mg/day. In certain embodiments, the amount is about 3-6 mg/day. The dose may be increased to achieve a maximum therapeutic effect, to a maximum daily dose of 8 mg, given in divided doses.

An effective amount per day of brompheniramine may vary, for example, it may be about 12-48 mg/day, 20-40 mg/day, or 28-32 mg/day. In certain embodiments, the amount is about 12-24 mg/day. In certain embodiments, the amount is about 30-48 mg/day. The dose may be increased to achieve a maximum therapeutic effect, to a maximum daily dose of 48 mg, given in divided doses.

An effective amount per day of promethazine may vary, for example, it may be about 6.25-75 mg/day, 12.5-50 mg/day, or 25-50 mg/day. In certain embodiments, the amount is about 25-75 mg/day. In certain embodiments, the amount is about 6.25-12.5 mg/day. The dose may be increased to achieve a maximum therapeutic effect, to a maximum daily dose of 75 mg, given in divided doses.

An effective amount per day of piperazine may vary, for example, it may be about 0.625-5.0 mg/day, 1.2-4.4 mg/day, 1.8-4.0 mg/day, or 2.2-3.6 mg/day. In certain embodiments, the amount is about 2.5-5 mg/day. In certain embodiments, the amount is about 0.625-1.25 mg/day in divided doses. The dose may be increased to achieve a maximum therapeutic effect, to a maximum daily dose of 5 mg/day, given in divided doses.

An effective amount per day of mexiletine may vary, for example, it may be about 400-1600 mg/day, 600-1200 mg/day, 700-1150 mg/day, or 750-1000 mg/day. In certain embodiments, the amount is about 850-950 mg/day. In certain embodiments, the amount is about 400-800 mg/day. The dose may be increased to achieve a maximum therapeutic effect, to a maximum daily dose of 1200 mg, given in divided doses.

An effective amount per day of tocainide may vary, for example, it may be about 800-3000 mg/day, 1200-2400 mg/day, 1400-2200 mg/day, or 1600-2000 mg/day. In certain embodiments, the amount is about 1800-2400 mg/day. In certain embodiments, the amount is about 1200-1800 mg/day. The dose may be increased to achieve a maximum therapeutic effect, to a maximum daily dose of 2400 mg, given in divided doses.

An effective amount per day of memantine may vary, for example, it may be about 0.5-50 mg/day, 1-40 mg/day, 3-30 mg/day, 5-20 mg/day, 10-20 mg/day, or 15-20 mg/day. In certain embodiments, the amount is about 5-15 mg/day. In certain embodiments, the amount is about 5-10 mg/day. The dose may be increased to achieve a maximum therapeutic effect, to a maximum daily dose of 20 mg, given in divided doses.

An effective amount per day of dexamethasone may vary, for example, it may be about 0.5-40 mg/day, 0.75-30 mg/day, 2.5-24 mg/day, or 3.25-18 mg/day. In certain embodiments, the amount is about 4-12 mg/day. In certain embodiments, the amount is about 5.5-6 mg/day. The dose may be increased to achieve a maximum therapeutic effect, to a maximum daily dose of 30 mg, given in divided doses.

An effective amount per day of prednisone may vary, for example, it may be about 0.5-100 mg/day, 1-80 mg/day, 5-60 mg/day, 25-40 mg/day, or 15-30 mg/day. In certain embodiments, the amount is about 20-40 mg/day. In certain embodiments, the amount is about 5-10 mg/day. The dose may be increased to achieve a maximum therapeutic effect, to a maximum daily dose of 60 mg, given in divided doses.

An effective amount per day of frusemide may vary, for example, it may be about 1-800 mg/day, 10-600 mg/day, 20-500 mg/day, 40-400 mg/day. In certain embodiments, the amount is about 80-200 mg/day. In certain embodiments, the amount is about 100-140 mg/day. The dose may be increased to achieve a maximum therapeutic effect, to a maximum daily dose of 600 mg, given in divided doses.

An effective amount per day of misoprostol may vary, for example, it may be about 200-1000 mcg/day, 400-800 mcg/day, 500-700 mcg/day, or 600-650 mcg/day. In certain embodiments, the amount is about 450-550 mcg/day. In certain embodiments, the amount is about 500-800 mcg/day. The dose may be increased to achieve a maximum therapeutic effect, to a maximum daily dose of 800 mcg, given in divided doses.

An effective amount per day of rivastigmine may vary, for example, it may be about 1.5-6.0 mg/day, 3.0-4.5 mg/day, or 4.5-6.0 mg/day. The dose may be increased to achieve a maximum therapeutic effect, to a maximum daily dose of 6 mg, given in divided doses.

An effective amount per day of galantamine may vary, for example, it may be about 8-24 mg/day, 8-16 mg/day, or 16-24 mg/day. The dose may be increased to achieve a maximum therapeutic effect, to a maximum daily dose of 24 mg, given in divided doses An effective amount per day of dextroamphetamine may vary, for example, it may be about 2.5-60 mg/day, 5-50 mg/day, or 10-40 mg/day. The dose may be increased to achieve a maximum therapeutic effect, to a maximum daily dose of 60 mg, given in divided doses

An effective amount per day of methylphenidate may vary, for example, it may be about 5-60 mg/day, 10-50 mg/day, or 15-40 mg/day. The dose may be increased to achieve a maximum therapeutic effect, to a maximum daily dose of 60 mg, given in divided doses

An effective amount per day of acamprosate may vary, for example, it may be about 333-1998 mg/day, 666-1665 mg/day, or 999-1332 mg/day. The dose may be increased to achieve a maximum therapeutic effect, to a maximum daily dose of 1998 mg, given in divided doses

An effective amount per day of midazolam may vary, for example, it may be about 1 mg/1 ml-5 mg/ml. The dose may be increased to achieve a maximum therapeutic effect, to a maximum daily dose of 5 mg, given in divided.

Any of the discussed pharmaceutical agents may be administered in the form of a pharmaceutically acceptable salt, ester, amide, prodrug, active metabolite, conjugate, derivative, or the like, provided that the salt, ester, amide, prodrug, metabolite, conjugate or other derivative is suitable pharmacologically, i.e., effective. Salts, esters, amides, prodrugs, conjugates and other derivatives of the active agents may be prepared using procedures available to those skilled in the art. In addition, any pharmaceutical agent that comprises chiral centers can be in the form of a single isomer or a racemic mixture of isomers. Chiral pharmaceutical agents may be in isomerically pure form, or they may be administered as a racemic mixture of isomers. Other derivatives and analogs of the pharmaceutical agents may be prepared using techniques available to those skilled in the art.

The pharmaceutical agents can be administered by various routes that are compatible with the desired outcome. Thus, the pharmaceutical agents may be administered orally (e.g., ingestion or inhalation), intraperitoneally, intradermally, transdermally, transmucosally, subcutaneously, sublingually, intravenously, intraarterially, intracavity, intracranially, intratympanically, intramuscularly, parenterally, topically, or combinations thereof. The pharmaceutical agents can be administered alone or in combination with pharmaceutically acceptable carriers, excipients, or diluents, and such administration may be carried out in single or multiple doses. More particularly, the pharmaceutical agents can be administered in a wide variety of different dosage forms, i.e., they may be combined with various pharmaceutically acceptable inert carriers or excipients in the form of tablets, capsules, emulsions, lozenges, troches, hard candies, lollipops, powders, sprays, creams, salves, suppositories, jellies, gels, pastes, lotions, ointments, aqueous suspensions, injectable solutions, elixirs, syrups, injectable depots, implants, microencapsulated delivery systems, oil-based suspensions, and the like. A solid unit dosage form can be prepared in any suitable manner. Oral dosage forms in certain embodiments may be preferred for certain pharmaceutical agents, and include tablets, capsules, caplets, solutions, suspensions and/or syrups, and may also comprise a plurality of granules, beads, powders or pellets that may or may not be encapsulated. Such dosage forms are prepared using methods available to those in the field of pharmaceutical formulation. For preparing solid compositions such as tablets, the active ingredient can be mixed with conventional ingredients such as talc, magnesium stearate, dicalcium phosphate, magnesium aluminum silicate, calcium sulfate, starch, lactose, acacia, methylcellulose, and functionally similar materials as pharmaceutical carriers. Wafers can be prepared in the same manner as tablets, differing only in shape and the inclusion of sucrose or other sweetener and flavor. In certain embodiments, capsules, like tablets, are prepared by mixing the compound with an inert pharmaceutical diluent and filling the mixture into a hard gelatin capsule of appropriate size.

Fluid unit dosage forms for oral administration, such as syrups and suspensions, can be prepared. The water-soluble forms can be dissolved in an aqueous vehicle together with sugar, aromatic flavoring agents and preservatives to form syrup. Suspensions can be prepared with a syrup vehicle with the aid of a suspending agent such as acacia, methylcellulose and the like.

For parenteral administration, fluid unit dosage forms can be prepared utilizing the compound and a sterile vehicle, such as water. The compound, depending on the vehicle and concentration used, can be either suspended or dissolved in the vehicle. In preparing solutions, the compound can be dissolved in water for injection and filter sterilized before filling into a suitable vial or ampoule and sealing. Parenteral suspensions can be prepared in the same manner, except that the compound is suspended in the vehicle instead of being dissolved and sterilization cannot be accomplished by filtration. The compound can be sterilized by exposure to ethylene oxide before suspending in the sterile vehicle.

In certain embodiments, alprazolam may be taken orally. In certain embodiments, alprazolam is in the form of an extended release oral dosage form. In certain embodiments, alprazolam is administered in the form of an immediate release tablet (available, for example, as Xanax® tablets from PHARMACIA AND UPJOHN) in the dose strengths of 0.25 mg, 0.5 mg, 1 mg, or 2 mg. In certain embodiments, alprazolam is administered in the form of an extended release tablet (available, for example, as Xanax® XR tablets from PHARMACIA AND UPJOHN) in dose strengths of 0.5 mg, 1 mg, 2 mg, or 3 mg.

In certain embodiments, lidocaine is administered intravenously, such as in co-administration with an extended release alprazolam tablet (e.g. in a unit dose strength of 0.5 mg, 1 mg, 2 mg, or 3 mg).

In certain embodiments, a GABA-agonist-like drug, such as gabapentin, may be administered intrathecally in a range of concentrations between 0.1 and 100 mg/ml. In certain embodiments, gabapentin may be delivered at a concentration between 1 and 90 mg/ml. In certain embodiments, gabapentin may be delivered at a concentration between 1 and 80 mg/ml. In addition, the daily dosage of gabapentin to be administered may depend upon the particular treatment protocol. In certain embodiments, gabapentin is administered orally, such as in the form of a tablet (available as a NEURONTIN® tablet, available from Pfizer) in the unit dose strength of 300 mg, 400 mg, 600 mg or 800 mg.

In certain embodiments, sodium valproate may be administered in the form of sodium valproate tablet (available as EPILIM® tablets from SANOFI-SYNTHELABO) having a unit dosage of 100 mg, 200 mg, or 500 mg. In certain embodiments, sodium valproate may be administered in the form of sodium valproate syrup, such as in the dose strengths of 40 mg/ml.

In certain embodiments, zolpidem may be administered in the form of Zolpidem tablets (available as STILNOX® tablets from SANOFI-SYNTHELABO) such as having a unit dosage of 5 mg or 10 mg. In certain embodiments, zaleplon may be administered in the form of Zaleplon tablets (available as SONATA® tablets from KING PHARMACEUTICALS) such as in the dose strengths of 5 mg and 10 mg.

In certain embodiments, eszopiclone may be administered in the form of Eszopiclone tablets (available as LUNESTA® tablets from SEPRACOR), e.g., having a unit dosage of 1 mg, 2 mg, or 3 mg.

In certain embodiments, sumatriptan may be administered intravenously at a dose strength of 6 mg/0.5 ml. In certain embodiments, sumatriptan may be administered in the form of sumatriptan tablets (available as IMIGRAN® tablets from GLAXO SMITH KLINE BEECHAM) such as having a unit dosage of 50 mg or 100 mg.

In certain embodiments, trazodone may be administered in the faun of Trazodone tablets (available as IMIGRAN® tablets from PLIVA), e.g., in the dose strengths of 50 mg or 100 mg

In certain embodiments, ondansetron may be administered in the form of Ondansetron tablets (available as ZOFRAN® tablets from GLAXOSMITHKLINE BEECHAM), e.g., having a unit dosage of 4 mg or 8 mg. In certain embodiments, ondansetron may be administered intravenously, such as at a dose strength of 4 mg/2 ml or 8 mg/4 ml. In certain embodiments, ondansetron may be administered in the form of ondansetron syrup, such as in the dose strengths of 4 mg/5 ml. In certain embodiments, ondansetron may be administered in the form of ondansetron wafers, such as having a unit dosage of 4 mg or 8 mg. In certain embodiments, ondansetron may be administered in the form of ondansetron suppositories, such as having a unit dosage strength of 4 mg. In certain embodiments, ondansetron may be administered in the form of ondansetron tablets (available as Zofran® tablets from Glaxosmithkline Beecham), such as in the dose strengths of 4 mg or 8 mg. In certain embodiments, ondansetron may be administered intravenously, such as at a dose strength of 4 mg/2 ml or 8 mg/4 ml. In certain embodiments, ondansetron may be administered in the form of ondansetron syrup, such as in the dose strengths of 4 mg/5 ml. In certain embodiments, ondansetron may be administered in the form of ondansetron wafers, such as in the dose strengths of 4 mg, 8 mg. In certain embodiments, ondansetron may be administered in the form of ondansetron suppositories, such as in the dose strengths of 4 mg

In certain embodiments risperidone may be administered in the form of risperidone tablets, e.g., having a unit dosage of 1 mg, 2 mg, or 4 mg.

In certain embodiments, amitrypylline may be administered in the fox in of amitrypylline tablets, such as having a unit dosage of 10 mg, 25 mg, 50 mg, or 100 mg.

In certain embodiments, chlorpromazine may be administered in the form of chlorpromazine tablets, such as having a unit dosage of 10 mg, 25 mg, or 100 mg. In certain embodiments, chlorpromazine may be administered in the form of chlorpromazine syrup, e.g., in the dose strengths of 25 mg/5 ml.

In certain embodiments, baclofen may be administered in an oral dosage form having a unit dosage of 0.25-100 mg, 0.5-80 mg, 1-60 mg, 2-50 mg, or 4-40 mg.

In certain embodiments, codeine may be administered intravenously, such as at a dose strength of 50 mg/ml. In certain embodiments, codeine may be administered in the form of codeine tablets, such as in the dose strengths of 30 mg, or 60 mg tablets.

In certain embodiments, tramadol may be administered in the form of an immediate release tramadol tablet (available as Tramal® tablets from CSL), such as in the dose strengths of 50 mg, 100 mg. In certain embodiments, tramadol may be administered in the form of a sustained release tramadol tablet, e.g., in the dose strengths of 50 mg, 100 mg, or 150 mg. In certain embodiments, tramadol may be administered intravenously, such as at a dose strength of 100 mg/2 ml.

In certain embodiments, donepezil may be administered in the form of donepezil tablets, such as in the dose strengths of 5 mg or 10 mg.

In certain embodiments, hyoscine hydrobromide may be administered in the form of hyoscine hydrobromide tablets, such as in the dose strengths of 1 mcg, 5 mcg, or 10 mcg (such as 9.9 mcg).

In certain embodiments, lamitrigene may be administered in the form of lamitrigene tablets, in the dose strengths of 2 mg, 5 mg, 25 mg, 50 mg, 100 mg, or 200 mg.

In certain embodiments, amineptine may be administered in the form of amineptine tablet (available as Ebixa® tablets from Lundback Australia Pty Ltd) such as, in the dose strengths of 10 mg. In certain embodiments, amineptine may be administered in the form of amineptine oral drops (available as Ebixa® oral drops from Lundback Australia Pty Ltd), such as in the dose strengths of 10 mg/1 ml.

In certain embodiments, clozapine may be administered in the form of clozapine tablet (available as Clopine® tablets from Myne Phauua Ltd), such as in the dose strengths of 25 mg, 50 mg, 100 mg or 200 mg.

In certain embodiments, meprobamate may be administered in the form of meprobamate Tablets (available as Meprobamate® tablets from Watson Laboratories, Inc.), such as in the dose strengths of 200 mg or 400 mg tablets.

In certain embodiments, citalopram may be administered in the form of citalopram hydrobromide (available as Citalopram® Tablets from Watson Laboratories, Inc) in the dose strengths of 10 mg, 20 mg, or 40 mg.

In certain embodiments, duloxetine may be administered in the form of duloxetine hydrochloride (available as Cymbalta® capsules from Eli Lilly and Company), such as in the dose strengths of 20 mg, 30 mg, or 60 mg.

In certain embodiments, fluoxetine may be administered in the form of fluoxetine hydrochloride (available as Prozac® capsules, delayed release capsules and oral solutions from Eli Lilly and Company) tablets such as in a dosage strength of 10 mg; capsules in a dosage strength of 10 mg, 20 mg, or 40 mg; oral solution such as in a dosage strength of 20 mg per 5 ml; and delayed-release 90 mg capsules.

In certain embodiments, fluvoxamine may be administered in the form of fluvoxamine maleate (available as Fluvoxamine Maleate® Tablets from Bay Pharma), such as in the dose strengths of 20 mg, 50 mg, or 100 mg.

In certain embodiments, Sertraline may be administered in the form of tablets (available as Zoloft® tablets, such as from Pfizer) in the dose strengths of 25 mg, 50 mg, 100 mg.

In certain embodiments, venlafaxine may be administered in the form of venlafaxine hydrochloride (available as Effexor® tablets from Wyeth Pharmaceuticals Inc.), such as in the dose strengths of 25 mg, 37.5 mg, 50 mg, 75 mg, 100 mg.

In certain embodiments, clomipramine may be administered in the form of clomipramine hydrochloride (available as Clomipramine® capsule from Mylan Pharmaceuticals Inc.), such as in the dose strengths of 25 mg, 50 mg and 75 mg.

In certain embodiments, mirtazapine may be administered in the form of mitrazapine (available as Mitrazapine® tablets from Sandoz Inc.), such as in the dose strengths of 15 mg, 30 mg and 45 mg.

In certain embodiments, bupropion may be administered in the form of bupropion (available as Bupropion SR® tablet, bupropion XL®, Wellbutrin®, Zyban® from GlaxoSmithKline Inc.), such as in the dose strengths of 75 mg, 100 mg and 150 mg.

In certain embodiments, phenelzine may be administered in the form of phenelzine (available as Nardil® tablet from Link Medical Products Pty Ltd), such as in the dose strengths of 15 mg.

In certain embodiments, moclobamide may be administered in the form of moclobamide (available as Amira® tablet from Alphapharm Pty Ltd), such as in the dose strengths of 150 mg.

In certain embodiments, doxepin may be administered in the form of tablets or capsules (available as Deptran® tablet and capsules from Alphapharm Pty Ltd), such as in the dose strengths of 10 mg, 25 mg, or 50 mg.

In certain embodiments, paraldehyde may be administered intravenously at a dose strength of 100 mcg/0.5 ml.

In certain embodiments, phenobarbital may be administered in the form of phenobarbital tablets (available as Kuniam® tablets from Pliva), such as in the dose strengths of 30 mg, 50 mg, 60 mg

In certain embodiments, phenobarbital may be administered intravenously, e.g., at a dose strength of 10 mg/kg-20 mg/kg.

In certain embodiments, clonazepam may be administered in the form of an clonazepam tablet (available as Klonopin® tablets from Roche), such as in the dose strengths of 0.25 mg, 0.5 mg, 1 mg, or 2 mg.

In certain embodiments, lorazepam may be administered in the form of an lorazepam tablet (available as Ativan® tablets from Sigma), such as in the dose strengths of 0.25 mg, 0.5 mg, 1 mg, or 2.5 mg.

In certain embodiments, carbamezapine may be administered in the form of an carbamezapine tablet (available as Tegretol® tablets from Novartis), such as in the dose strengths of 100 mg, 200 mg, 300 mg, or 400 mg.

In certain embodiments, oxcarbazepine may be administered in the form of an oxcarbazepine tablet (available as Trileptal® tablets from Novartis), such as in the dose strengths of 150 mg, 300 mg, or 600 mg.

In certain embodiments, topiramate may be administered in the form of an topiramate tablet (available as Topamax® tablets from Jannsen Cilag), such as in the dose strengths of 25 mg, 50 mg, 100 mg, or 200 mg.

In certain embodiments, pregabalin may be administered in the form of pregabalin tablet (available as Lyrica® tablets from Pfizer), such as in the dose strengths of 25 mg, 50 mg, 100 mg, 150 mg, 200 mg, 225 mg, or 300 mg

In certain embodiments, vigabatrin may be administered in the form of vigabatrin tablet (available as Sabril® tablets from Sanofi-Synthelabo), such as in the dose strength of 500 mg.

In certain embodiments, phenyloin may be administered in the form of phenyloin tablet (available as Dilantin® tablets from Pfizer), such as in the dose strengths of 30 mg or 100 mg.

In certain embodiments, ethosuximide may be administered in the form of ethosuximide tablet (available as Zarontin® tablets from Pfizer), such as in the dose strengths of 30 mg or 100 mg. In certain embodiments, lamotrigine may be administered in the form of lamotrigine tablet (available as Elmendos® tablets from GSK), such as in the dose strengths of 5 mg, 25 mg, 50 mg, 100 mg, or 200 mg.

In certain embodiments, propranolol may be administered in the form of a propranolol tablet (available as Inderal® from AstraZeneca), such as in the dose strengths of 10 mg or 40 mg.

In certain embodiments, metoprolol may be administered in the form of a metoprolol tablet (available as Betaloc® from AstraZeneca), such as in the dose strengths of 50 mg or 100 mg.

In certain embodiments, atenolol may be administered in the form of a atenolol tablet (available as Tenormin® from AstraZeneca), such as in the dose strength of 50 mg.

In certain embodiments, sotalol may be administered in the form of a sotalol tablet (available as Sotalol® from Bristol Myers Squibb), such as in the dose strengths of 80 mg or 160 mg. In certain embodiments, sotalol may be administered intravenously.

In certain embodiments, dysopyramide may be administered in the form of a dysopyramide tablet (available as Ryhtmodan® from Sanofi Aventis), such as in the dose strengths of 100 mg or 150 mg

In certain embodiments, verapamil may be administered in the form of an immediate release verapamil tablet (available as Isoptin® tablets from Abbott), such as in the dose strengths of 40 mg, 80 mg, 120 mg, or 160 mg. In certain embodiments, verapamil may be administered intravenously.

In certain embodiments, ethylenediamine may be administered in the form of a theophylline ethylenediamine tablet (available as Theo 24 and TheoCap® from Pfizer Pharmaceuticals LLC), such as in the dose strength of 300 mg.

In certain embodiments, ethanolamine may be administered in the form of carbinoxamine maleate tablets (available as Histex CT® from Wolters Kluwer Health, Inc.), such as in the dose strength of 8 mg, or 4 mg/5 ml syrup. In certain embodiments, carbinoxamine maleate may be administered in the form of capsules (available as Histex I/E from Wolters Kluwer Health, Inc.), such as in the dose strengths of 2 mg or 8 mg. In certain embodiments, carbinoxamine maleate may be administered in the form of a syrup, such as at a dose strength of 4 mg/5 ml.

In certain embodiments, acrivastine may be administered in the form of capsules (available as Semprex-D Capsules® from DSM Pharmaceuticals, Inc.), such as in the dose strength of 8 mg.

In certain embodiments, antazolin may be administered in the form of an antazolin sulfate eye drop (available as Antistine Privine Eye Drops® from Novartis Pharmaceuticals Australia Pty Ltd), such as in the dose strength of 5 mg/mL.

In certain embodiments, diphenhydramine may be administered in the form of diphenhydramine hydrochloride capsules (available as Diphenhydramine Hydrochloride Capsules® from Barr Laboratories, Inc.), such as in the dose strength of 50 mg.

In certain embodiments, doxylamine may be administered in the form of doxylamine tablets (available as Doxylamine Tablets® from Cerner Multum, Inc), such as in the dose strengths of 25 mg or 50 mg.

In certain embodiments, clemastine may be administered in the form of clemastine fumarate tablets or capsules (available as Clemastine Fumarate® from Sandoz Inc.), such as in the dose strengths of 1 mg, 5, mg, or 10 mg (such as 2.68 mg).

In certain embodiments, dimenhydrinate may be administered in the form of dimenhydrinate tablets (available as Dimenhydrinate® from Johnson & Johnson Pacific Pty Limited), such as in the dose strength of 50 mg.

In certain embodiments, chlorpheniramine may be administered in the form of chlorpheniramine maleate tablets (available as Aller-Chlor tablet from Wolters Kluwer Health, Inc), such as in the dose strength of 4 mg.

In certain embodiments, dexchlorpheniramine may be administered in the form of dexchlorpheniramine maleate tablets (available as Dexchlorpheniramine Maleate tablet from Morton Grove Pharmaceuticals, Inc.), such as in the dose strengths 4 mg or 6 mg.

In certain embodiments, brompheniramine may be administered in the form of brompheniramine tablets (available as Bidhist®), such as in the dose strength of 6 mg.

In certain embodiments, promethazine may be administered in the form of promethazine hydrochloride tablets (available as Phenergon® from ANI Pharmaceuticals, Inc.), such as in the dose strength of 25 mg.

In certain embodiments, piperazine may be administered in the form of piperazine estrone sulfate tablets (available as Ogen® tablets from Abbott Laboratories), such as in the dose strengths of 0.625 mg, 1.25 mg, or 2.5 mg.

In certain embodiments, mexiletine may be administered in the form of a tablet (available as Mexitil® from Boelhringer Ingelheim) such as in the dose strengths of 50 mg or 200 mg.

In certain embodiments, tocamide may be administered in the form of a tablet (available as Tonocard® from Eli Lilly) such as in the dose strengths of 400 mg or 800 mg.

In certain embodiments, memantine may be administered in the fowl of a tablet (available as Namenda® from Forest Laboratories) such as in the dose strengths of 5 mg or 10 mg.

In certain embodiments, dexamethasone may be administered in the form of a tablet (available as Dexmethasone® from Aspen) such as in the dose strengths of 0.5 mg, 0.75 mg, 1.0 mg, 1.5 mg, 2 mg, 4 mg, or 6 mg.

In certain embodiments, dexamethasone may be administered in the form of a ear drops (available as Otodex® from Sanofi-Aventis) such as in the dose strength of 0.5 mg/ml.

In certain embodiments, prednisone may be administered in the form of a tablet (available as Predsone® from Aspen) such as in the dose strengths of 1 mg, 2.5 mg, 20 mg, or 50 mg.

In certain embodiments, furosemide may be administered in the form of a tablet (available as Lasix® from Sanofi-Aventis) such as in the dose strengths of 20 mg, 40 mg, or 80 mg.

In certain embodiments, misoprostol may be administered in the form of a tablet (available as Cytotec® from Pfizer) such as in the dose strengths of 100 mcg or 200 mcg.

In certain embodiments, rivastigmine may be administered in the form of a tablet (available as Exelon® from Novartis) such as in the dose strengths of 1.5 mg, 3.0 mg, or 6.0 mg.

In certain embodiments, galantamine may be administered in the form of a tablet (available as Reminyl® from Janssen Cilag) such as in the dose strengths of 8 mg, 16 mg, or 24 mg.

In certain embodiments, dextroamphetamine may be administered in the faun of a tablet (available as Dexedrine® from GlaxosmithKline Beecham) such as in the dose strengths of 8 mg, 16 mg, or 24 mg.

In certain embodiments, methylphenidate may be administered in the form of a tablet (available as Ritalin® from Novartis) such as in the dose strengths of 5 mg, or 10 mg.

In certain embodiments, acamprosate may be administered in the form of a tablet (available as Campral® from Forest) such as in the dose strengths of 5 mg, or 10 mg.

In certain embodiments, Midazolam may be administered in the form of a tablet (available as Midazolam® from Sandoz) such as in the dose strengths of 1 mg/1 ml, or 5 mg/1 ml.

Where two or more pharmaceutical agents are administered, they may be administered in either a single formulation or in separate pharmaceutical compositions or formulations (such as in two or more, three or more, or even four or more separate pharmaceutical compositions or formulations). Moreover, the two or more pharmaceutical agents can be administered to the subject at essentially the same time, i.e., concurrently, or at separately staggered times, i.e., sequentially. In certain embodiments, one or more pharmaceutical agents are administered sequentially (e.g., prior to or after) to two or more other pharmaceutical agents that are administered simultaneously.

Administration of the one or more pharmaceutical agents by the routes mentioned herein using the regimens disclosed may be suitable as long as the beneficial pharmaceutical effect of the pharmaceutical agent(s) is realized by the subject and/or an appropriate number of subjects.

The appropriate dosing regimen utilizing the pharmaceutical agent(s), the amount of each dose administered, and the intervals between doses of the agents will depend on various factors such as the particular pharmaceutical agent(s) being used, the type of pharmaceutical formulation(s) being used, the type of auditory system disorder (such as the type of tinnitus) being treated, the characteristics of the subject being treated (e.g., species, age, weight, sex, medical condition, fed/fasted), the route of administration, and the severity of the disorder being treated. A physician of ordinary skill can readily determine and prescribed the effective amount of the pharmaceutical composition to prevent or to treat the specific physiological condition.

Such compositions may be administered in a single daily dose, or the total daily dosage may be administered in divided doses several times daily. Furthermore, the pharmaceutical compositions may be administered as a single dose or over a period of time. Additionally, the pharmaceutical composition can be administered continuously or intermittently. The daily dosage may be varied over wide range and can be such that the amount of the pharmaceutical agent is sufficient to cause its desired effects.

The composition or formulation to be administered will contain a quantity of the compounds or pharmaceutically acceptable salts thereof in an amount effective to treat the auditory system disorder of the subject being treated. Because two or more pharmaceutical agents are being used together in some embodiments, the potency of each of the agents and the interactive effects achieved by combining them together must also be taken into account. Moreover, because the pharmaceutical agent(s) are being used together with one or more auditory system disorder devices, a subject's physiological response to the device must be taken into account with the potency of each of the pharmaceutical agent(s). A consideration of these factors is within the purview of the ordinarily skilled clinician for the purpose of determining the therapeutically effective or prophylactically effective dosage amounts needed to improve one or more symptoms of an auditory system disorder.

The one or more pharmaceutical agents are administered concurrently with, or in combination with (such as co-administered with), or in conjunction with at least one auditory system disorder device, in various suitable manners (such as separately, sequentially, and/or simultaneously). In certain embodiments, an effective amount of one or more pharmaceutical agents may be administered prior to the start of administering of at least one auditory system disorder device, such as at least about 24 hours (such as at least about 18 hours, at least about 12 hours, at least about 8 hours, at least about 6 hours, at least about 5 hours, at least about 4 hours, at least about 3 hours, at least about 2 hours, at least about 1 hour, at least about 45 minutes, at least about 30 minutes, at least about 20 minutes, at least about 10 minutes, at least about 5 minutes, at least about 1 minutes, or even at least about 30 seconds) prior to the start of administering of at least one auditory system disorder device. In certain embodiments, an effective amount of one or more pharmaceutical agents may be administered simultaneously with the start of administering of one or more auditory devices to a subject, such as within about 20 minutes, about 10 minutes, about 5 minutes, about 1 minutes, about 30 seconds, or within even about 5 seconds of the start of administering of at least one auditory system disorder device. In certain embodiments, an effective amount of one or more pharmaceutical agents may be administered after the start of administering of at least one auditory system disorder device, such as less than about 10 hours (such as less than about 8 hours, about 6 hours, about 4 hours, about 2 hours, about 1 hour, about 45 minutes, about 30 minutes, about 20 minutes, about 10 minutes, about 5 minutes, about 1 minute, or even less than about 30 seconds) after the start of administering of at least one auditory system disorder device to a subject. In certain embodiments involving repeated (e.g., daily, or several times per day) use of an acoustic device, an effective amount of one or more pharmaceutical agents may be administered over a period that precedes, overlaps or follows a period of use of the device. For example, the pharmaceutical agent may be used for a day or several days prior to commencement of regular device use, and/or its use maintained for several days, weeks or months while an acoustic device is being used.

Patient compliance may be a factor in receiving a good result in medical treatment. Causes for poor compliance may include, but are not limited to, complicated regimen, unattractive and/or painful treatments, memory challenges in administering two or more distinct components of non-combinative therapy regimen, and/or physical difficulty in complying. Therefore, separate administration of the pharmaceutical agent(s) and auditory system disorder device(s) may not be convenient or satisfactory to achieve the most optimal results. The present embodiments may provide improved patient compliance in patients having at least one auditory system disorder, such as an increase of at least about 5% (such as at least about 10%, at least about 15%, at least about 20%, at least about 30%, at least about 40%, at least about 50%, at least about 60%, at least about 70%, at least about 80%, at least about 85%, at least about 90%, or even at least about 95%) in patient compliance (such as with proper administering procedures), relative to patient compliance in patients having the auditory system disorder and receiving separate administrations of a pharmaceutical agent and auditory system device in a non-combinative therapy regimen, such as determined via survey results.

Certain embodiments may provide improved relief of one or more symptoms of an auditory system disorder in patients having the auditory system disorder, such as an improvement of at least about 5% (such as at least about 10%, at least about 15%, at least about 20%, at least about 30%, at least about 40%, at least about 50%, at least about 60%, at least about 70%, at least about 80%, at least about 85%, at least about 90%, or even at least about 95%) in relief of one or more symptoms of the auditory system disorder, relative to symptom relief in patients the same auditory system disorder and receiving separate administrations of a pharmaceutical agent and auditory system device in a non-combinative therapy regimen.

Some embodiments may allow for a lessening, shortening, and/or decreasing of the total daily administering time (or treatment duration or time) of an auditory system disorder device, needed to achieve a desired level of relief of one or more symptoms of an auditory system disorder (such as perceived loudness of tinnitus) within a subject (having an auditory system disorder), relative to the daily administering time required to achieve comparable (or the same) symptom relief for the same subject (or a subject having the same auditory system disorder) through administering of the auditory system disorder device alone (with no pharmaceutical agent). Certain embodiments may allow for the achievement of the same or better relief (e.g., at least the same relief) of one or more symptoms of an auditory system disorder within a subject (having an auditory system disorder) (or a statistically significant population of subjects) than the symptom relief that is achievable through administering of an auditory system disorder device alone (with no pharmaceutical agent), certain embodiments may require at least 5% less (such as at least 10%, at least 15%, at least 20%, at least 30%, at least 40%, at least 50%, at least 60%, at least 70%, at least 80%, at least 85%, at least 90%, or even at least 95% less) daily administering time (or treatment time) with the auditory system disorder device, relative to the daily administering time (or treatment time) required to attain or achieve comparable (or the same) symptom relief for the same subject (or a subject having the same auditory system disorder) (or population of subjects) when using the auditory system disorder device alone (with no pharmaceutical agent), such as determined via survey results.

In exemplary embodiments, the administering time per day may be about 2-4 hours, about 3-5 hours, about 4-6 hours, about 5-7 hours, about 1 hour, about 2 hours, about 3 hours, about 4 hours, about 5 hours, etc. In exemplary embodiments, the administering time may be reduced by about 1 hour about 2 hours, about 3 hours, about 4 hours, about 5 hours, etc.

Certain embodiments may allow for the achievement of the same or better (e.g., at least the same) relief of one or more symptoms of an auditory system disorder than that which is achievable through administering of a pharmaceutical agent alone (with no auditory system disorder device). Certain embodiments may require at least a 5% lower (such as at least 10%, at least 15%, at least 20%, at least 30%, at least 40%, at least 50%, at least 60%, at least 70%, at least 80%, at least 85%, at least 90%, or even at least 95% lower) daily dosage of the pharmaceutical agent, relative to the daily dosage of the pharmaceutical agent required to attain or achieve comparable (or the same) symptom relief for the same subject (or a subject having the same auditory system disorder) when using the pharmaceutical agent alone (with no auditory system disorder device).

Certain embodiments may allow for the achievement of desired symptom relief for a subject while reducing, lessening, minimizing, preventing, and/or eliminating side effects associated with normal prescribed use of the pharmaceutical agent alone. Certain embodiments achieve at least a 5% lower (such as at least 10%, at least 15%, at least 20%, at least 30%, at least 40%, at least 50%, at least 60%, at least 70%, at least 80%, at least 85%, at least 90%, or even at least 95% lower) incidence of side effects associated with the pharmaceutical agent, relative to the normal incidence of side effects within the same subject (or population of subjects) being administered an effective amount of the pharmaceutical agent alone, such as determined by survey results.

Suitable treatment periods may be utilized in treating a subject. In certain embodiments, the pharmaceutical agent(s) and auditory system disorder device(s) are administered to the subject on an as-needed basis for a period of at least about 2 days, such as at least about 1 week, at least about 2 weeks, at least about 1 month, at least about 2 months, at least about 6 months, at least about 1 year, at least about 2 years, or even at least about 5 years.

Administration of an effective amount of the pharmaceutical agent(s) and auditory system disorder device(s) to the subject includes both self-administration and administration to the subject by another person (e.g., physician, health care provider, or pharmaceutical or device supplier).

Certain disclosed embodiments include methods for improving one or more symptoms associated with an auditory system disorder in a subject diagnosed with the auditory system disorder, the method comprising administering to the subject (a) an effective amount of at least one pharmaceutical agent and (b) at least one auditory system disorder device.

The present disclosure is also directed to articles of manufacture such as kits that include the pharmaceutical agent(s) as well as the auditory system disorder device(s) packaged for distribution. Kits can additionally include instructions for using the kit components with disclosed methods. Thus, for example, a kit can include a pharmaceutical agent (such as an anxiolytic agent or an antihistamine agent) and an auditory system disorder device (such as an Oasis™ Device, available from Neuromonics, Inc. (Bethlehem, Pa.)) in a container or pack, together with instructions for administration to a human subject. Instructions can include instructions for practicing the methods described herein. Instructions may additionally include indications of a satisfactory clinical endpoint or any adverse symptoms that may occur, or any additional information required by, for example, the Food and Drug Administration for use in humans. Instructions may additionally include written materials that are functionally related to the kit and necessary for proper usage of the kit, such as, for example, contraindications, potential side effects, and/or adverse events information relating to the pharmaceutical agent(s).

Kits can additionally include a buffering agent, a preservative, or a stabilizing agent for the pharmaceutical agent. Each component of the kit can be enclosed within an individual container and all of the various containers can be within a single package.

Packaging can be accomplished by a number of means utilized in the pharmaceutical industry. Examples of such packaging are: unit dose containers for dispensing liquid compositions enclosed in a box or container along with one or more auditory system disorder devices and with package inserts; plastic and/or foil wrappers holding solid ocular inserts which contain the pharmaceutical agent(s) and which are enclosed in a box or container along with one or more auditory system disorder devices and package inserts. Other modes of packaging would be readily apparent to one skilled in the pharmaceutical packaging arts.

In some embodiments, methods for using the pharmaceutical agent(s) and the auditory system disorder device(s) in the manufacture of a system or kit for use in treating an auditory system disorder are provided.

Administration of at least one pharmaceutical agent simultaneously in combination with at least one auditory system disorder device, prior to or following the administration of an auditory system disorder device serves to improve one or more symptoms associated with an auditory system disorder, such as primary and/or secondary sequelae or symptoms associated with the auditory system disorder, such as psychological, perceptual, attentional, and/or reactionary sequelae associated with the auditory system disorder, and/or symptoms associated with any neuroplastic changes associated with, causing, and/or related to auditory system disorder, such as neuroplastic changes in the auditory system that leads to an initial perception of an auditory system disorder (such as tinnitus) sound by a subject, in attentional filters in the brain that cause the subject to pay attention to the auditory system disorder (such as tinnitus) perception, and/or in regions of the limbic, emotional, and/or autonomic nervous system that cause an aversive reaction to auditory system disorder symptoms. Such symptoms of an auditory system disorder include, for example, reduced sound tolerance, including hyperacusis, and/or perception of sounds that are not present in the external environment and/or that other people cannot generally hear, such as, for example, ringing in the ears, clicking, beating, pounding, buzzing and humming background sounds, roaring, or whistling noises in the ears.

The presence, nature, severity, or degree of one or more symptom(s) of an auditory system disorder of a subject can be assessed, quantified, and/or determined through methods available to those of skill in the art, such as through any objective and/or subjective assessment test or device. In this manner, improvements in one or more symptoms associated with an auditory system disorder can be assessed and/or quantified by comparing the existence, level, and/or degree of auditory system disorder symptom(s) quantified for a subject (or group of subjects) during and/or after treatment, with the existence, level, and/or degree of auditory system disorder symptom(s) quantified for the same subject (or group of subjects) prior to such treatment, or during and/or after treatment with one or more auditory system disorder devices (and no pharmaceutical agent(s)), or during and/or after treatment with one or more pharmaceutical agents (and no auditory system disorder device(s)), and/or prior to treatment.

In certain embodiments, the nature of the auditory system disorder may be assessed via audiogram (such as pure tone audiogram) or other hearing acuity test.

In certain embodiments, reduction in perceived loudness of tinnitus may be quantified, determined, and/or assessed. In particular, for example, treatments can reduce perceived tinnitus loudness by at least about 5% (such as at least about 10%, about 15%, about 20%, about 40%, about 60%, about 80%, about 100%, about 120%, about 140%, about 180%, or even at least about 200%), as compared to the level of loudness detected (i) prior to treatment (or with no treatment), (ii) during treatment of the subject with an auditory system disorder device only, and/or (iii) during treatment of the subject with a pharmaceutical agent only. Perceived loudness can be assessed and quantified by methods available to those of skill, such as through use of a Loudness Match test. Such a test can be performed, for example, by adjusting the loudness of an external tone that most closely matches the tone of the subject's tinnitus. The test proceeds until the external tones are as loud as the tone associated with the subject's tinnitus.

In certain embodiments, improvement in awareness of a subject's auditory system disorder (such as tinnitus) may be quantified, determined, and/or assessed by determination of a change in the percent of time that the patient is generally aware of their auditory system disorder. In particular, for example, treatment with certain embodiments can reduce awareness such as at least about 5%, 10%, 15%, 20%, 40%, 60%, 80%, 100%, 120%, 140%, 180%, or even at least 200% as compared to the level of maskability achieved during treatment of the subject with only an auditory system disorder device.

In certain embodiments, improvement in maskability of a subject's auditory system disorder (such as tinnitus) may be quantified, determined, and/or assessed by determination of a change in maskability of the auditory system disorder. In particular, for example, treatment with certain embodiments can increase maskability by at least about 5% (such as at least about 10%, about 15%, about 20%, about 40%, about 60%, about 80%, about 100%, about 120%, about 140%, about 180%, or even at least about 200%), as compared to the level of maskability achieved during treatment of the subject with only an auditory system disorder device. Maskability of the auditory system disorder of a subject can be assessed and quantified by methods available to those skill in the art, such as through use of a Maskability test. Such a test can be performed, for example, by delivering external sounds (such as a band of noise extending from 2,000 to 12,000 Hz) through headphones to the affected ear of a subject. During this test, the masking sound is increased gradually until its presence is detected. Then the intensity of the sound is further increased until the person can no longer hear a tone associated with auditory system disorder in that ear. The minimum masking level (MML) may be expressed in dB sensation level (SL).

In certain embodiments, improvements in symptom(s) associated with a subject's auditory system disorder may be quantified, determined, and/or assessed by determination of a change in the internal auditory system disorder spectrum for the subject, as compared with the internal auditory system disorder spectrum measured for the subject (i) prior to treatment (or with no treatment), (ii) during treatment with an auditory system disorder device only, and/or (iii) during treatment of the subject with a pharmaceutical agent only. An internal spectrum of a subject's auditory system disorder (such as tinnitus) can be assessed and quantified by methods known to those skill in the art, such as by asking subjects diagnosed with an auditory system disorder to rate on a numeric scale the contribution of elementary pitch sensations evoked by isolated frequency components to their overall auditory system disorder sensation. The resulting ‘internal auditory system disorder spectra’ may be viewed as representing the estimated perceptual contribution to an overall auditory system disorder (such as tinnitus) sensation for a subject, as a function of frequency over a large range of frequencies. In a large number of cases, the measured ‘internal auditory system disorder spectra’ may be found to occupy a wide frequency range corresponding largely to those frequencies at which hearing thresholds are abnormally elevated. In most cases, they exhibit a broad peak falling within the hearing loss range.

There are several quality of life (QOL) instruments or assessments that can be used to assess the impact that the disclosed treatments have on different aspects of a subject's functioning and well being. Other ways of measuring or assessing the lessening of symptoms of an auditory system disorder are available and will be known to those of skill in the art and, if appropriate, may be used. For example, the impact of tinnitus on the patient's quality of life may be assessed using such tools as the Tinnitus Reaction Questionnaire, Tinnitus Handicap Questionnaire, Tinnitus Handicap Inventory, or Tinnitus Questionnaire. The impact of hyperacusis on quality of life may be assessed using such tools as the Hyperacusis Questionnaire.

### EXAMPLES

The following examples demonstrate the advantages of administering to a subject diagnosed with an auditory system disorder at least one pharmaceutical agent and at least one auditory system disorder device, in combination or sequentially.

### Example 1

### Prophetic Comparative

A subject having been diagnosed with the auditory system disorder tinnitus is chosen. The subject reports difficulty relaxing as a result of their tinnitus. The nature and severity of the subject's tinnitus are assessed via Loudness Match Test, Awareness Test, Maskability Test, Residual Inhibition Test, Pitch Match Test, and Tinnitus Reaction Questionnaire.

The subject is then orally administered one tablet per day of immediate release 0.5 mg alprazolam. The subject is instructed to repeat administration of this pharmaceutical agent daily and to return for clinical re-evaluation after two months.

The nature and severity of the subject's tinnitus are re-evaluated after two months using the same test(s) used prior to treatment.

### Example 2

### Prophetic Comparative

The nature and severity of the tinnitus of the subject of Example 1 are assessed via Loudness Match Test, Awareness Test, Maskability Test, Residual Inhibition Test, Pitch Match Test, and Tinnitus Reaction Questionnaire.

The subject is administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

### Example 3

### Prophetic

The nature and severity of the tinnitus of the subject of Example 1 are assessed via Loudness Match Test, Awareness Test, Maskability Test, Residual Inhibition Test, Pitch Match Test, and Tinnitus Reaction Questionnaire.

The subject is then orally administered one tablet per day of immediate release 0.5 mg alprazolam, and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with alprazolam only in Example 1.

Data are also expected to show improvement in the severity and degree of at least one symptom of the subject's tinnitus as compared to the assessed severity and degree of the subject's symptoms after treatment with the Oasis™ Device only in Example 2.

### Example 4

### Prophetic

A subject having been diagnosed with the auditory system disorder tinnitus is chosen. The subject reports depression in association with their tinnitus.

The nature and severity of the tinnitus of the subject are assessed via Loudness Match Test, Awareness Test, Maskability Test, Residual Inhibition Test, Pitch Match Test, and Tinnitus Reaction Questionnaire.

The subject is then orally administered one tablet per day of 50 mg of sertraline and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with sertraline only.

Data are also expected to show improvement in the severity and degree of at least one symptom of the subject's tinnitus as compared to the assessed severity and degree of the subject's symptoms after treatment with the Oasis™ Device only.

### Example 5

### Prophetic

A subject having been diagnosed with the auditory system disorder tinnitus is chosen. The subject reports anxiety associated with their tinnitus.

The nature and severity of the tinnitus of the subject are assessed via Loudness Match Test, Awareness Test, Maskability Test, Residual Inhibition Test, Pitch Match Test, and Tinnitus Reaction Questionnaire.

The subject is then orally administered one tablet per day of 5 mg of diazepam and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with diazepam only.

Data are also expected to show improvement in the severity and degree of at least one symptom of the subject's tinnitus as compared to the assessed severity and degree of the subject's symptoms after treatment with the Oasis™ Device only.

### Example 6

### Prophetic

A subject having been diagnosed with the auditory system disorder tinnitus is chosen. The subject is found to exhibit a significant degree of hearing loss and reports prominent and disturbing tinnitus. The subject does not appear to exhibit significant emotional distress, nor does the subject report significant stress in their life.

The nature and severity of the subject's tinnitus are assessed via Loudness Match Test, Awareness Test, Maskability Test, Residual Inhibition Test, Pitch Match Test, and Tinnitus Reaction Questionnaire.

The subject is then orally administered one tablet per day of immediate release 1000 mg vigabatrin, and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with vigabatrin only.

Data are also expected to show improvement in the severity and degree of at least one symptom of the subject's tinnitus as compared to the assessed severity and degree of the subject's symptoms after treatment with the Oasis™ Device only.

### Example 7

### Prophetic

A subject having been diagnosed with the auditory system disorder tinnitus is chosen. The subject is found to experience difficulty diverting their conscious attention away from their tinnitus.

The nature and severity of the subject's tinnitus are assessed via Loudness Match Test, Awareness Test, Maskability Test, Residual Inhibition Test, Pitch Match Test, and Tinnitus Reaction Questionnaire.

The subject is then orally administered one tablet per day of immediate release 2.5 mg dextroamphetamine, and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with dextroamphetamine only.

Data are also expected to show improvement in the severity and degree of at least one symptom of the subject's tinnitus as compared to the assessed severity and degree of the subject's symptoms after treatment with the Oasis™ Device only.

### Example 8

### Prophetic

A subject having been diagnosed with the auditory system disorder tinnitus is chosen.

The nature and severity of the tinnitus of the subject are assessed via Loudness Match Test, Awareness Test, Maskability Test, Residual Inhibition Test, Pitch Match Test, and Tinnitus Reaction Questionnaire.

The subject is then orally administered one tablet per day of 400 mg of carbamezapine and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with carbamezapine only.

Data are also expected to show improvement in the severity and degree of at least one symptom of the subject's tinnitus as compared to the assessed severity and degree of the subject's symptoms after treatment with the Oasis™ Device only.

### Example 9

### Prophetic

A subject having been diagnosed with the auditory system disorder tinnitus is chosen. The subject is found to experience vertigo associated with tinnitus.

The nature and severity of the tinnitus of the subject are assessed via Loudness Match Test, Awareness Test, Maskability Test, Residual Inhibition Test, Pitch Match Test, and Tinnitus Reaction Questionnaire.

The subject is then orally administered one tablet per day of 4 mg of carbinoxamine maleate and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with carbinoxamine only.

Data are also expected to show improvement in the severity and degree of at least one symptom of the subject's tinnitus as compared to the assessed severity and degree of the subject's symptoms after treatment with the Oasis™ Device only.

### Example 10

### Prophetic

A subject having been diagnosed with the auditory system disorder tinnitus is chosen. The subject is found to suffer from drug-induced tinnitus.

The nature and severity of the tinnitus of the subject are assessed via Loudness Match Test, Awareness Test, Maskability Test, Residual Inhibition Test, Pitch Match Test, and Tinnitus Reaction Questionnaire.

The subject is then orally administered one tablet per day of 80 mg of verapamil and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with verapamil only.

Data are also expected to show improvement in the severity and degree of at least one symptom of the subject's tinnitus as compared to the assessed severity and degree of the subject's symptoms after treatment with the Oasis™ Device only.

### Example 11

### Prophetic

A subject having been diagnosed with the auditory system disorder of hyperacusis is chosen. The subject reports anxiety associated with their hyperacusis.

The nature and severity of the tinnitus of the subject are assessed via Loudness Match Test, Awareness Test, Maskability Test, Residual Inhibition Test, Pitch Match Test, and Tinnitus Reaction Questionnaire.

The subject is then orally administered one tablet per day of 100 mg of lamotrigene and instructed to repeat administration of this pharmaceutical agent daily for a period of two months. The subject is also administered an Oasis™ Device (available from Neuromonics, Inc. (Bethlehem, Pa.)) and instructed in its appropriate use for at least two hours per day. The subject is instructed to use the device daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with lamotrigene only.

Data are also expected to show improvement in the severity and degree of at least one symptom of the subject's tinnitus as compared to the assessed severity and degree of the subject's symptoms after treatment with the Oasis™ Device only.

### Example 12

### Prophetic

A subject having been diagnosed with the auditory system disorder tinnitus is chosen. The subject is found to exhibit a significant degree of hearing loss and reports prominent and disturbing tinnitus. The subject does not appear to exhibit significant emotional distress, nor does the subject report significant stress in their life.

The nature and severity of the subject's tinnitus are assessed via Loudness Match Test, Awareness Test, Maskability Test, Residual Inhibition Test, Pitch Match Test, and Tinnitus Reaction Questionnaire.

The subject is then orally administered one tablet per day of immediate release 1000 mg vigabatrin, and instructed to repeat administration of this pharmaceutical agent daily for a period of two months. The subject is also administered a hearing aid and instructed in its appropriate use. The subject is instructed to use the hearing aid daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with vigabatrin only.

Data are also expected to show improvement in the severity and degree of at least one symptom of the subject's tinnitus as compared to the assessed severity and degree of the subject's symptoms after treatment with the hearing aid only.

### Example 13

### Prophetic

A subject having been diagnosed with the auditory system disorder tinnitus is chosen. The subject is found to exhibit a significant degree of hearing loss and reports prominent and disturbing tinnitus. The subject does not appear to exhibit significant emotional distress, nor does the subject report significant stress in their life.

The nature and severity of the subject's tinnitus are assessed via Loudness Match Test, Awareness Test, Maskability Test, Residual Inhibition Test, Pitch Match Test, and Tinnitus Reaction Questionnaire.

The subject is then orally administered one tablet per day of immediate release 1000 mg vigabatrin, and instructed to repeat administration of this pharmaceutical agent daily for a period of two months. The subject is also administered a tinnitus masker and instructed in its appropriate use. The subject is instructed to use the hearing aid daily and return for clinical re-evaluation after two months.

The nature and severity of the subject's tinnitus are re-evaluated after two months using the same test(s) used prior to treatment.

Data are expected to show improvement in the severity and degree of at least one symptom of the subject's tinnitus after two months of treatment, as compared to the assessed severity and degree of the subject's symptoms prior to the treatment period.

Data are also expected to show improvement in the severity and degree of at least one symptom of the subject's tinnitus as compared to the assessed severity and degree of the subject's symptoms after treatment with vigabatrin only.

Data are also expected to show improvement in the severity and degree of at least one symptom of the subject's tinnitus as compared to the assessed severity and degree of the subject's symptoms after treatment with the tinnitus masker only.

1. A kit for improving one or more symptoms associated with an auditory system disorder in a subject diagnosed with the auditory system disorder, comprising:
(a) an effective amount of at least one pharmaceutical agent; and
(b) at least one auditory system disorder device.

2. The kit of claim 1, wherein the pharmaceutical agent comprises one or more anxiolytic agents, antidepressant agents, anticonvulsants, antiarrthymic agents, antihistamines, muscarinic agents, Hi antagonists, opioid agents, osmotic regulators, vasodilators, anesthetic agents, N-methyl-D-aspartic acid (NMDA) receptor antagonists, diuretics, or combinations or mixtures thereof.

3. The kit of any of claim 1, wherein at least one pharmaceutical agent comprises alprazolam.

4. The kit of claim 3, wherein the effective amount comprises about 0.25-4 mg of alprazolam.

5. The kit of claim 1, wherein at least one auditory system disorder device is a masking device, a hearing aid, a cochlear implant, sound generator, music player and/or a tinnitus retraining device.

6. The kit of claim 1, wherein at least one auditory system disorder device is an audio playback device that comprises a receiver configured to receive an audio signal, a processor for spectrally modifying the audio signal in a substantially real time manner to compensate for an auditory system disorder, and an output for outputting the spectrally modified audio signal to the subject.

7. The kit of claim 1, wherein at least one auditory system disorder device is configured to provide a treatment signal and to modify a treatment signal to include troughs and peaks wherein the modified treatment signal intermittently masks the auditory system disorder of the subject during at least one of the peaks and does not fully mask the auditory system disorder during at least some of the troughs.

8. The kit of claim 1, wherein at least one auditory system disorder device is configured to provide a relaxing stimulus on at least one of a psychological, acoustic, and neural level.

9. The kit claim 1, wherein the kit provides at least about 5% greater relief of one or more symptoms of the auditory system disorder, relative to symptom relief in patients having the same auditory system disorder and receiving separate administrations of a pharmaceutical agent and auditory system device in a non-combinative therapy regimen.

10. The kit of claim 1, wherein the effective amount of the at least one pharmaceutical agent in the kit comprises at least a 5% lower daily dosage of the pharmaceutical agent, as compared to the daily dosage of the pharmaceutical agent required to achieve comparable symptom relief in a subject having the same auditory system disorder and being administered the at least one pharmaceutical agent alone.

11. The kit of claim 1, wherein the kit allows for the achievement of at least the same relief of one or more symptoms of the auditory system disorder within the subject with at least 5% less daily administering time of the auditory system disorder device, as compared to the daily administering time required to achieve said symptom relief for a subject having the same auditory system disorder and using the auditory system disorder device alone.

12. The kit claim 1, wherein use of the kit by the subject achieves said improvements in one or more symptoms with at least 5% less incidence of side effects associated with the at least one pharmaceutical agent, as compared to the normal incidence of side effects within subjects being administered an effective amount of the pharmaceutical agent alone.

13. The kit of any of claim 1, wherein the kit achieves at least about 5% greater compliance with proper administering procedures in subjects having at least one auditory system disorder, relative to compliance in subjects having the auditory system disorder and receiving separate administrations of a pharmaceutical agent and auditory system device in a non-combinative therapy regimen.

14. A combination therapy for improving one or more symptoms associated with an auditory system disorder in a subject diagnosed with the auditory system disorder, comprising:
(a) an effective amount of at least one pharmaceutical agent and (b) at least one auditory system disorder device.

15. The combination therapy of claim 1, wherein the pharmaceutical agent comprises one or more anxiolytic agents, antidepressant agents, anticonvulsants, antiarrthymic agents, antihistamines, muscarinic agents. Hi antagonists, opioid agents, osmotic regulators, vasodilators, anesthetic agents. ISIMDA receptor antagonists, diuretics, or combinations or mixtures thereof.

16. A method for improving one or more symptoms associated with an auditory system disorder in a subject diagnosed with the auditory system disorder, said method comprising administering to said subject:
(a) an effective amount of at least one pharmaceutical agent; and
(b) at least one auditory system disorder device.

17. The method of claim 16, wherein said at least one auditory system disorder device is administered for an effective duration.

18. The method of claim 16, wherein at least one pharmaceutical agent and at least one auditory system disorder device are administered simultaneously.

19. The method of claim 6, wherein at least one pharmaceutical agent and at least one auditory system disorder device are administered sequentially.

20. The method of claim 16, wherein the pharmaceutical agent comprises one or more anxiolytic agents, antidepressant agents, anticonvulsants, antiarrthymic agents, antihistamines, muscarinic agents. Hi antagonists, opioid agents, osmotic regulators, vasodilators, anesthetic agents. NMDA receptor antagonists, diuretics, or combinations or mixtures thereof.

21. The method of claim 16, wherein at least one auditory system disorder device is a masking device, a hearing aid, a cochlear implant, sound generator, music player and/or a tinnitus retraining therapy device.

22. The method of claim 16, wherein at least about 5% greater relief of the one or more symptoms of the auditory system disorder is achieved in the subject, as compared to the symptom relief achieved in subjects having the same auditory system disorder and receiving separate administrations of a pharmaceutical agent and auditory system device in anon-combinative therapy regimen.

23. The method of claim 16, wherein the effective amount of the at least one pharmaceutical agent comprises at least a 5% lower daily dosage of the pharmaceutical agent, as compared to the daily dosage of the pharmaceutical agent required to achieve comparable symptom relief in a subject having the same auditory system disorder and being administered the at least one pharmaceutical agent alone.

24. The method of claim 16, wherein the method achieves at least the same relief of one or more symptoms of the auditory system disorder within the subject with at least 5% less daily administering time of the auditory system disorder device, as compared to the daily administering time required to achieve said symptom relief for a subject having the same auditory system disorder and using the auditory system disorder device alone.

25. The method of claim 16, wherein said improvements in one or more symptoms of the subject are achieved with at least 5% less incidence of side effects associated with the at least one pharmaceutical agent, as compared to the normal incidence of side effects within subjects being administered an effective amount of the pharmaceutical agent alone.

26. The method of claim 16, wherein at least about 5% greater compliance with proper administering procedures is achieved in the subject, relative to compliance achieved in subjects having the auditory system disorder and receiving separate administrations of a pharmaceutical agent and auditory system device in a non-combinative therapy regimen.

27. A method for improving one or more symptoms associated with an auditory system disorder in a subject diagnosed with the auditory system disorder, said method comprising administering to said subject (a) an effective amount of at least one pharmaceutical agent selected from anxiolytic agents, antidepressant agents, and mixtures thereof, and (b) an audio playback device comprising a receiver configured to receive an audio signal, a processor for spectrally modifying the audio signal in a substantially real time manner to compensate for an auditory system disorder, and an output for outputting the spectrally modified audio signal to a person.

28. A method for providing relief to a subject experiencing tinnitus or conditions of reduced tolerance of loud sounds, the method comprising administering to said subject (a) an effective amount of at least one pharmaceutical agent and (b) at least one auditory system disorder device.

29. A method for using at least one pharmaceutical agent in the manufacture of a kit for use in treating an auditory system disorder, wherein the kit comprises said at least one pharmaceutical agent and at least one auditory system disorder device.

30. The method of claim 29, wherein the pharmaceutical agent comprises one or more anxiolytic agents, antidepressant agents, anticonvulsants, antiarrthymic agents, antihistamines, muscarinic agents. Hi antagonists, opioid agents, osmotic regulators, vasodilators, anesthetic agents. NMDA receptor antagonists, diuretics, or combinations or mixtures thereof.
